(19) United States
(12) Patent Application Publication
(10) Pub. No.: US 2025/0019745 A1
(43) Pub. Date: Jan. 16, 2025

(54) METHODS OF GENERATING SELF-REPLICATING RNA MOLECULES

(71) Applicant: Replicate Bioscience, Inc., San Diego, CA (US)

(72) Inventors: Nathaniel Stephen WANG, San Diego, CA (US); Shigeki Joseph MIYAKE-STONER, San Diego, CA (US); Parinaz ALIAHMAD, San Diego, CA (US)

(21) Appl. No.: 18/714,038

(22) PCT Filed: Nov. 28, 2022
(86) PCT No.: PCT/US2022/080515

§ 371 Date: May 28, 2024

Related U.S. Application Data
(60) Provisional application No. 63/283,660, filed on Nov. 29, 2021.

(51) Int. Cl. C12Q 1/6809

(52) U.S. Cl. CPC C12Q 1/6809

(57) ABSTRACT

The present disclosure generally relates to new processes for generating self-replicating RNA (srRNA) systems with superior expression properties. The disclosure also provides nucleic acids and recombinant cells expressing such srRNA constructs as well as pharmaceutical compositions containing the same. Further provided are compositions and methods for inducing pharmacodynamic effects in a subject and for the prevention and/or treatment of various health conditions.

### CROSS-REFERENCE TO RELATED APPLICATIONS

This application claims the benefit of priority to U.S. Provisional Patent Application Ser. No. 63/283,660, filed on Nov. 29, 2021. The disclosure of the above-referenced application is herein expressly incorporated by reference it its entirety, including any drawings.

### FIELD

The present disclosure relates to the field of immunology and medicine, and particularly relates to new processes for generating replicon, e.g., self-replicating RNA (srRNA) molecules, with superior expression properties. The disclosure also provides nucleic acids and recombinant cells expressing such srRNA molecules as well as pharmaceutical compositions containing the same. Further provided are compositions and methods for inducing pharmacodynamic effects in a subject and for the prevention and/or treatment of various health conditions.

### INCORPORATION OF THE SEQUENCE LISTING

The material in the accompanying Sequence Listing is hereby incorporated by reference into this application. The accompanying Sequence Listing text file, named 058462-506001WO_Sequence Listing_ST26.xml, was created on Nov. 22, 2022, and is 14 KB.

### BACKGROUND

In recent years, several different groups of animal viruses have been subjected to genetic manipulation either by homologous recombination or by direct engineering of their genomes. The availability of reverse genetics systems for both DNA and RNA viruses has created new perspectives for the use of recombinant viruses, for example, as vaccines, expression vectors, anti-tumor agents, in vivo gene therapy vectors, and drug delivery vehicles.

Recent advances in this regard include recombinant replicons, e.g., self-replicating RNA (srRNA), which are derived from the genomes of positive strand RNA viruses and represent a powerful tool for approaches to novel safe and effective vaccines and drug delivery vehicles. In particular, srRNA maintains auto-replicative activity derived from an RNA virus vector and therefore can replicate in host cells leading to an amplification of the amount of RNA encoding the desired gene product, and can be used to enhance efficiency of RNA delivery and/or expression of the encoded gene products for therapeutic and/or prophylactic applications. For example, in vaccine applications, srRNA-based vaccines are beneficial compared to non-replicative RNAs (e.g., mRNA) as it maintains the advantages of mRNA vaccines, such as rapid development, modular design, and cell-free synthesis, but requires a lower dose of RNA due to the self-replicative properties. This reduces the burden of manufacturing for both the drug substance and product and is potentially advantageous in the context of pandemic response as it would enable a greater percentage of the population to be vaccinated in a shorter amount of time.

While immense progress was made in srRNA technologies in recent years, there remains a need for additional methods for rapidly generating new srRNA-based expression tools for in vivo or ex vivo expression of gene products, such as therapeutic polypeptides, in quantities and for a period of time sufficient to produce therapeutic and/or prophylactic benefits.

### SUMMARY

The present disclosure relates generally to new methods for the identification and/or characterization of self-replicating RNA (srRNA) constructs, e.g., replicons, with superior expression properties that are suitable for expression of recombinant polypeptides of interest such as, e.g., antigen molecules and biotherapeutic molecules in cell cultures or in living bodies for prophylactic and/or therapeutic applications. Also disclosed are nucleic acids and recombinant cells expressing such srRNA constructs as well as pharmaceutical compositions containing the same. Also disclosed herein are methods for inducing a pharmacodynamic effect in a subject and, in particular, methods for eliciting an immune response and methods for preventing and/or treating a health condition in a subject in need thereof, wherein the methods include prophylactically or therapeutically administering one or more of the srRNA constructs, nucleic acid constructs, recombinant cells, and/or the pharmaceutical composition of the disclosure.

In one aspect of the disclosure, provided herein are methods for identifying and/or characterizing a self-replicating (srRNA) RNA construct, e.g., replicon, the method include: (a) providing a plurality of srRNA expression constructs each including a coding sequence for a polypeptide construct of interest (PCI) operably inserted into an alphavirus srRNA vector, wherein at least a portion of the coding sequence for the alphavirus structural proteins has been replaced with the coding sequence of the PCI; (b) analyzing level and/or functionality of the PCIs that are expressed from the plurality of srRNA expression constructs to identify one or more candidate PCIs having a defined property; (c) incorporating the srRNA expression constructs capable of expressing the candidate PCIs identified in (b) with at least one delivery vehicle to create a combinatorial collection of delivery systems; and (d) analyzing the delivery systems for their capability to confer at least one pharmacodynamic effect in a subject to identify a srRNA expression construct capable of conferring a desired pharmacodynamic effect.

Non-limiting exemplary embodiments of the methods of the disclosure can include one or more of the following features. In some embodiments, the coding sequence for the PCI includes a coding sequence for a single polypeptide (e.g., monogenic PCI) or coding sequences for a plurality of polypeptides (e.g., multigenic PCI). In some embodiments, the coding sequences of the plurality of polypeptides are operably linked to one another within a single open reading frame (i.e., in a polycistronic ORF). In some embodiments, the plurality of polypeptides are operably linked to one another by one or more connector sequences. In some embodiments, a connector sequence of the plurality of connector sequences includes an autoproteolytic peptide sequence. In some embodiments, the autoproteolytic peptide sequence includes one or more autoproteolytic cleavage sequences derived from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

In some embodiments, the coding sequences of the plurality of polypeptides are operably linked to one another by one or more an internal ribosomal entry sites (IRES). In some embodiments, the one or more IRES is selected from a viral IRES, a cellular IRES, and an artificial IRES. In some embodiments, the one or more IRES is selected from a Kaposi's sarcoma-associated herpesvirus (KSHV) IRES, a hepatitis virus IRES, a Pestivirus IRES, a Cripavirus IRES, a Rhopalosiphum padi virus IRES, a fibroblast growth factor IRES, a platelet-derived growth factor IRES, a vascular endothelial growth factor IRES, an insulin-like growth factor IRES, a picornavirus IRES, an encephalomyocarditis virus (EMCV) IRES, a Pim-1 IRES, a p53 IRES, an Apaf-1 IRES, a TDP2 IRES, an L-myc IRES, and a c-myc IRES.

In some embodiments, the PCI includes one or more polypeptides selected from microbial proteins, viral proteins, bacterial proteins, fungal proteins, mammalian proteins, and combinations of any thereof. In some embodiments, the PCI includes one or more polypeptides selected from antigen molecules, biotherapeutic molecules, or combinations of any thereof. In some embodiments, the PCI includes one or more antigen polypeptides selected from tumor-associated antigens, tumor-specific antigens, neoantigens, and combinations of any thereof. In some embodiments, the one or more antigen polypeptides includes estrogen receptors, intracellular signal transducer enzymes, and human epidermal growth receptors. In some embodiments, the one or more antigen polypeptides is selected from ESR1, PI3K, HER2, HER3, variants of any thereof, and combinations of any thereof. In some embodiments, the PCI includes one or more biotherapeutic polypeptides selected from immunomodulators, modulators of angiogenesis, modulators of extracellular matrix, modulators of metabolism, neurological modulators, and combinations of any thereof. In some embodiments, the PCI includes one or more cytokines selected from chemokines, interferons, interleukins, lymphokines, and tumor necrosis factors. In some embodiments, the PCI includes one or more interleukins selected from IL-1α, IL-1β, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ and subunits of any thereof. In some embodiments, the one or more biotherapeutic polypeptides is selected from IL-12A, IL-12B, IL-1RA, and combinations of any thereof.

In some embodiments, step (a) of the methods described herein includes providing a plurality of srRNA expression constructs each including a coding sequence for a variant of the PCI. In some embodiments, the providing in (a) includes: (i) obtaining an alphavirus srRNA expression vector, wherein at least a portion of encoding sequence for the alphavirus structural proteins has been replaced with a coding sequence for a polypeptide construct of interest (PCI); and (ii) generating a plurality of srRNA expression constructs each including a coding sequence for a variant of the PCI. In some embodiments, the providing in (a) includes: (i) obtaining coding sequences for a plurality of variants of a polypeptide construct of interest (PCI); and (ii) generating a plurality of srRNA expression constructs each including a coding sequence for a PCI variant of the plurality of PCI variants from (a) operably inserted into an alphavirus srRNA vector, wherein at least a portion of the encoding sequence for the alphavirus structural proteins has been replaced with the coding sequence of the PCI variant.

In some embodiments of the methods described herein, a PCI variant of the plurality of PCI variants includes one or more molecular alterations. In some embodiments, the one or more molecular alterations in the PCI variant is selected from the group consisting of deletions, substitutions, insertion, duplications, mutations, frameshift variants, splice variants, and combinations of any thereof. In some embodiments, the one or more molecular alterations are configured into a plurality of alteration cassettes arranged in tandem along the length of the antigen sequence. In some embodiments, an alteration cassette of the plurality of alteration cassettes includes one, two, three, four, five, or more molecular alterations. In some embodiments, the plurality of alteration cassettes are operably linked to one another by one or more linkers. In some embodiments, a linker of the one or more linkers includes a synthetic compound linker or a peptide linker. In some embodiments, the peptide linker includes an amino acid sequence selected from the group consisting of AAY, EAAAK (SEQ ID NO: 1), RVRR (SEQ ID NO: 2), GGGGS (SEQ ID NO: 3), and GPGPG (SEQ ID NO: 4).

In some embodiments, the analyzing level and/or functionality of the PCIs in step (b) is carried out in vitro, in vivo, or ex vivo. In some embodiments, the analyzing level and/or functionality of the PCIs includes immunoblotting analysis, fluorescence flow cytometry analysis, enzyme-linked immunoassay analysis, immunogenicity analysis, bioactivity analysis, and/or efficacy in a disease model. In some embodiments, the analysis of the delivery systems for their capacity to confer at least one pharmacodynamic effects in step (d) is carried out in vivo or ex vivo. In some embodiments, the at least one pharmacodynamic effect includes one or more of the following: immunogenicity effect, a biomarker response, a therapeutic effect, a prophylactic effect, a desired effect, an undesired effect, an adverse effect, and effect in a disease model. In some embodiments, the at least one pharmacodynamic effect includes induction of an immune response.

In some embodiments of the methods described herein, the delivery systems include a physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof. In some embodiments, the LNP delivery system includes a cationic lipid, an ionizable cationic lipid, an anionic lipid, or a neutral lipid. In some embodiments, the LNP delivery system includes an ionizable cationic lipid selected from the group consisting of ALC-0315, C12-200, LN16, MC3, MD1, SM-102, and a combination of any thereof. In some embodiments, the LNP includes a cationic lipid selected from the group consisting of 98N12-5, C12-200, C14-PEG2000, DLin-KC2-DMA (KC2), DLin-MC3-DMA (MC3), XTC, MD1, 7C1, and a combination of any thereof. In some embodiments, the LNP includes a neutral lipid selected from the group consisting of DPSC, DPPC, POPC, DOPE, SM, and a combination of any thereof. In some embodiments, the LNP includes lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG).

In some embodiments, the lipids can be combined in any number of molar ratios to produce a LNP. In addition, the polynucleotide(s) can be combined with lipid(s) in a wide range of molar ratios to produce a LNP. In some embodiments where the delivery systems described herein include an LNP, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1.

In some embodiments, the lipid-based nanoparticles (LNPs) have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In some embodiments of the methods described herein, the recombinant alphavirus srRNA is of a virus belonging to the Alphavirus genus of the Togaviridae family. In some embodiments, the recombinant alphavirus srRNA is of an alphavirus belonging to the VEEV/EEEV group, or the SFV group, or the SINV group. In some embodiments, the alphavirus is Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu virus (NDUV), Madariaga virus (MADV), or Buggy Creek virus. In some embodiments, the alphavirus is VEEV, EEEV, CHIKV, or SINV. In some embodiments, the alphavirus is VEEV. In some embodiments, the alphavirus is EEEV. In some embodiments, the alphavirus is CHIKV. In some embodiments, the alphavirus is SINV.

In some embodiments of the methods described herein, the srRNA expression vector is identified as having an immune-inducing activity suitable for a prophylactic use and/or a therapeutic use. In some embodiments, the srRNA expression vector is identified as having an immune-inducing activity suitable for a prophylactic use. In some embodiments, the srRNA expression vector is identified as having an immune-inducing activity suitable for a therapeutic use.

In one aspect, provided herein are srRNA constructs identified according to a method described herein.

In another aspect, provided herein are nucleic acids encoding a srRNA construct described herein.

In yet another aspect, provided herein are recombinant cells including (a) a RNA construct as described herein; and/or (b) a nucleic acid as described herein. Non-limiting exemplary embodiments of the methods of the disclosure can include one or more of the following features. In some embodiments, the recombinant cell is a eukaryotic cell. In some embodiments, the recombinant cell is an animal cell. In some embodiments, the animal cell is a vertebrate animal cell or an invertebrate animal cell. In some embodiments, the animal cell is a mammalian cell. In some embodiments, the animal cell is an insect cell. In some embodiments, the insect cell is a mosquito cell. In some embodiments, the animal cell is an immune cell. In some embodiments, the immune cell is a B cell, a monocyte, a natural killer (NK) cell, a natural killer T (NKT) cell, a basophil, an eosinophil, a neutrophil, a dendritic cell (DC), a macrophage, a regulatory T cell, a helper T cell (T_{H}), a cytotoxic T cell (T_{CTL}), a memory T cell, a gamma delta (γδ) T cell, a hematopoietic stem cell, or a hematopoietic stem cell progenitor. In some embodiments, the immune cell is a B cell, a T cell, or a dendritic cell (DC).

In a related aspect, provided herein are cell cultures including at least one recombinant cell as disclosed herein, and a cell culture medium.

In yet another aspect, provided herein are compositions, e.g., pharmaceutical compositions, including a therapeutically acceptable excipient and one or more of the following: (a) a RNA construct as described herein; and/or (b) a nucleic acid as described herein; and (c) a recombinant cell as described herein.

Non-limiting exemplary embodiments of the compositions as described herein can include one or more of the following features. In some embodiments, the composition is formulated as a vaccine. In some embodiments, the vaccine is a therapeutic vaccine. In some embodiments, the composition is formulated as an immunogenic composition. In some embodiments, the composition is formulated as a biotherapeutic. In some embodiments, the composition is formulated with a delivery vehicle into a delivery system. In some embodiments, the delivery system includes a physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof. In some embodiments, the LNP delivery system includes a cationic lipid, an ionizable cationic lipid, an anionic lipid, or a neutral lipid. In some embodiments, the LNP includes an ionizable cationic lipid selected from the group consisting of ALC-0315, C12-200, LN16, MC3, MD1, SM-102, and a combination of any thereof. In some embodiments, the LNP includes a cationic lipid selected from the group consisting of 98N12-5, C12-200, C14-PEG2000, DLin-KC2-DMA (KC2), DLin-MC3-DMA (MC3), XTC, MD1, 7C1, and a combination of any thereof. In some embodiments, the LNP includes a neutral lipid selected from the group consisting of DPSC, DPPC, POPC, DOPE, SM, and a combination of any thereof. In some embodiments, the LNP includes a lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, GL67A-DOPE-DMPE-polyethylene glycol (PEG), and a combination of any thereof. In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles (LNPs) have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In another aspect, provided herein are methods for inducing a pharmacodynamic effect in a subject, the methods include administering to the subject a composition including one or more of the following: (a) a srRNA construct as described herein; (b) a nucleic acid as described herein; (c) a recombinant cell as described herein; and (d) a pharmaceutical composition as described herein. In some embodiments, the pharmacodynamic effect includes eliciting an immune response in the subject. In another aspect, provided herein are methods for preventing or treating a health condition in a subject, the methods include prophylactically or therapeutically administering to the subject a composition including one or more of the following: (a) a srRNA construct as described herein; (b) a nucleic acid as described herein; (c) a recombinant cell as described herein; and (d) a pharmaceutical composition as described herein.

Non-limiting exemplary embodiments of the methods for inducing a pharmacodynamic effect, and/or preventing, and/or treating a health condition in a subject as described herein can include one or more of the following features. In some embodiments, the administered composition induces production of one or more pro-inflammatory molecules in the subject. In some embodiments, the one or more pro-inflammatory molecules includes interferon gamma (IFNγ), cytokines, TNF-α, GM-CSF, and MIP1α, granzyme B, granzyme A, perforin, or a combination of any thereof. In some embodiments, the subject has been previously treated with one or more therapies and has developed at least a partial resistance to said one or more therapies. In some embodiments, at least one of the one or more therapies includes a small molecule. In some embodiments, the health condition is a proliferative disorder, inflammatory disorder, autoimmune disorder, or a microbial infection. In some embodiments, the subject has or is suspected of having a health condition associated with proliferative disorder, inflammatory disorder, autoimmune disorder, or a microbial infection. In some embodiments, the proliferative disorder is a cancer. In some embodiments, the cancer is a breast cancer. In some embodiments, the composition is administered to the subject individually as a single therapy (monotherapy) or as a first therapy in combination with at least one additional therapies. In some embodiments, the at least one additional therapies is selected from the group consisting of chemotherapy, radiotherapy, immunotherapy, hormonal therapy, toxin therapy, targeted therapy, and surgery.

In another aspect, provided herein are kits for practicing a method of the disclosure, for example for eliciting an immune response, for the prevention, and/or treatment of a health condition, the kits include one or more of the following: (a) a srRNA construct as described herein; (b) a nucleic acid construct as described herein; (c) a recombinant cell as described herein; and (d) a pharmaceutical composition as described herein.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIG. 1 is a flow diagram illustrating a non-limiting example of a method for identifying and/or characterizing a self-replicating RNA (srRNA) construct in accordance with some embodiments of the disclosure.

FIG. 2 is graphical representation of immunogenicity in mice used to optimize design of a mutant ESR1 antigen cassette. The X-axis lists the ordinality of ESR1 mutations K303R, E380Q, Y537C, Y537S, Y537N, and D538G within the gene cassette and the use of linkers connecting these mutations. The Y-axis shows the total T cell responses using peptides encoding ESR1 sequences in an ELISpot assay.

FIG. 3 are figures showing protein expression from BHK-21 cells transfected monogenic, bigenic, or tetragenic srRNAs encoding ESR1, PI3K, HER2, and HER3 from a panel of constructs having various molecular configurations and comparing them to monogenic constructs. Protein expression levels of monogenic constructs are assigned a value of “1”, and relative expression of each gene in bigenics or tetragenic configurations are compared with monogenic construct protein expression levels. FIG. 3A is an immunoblot for ESR1 protein expression. FIG. 3B is a chart showing the relative ESR1 expression based on the signal intensity of the immunoblot bands. FIG. 3C is an immunoblot for PI3K protein expression. FIG. 3D is a chart showing the relative PI3K expression based on the signal intensity of the immunoblot bands. FIG. 3E is a chart showing the relative expression of HER2 from based on the mean fluorescence intensity (MFI) quantified by fluorescence flow cytometry (FFC) after straining with an Alexa Fluor® 488 (AF488) labeled, HER2-specific antibody. FIG. 2E is a chart showing the relative expression of HER3 from based on the MFI quantified by FFC after straining with an allophycocyanin (APC) labeled, HER3-specific antibody. FIG. 2G is a spider chart summarizing the protein expression readout of ESR1, PI3K, HER2, and HER3 in the panel of constructs.

FIG. 4 is a graphical representation of T cell responses in mice administered constructs having various molecular configurations. The x-axis shows different constructs, in either monogenic, bigenic, or tetragenic form, having different ordinalities of ESR1, PI3K, HER2, and HER3. The Y-axis shows the total T cell responses to peptides encoding sequences derived from ESR1 mutations, HER2, and HER3 in an ELISpot assay. PI3K responses were not measured in this experiment because it does not form responses in BALB/c mice.

FIG. 5 is a schematic of an exemplary neoantigen cassette. Peptides containing mutations are separated by linkers to create a single cassette.

FIG. 6 is a graphical representation of T cell responses in mice administered constructs having different srRNA vectors and formulated in two different lipid nanoparticles, differing in the cationic lipid either LNP1 (“L1”) or LNP2 (“L2”) in the composition.

FIG. 7 is a schematic describing two types (i.e., therapeutic and prophylactic) of estrogen receptor positive breast cancer efficacy studies to model human disease.

FIGS. 8A-8B are graphical representations of in vitro protein expression from monogenic and multigenic VEEV srRNA constructs. Supernatants of transfected BHK-21 cells with each srRNA construct were used to measure protein expression determined by ELISA. FIG. 8A shows the results from an IL-12 ELISA. The x-axis shows different constructs tested. FIG. 8B shows the results from an TL-RA ELISA. The x-axis shows different constructs tested.

FIGS. 9A-9B are graphical representations of in vitro protein bioactivity from monogenic and multigenic constructs in VEE. Supernatants of transfected BHK-21 cells with each srRNA construct were used to measure protein bioactivity of cytokines on reporter cells expressing their cognate receptors. FIG. 9A shows results from an IL-12 bioassay. The x-axis shows different constructs tested. FIG. 9B shows results from an IL-1RA bioactivity assay. The x-axis shows different constructs tested.

FIG. 10 is a graphical representation of combined IL-12 bioactivity and IL-1RA expression data from srRNA constructs.

FIGS. 11A-11B are graphical representations of protein expression from the two best multigenic configurations cloned into six different srRNA vectors. FIG. 11A shows the results from an IL-12 ELISA. The x-axis shows different constructs tested. FIG. 11B shows the results from an TL-RA ELISA. The x-axis shows different constructs tested.

FIGS. 12A-12B are graphical representations of protein bioactivity from the two best multigenic configurations cloned into six different srRNA vectors. FIG. 12A shows the results from an IL-12 bioassay. The x-axis shows different constructs tested. FIG. 12B shows the results from an TL-RA bioassay. The x-axis shows different constructs tested.

FIG. 13 is a graphical representation summarizing the L-12 and IL-1RA bioactivity of the two best multigenic configurations in six different srRNA vectors.

FIGS. 14A-14B are graphical representations of in vivo protein expression in mouse serum by the two best bigenic configurations in six different srRNA vectors. FIG. 14A shows the results from an IL-12 ELISA. The x-axis shows different constructs tested. FIG. 14B shows the results from an IL-1RA ELISA. The x-axis shows different constructs tested.

FIGS. 15A-15B are graphical representations of in vivo protein expression from different formulations of optimal srRNA constructs. FIG. 15A shows the results from an IL-12 ELISA. The x-axis shows different constructs and formulations tested. FIG. 15B shows the results from an TL-RA ELISA. The x-axis shows different constructs and formulations tested.

FIGS. 16A-16B are bar charts illustrating in vivo immunogenicity of a panel of srRNAs encoding an exemplary viral antigen, which is an envelope glycoprotein G of a rabies virus (RABV-G). The panel included srRNAs derived from Venezuelan equine encephalitis virus (VEE.TC83), Chikungunya virus strains S27 (CHIK.S27) and DRDE-06 (CHIK.DRDE), Sindbis virus strains Girdwood (SIN.GW) and AR86-Girdwood Hybrid 1 (SIN.AR86), and Eastern equine encephalitis virus (EEE.FL93). FIG. 16A shows the quantification of antigen-specific splenic T cell responses evaluated by ELISpot after two immunizations. FIG. 16B shows anti-rabies neutralizing antibody titers from sera after two immunizations.

### DETAILED DESCRIPTION OF THE DISCLOSURE

The present disclosure relates generally to new methods for the identification and/or characterization of self-replicating RNA (srRNA) constructs, e.g., replicons, with superior expression properties that are suitable for expression of recombinant polypeptides of interest such as, e.g., antigen molecules and biotherapeutic molecules in cell cultures or in living bodies for prophylactic and/or therapeutic applications. Accordingly, the srRNA constructs identified by the methods disclosed herein are also within the scope of the disclosure. Some aspects and embodiments of the disclosure relate to recombinant nucleic acids and recombinant cells that have been engineered to express the srRNA constructs disclosed herein as well as pharmaceutical compositions containing the same. Some other aspects and embodiments of the disclosure relate to compositions and methods for inducing pharmacodynamic effects in a subject and for the prevention and/or treatment of various health conditions.

As described above, the availability of reverse genetics systems for both DNA and RNA viruses in recent years has created new perspectives for the use of recombinant viruses, for example, as vaccines, expression vectors, anti-tumor agents, gene therapy vectors, and drug delivery vehicles. For example, the application of modified viral vectors for gene expression in host cells continues to expand. In particular, many viral-based expression vectors have been deployed for expression of heterologous proteins in cultured recombinant cells. Recent advances in this regard include further development of techniques and systems for production of multi-subunit protein complexes, and co-expression of protein-modifying enzymes to improve heterologous protein production. Other recent progresses regarding viral expression vector technologies include many advanced genome engineering applications for controlling gene expression, preparation of viral vectors, in vivo gene therapy applications, and creation of vaccine delivery vectors.

Self-amplifying RNA (srRNA) constructs as described herein, e.g., when delivered into a cell (e.g., host cell) or a subject, can amplify themselves and initiate expression and/or overexpression of heterologous gene products in the host cell or subject. Self-replicating RNA constructs (e.g., replicons) of the disclosure, unlike mRNA, use their own encoded viral polymerase to amplify itself. Particular srRNA constructs of the disclosure, such as those based on alphaviruses, generate large amounts of subgenomic mRNAs from which large amounts of heterologous proteins can be expressed.

In some embodiments, srRNA as described herein are based on RNA viruses (e.g., alphaviruses) and can be used as robust expression systems. For example, it has been reported that an advantage of using alphaviruses such as Chikungunya virus (CHIKV), Eastern Equine Encephalitis virus (EEEV), Sindbis virus (SINV) as viral expression vectors is that they can direct the synthesis of large amounts of recombinant proteins in recombinant host cells. Among other advantages, polypeptides such as therapeutic single chain antibodies can be most effective if expressed at high levels in vivo. In addition, for producing recombinant antibodies purified from cells in culture (ex vivo), high protein expression from a srRNA (e.g., replicon) can increase overall yields of the antibody product. Furthermore, if the protein being expressed is a vaccine antigen, high level expression can induce the most robust immune response in vivo.

In the following detailed description, reference is made to the accompanying drawings, which form a part hereof. In the drawings, similar symbols generally identify similar components, unless context dictates otherwise. The illustrative alternatives described in the detailed description, drawings, and claims are not meant to be limiting. Other alternatives may be used and other changes may be made without departing from the spirit or scope of the subject matter presented here. It will be readily understood that the aspects, as generally described herein, and illustrated in the Figures, can be arranged, substituted, combined, and designed in a wide variety of different configurations, all of which are explicitly contemplated and make part of this application.

### Definitions

Unless otherwise defined, all terms of art, notations, and other scientific terms or terminology used herein are intended to have the meanings commonly understood by those of skill in the art to which this application pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

The terms “administration” and any grammatical variation thereof, as used herein, refer to the delivery of a bioactive composition or formulation by an administration route comprising, but not limited to, intranasal, transdermal, intravenous, intra-arterial, intramuscular, intranodal, intraperitoneal, subcutaneous, intramuscular, oral, intravaginal, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications can occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “construct” refers to a recombinant molecule, e.g., recombinant nucleic acid or polypeptide, including one or more nucleic acid sequences or amino acid sequences from heterologous sources. For example, polypeptide constructs can be chimeric polypeptide molecules in which two or more amino acid sequences of different origin are operably linked to one another in a single polypeptide construct. Similarly, nucleic acid constructs can be chimeric nucleic acid molecules in which two or more nucleic acid sequences of different origin are assembled into a single nucleic acid molecule. Representative nucleic acid constructs can include any recombinant nucleic acid molecules, linear or circular, single stranded or double stranded DNA or RNA nucleic acid molecules, derived from any source, such as a plasmid, cosmid, virus, autonomously replicating polynucleotide molecule, phage, capable of genomic integration or autonomous replication, comprising a nucleic acid molecule where one or more nucleic acid sequences have been operably linked. In some embodiments, one or more nucleic acid constructs can be incorporated (e.g., inserted) within a single nucleic acid molecule, such as a single vector, or can be incorporated (e.g., inserted) within two or more separate nucleic acid molecules, such as two or more separate vectors. The term “vector” is used herein to refer to a nucleic acid molecule or sequence capable of transferring or transporting another nucleic acid molecule. Thus, the term “vector” encompasses both DNA-based vectors and RNA-based vectors. The term “vector” includes cloning vectors and expression vectors, as well as viral vectors and integrating vectors. An “expression vector” is a vector that includes a regulatory region, thereby capable of expressing DNA sequences and fragments in vitro, ex vivo, and/or in vivo. In some embodiments, a vector may include sequences that direct autonomous replication in a cell such as, for example a plasmid (DNA-based vector) or a self-replicating RNA vector. In some embodiments, a vector may include sequences sufficient to allow integration into host cell DNA. In some embodiments, a vector may include DNA sequences that can be transcribed into RNA in vitro and/or in vivo. Useful vectors include, for example, plasmids (e.g., DNA plasmids or RNA plasmids), transposons, cosmids, bacterial artificial chromosomes, and viral vectors. In some embodiments, the vector of the disclosure can be single-stranded vector (e.g., ssDNA or ssRNA). In some embodiments, the vector of the disclosure can be double-stranded vector (e.g., dsDNA or dsRNA). In some embodiments, a vector is a gene delivery vector. In some embodiments, a vector is used as a gene delivery vehicle to transfer a gene into a cell. In some embodiments, the vector of the disclosure is a self-replicating RNA (srRNA) vector.

The term “effective amount”, “therapeutically effective amount”, or “pharmaceutically effective amount” of a composition of the disclosure, e.g., srRNA constructs, nucleic acid constructs, recombinant cells, and/or pharmaceutical compositions, generally refers to an amount sufficient for the composition to accomplish a stated purpose relative to the absence of the composition (e.g., achieve the effect for which it is administered, stimulate an immune response, prevent or treat a disease, or reduce one or more symptoms of a disease, disorder, infection, or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

The term “naked” as used herein when referencing nucleic acids that are substantially free of other macromolecules, such as lipids, polymers, and proteins. A “naked” nucleic acid, such as a self-replicating RNA (e.g., replicon), is not formulated with other macromolecules to improve cellular uptake. Accordingly, a naked nucleic acid is not encapsulated in, absorbed on, or bound to a liposome, a microparticle, a nanoparticle, a cationic emulsion, and the like.

The term “operably linked”, as used herein, denotes a physical or functional linkage between two or more elements, e.g., polypeptide sequences or polynucleotide sequences, which permits them to operate in their intended fashion. For example, the term “operably linked” when used in context of the nucleic acid molecules and srRNA constructs described herein or the coding sequences and promoter sequences in a nucleic acid construct means that the coding sequences and promoter sequences are in-frame and in proper spatial and distance away to permit the effects of the respective binding by transcription factors or RNA polymerase on transcription. It should be understood that operably linked elements may be contiguous or non-contiguous (e.g., linked to one another through a linker).

In the context of polypeptide constructs, “operably linked” refers to a physical linkage (e.g., directly or indirectly linked) between amino acid sequences (e.g., different segments, portions, or domains) to provide for a described activity of the constructs. In the present disclosure, region, or domains of the constructs of the disclosure may be operably linked to retain proper folding, processing, targeting, expression, binding, and other functional properties of the constructs in the cell. Unless stated otherwise, the segments, portions, and domains of the constructs of the disclosure are operably linked to each other. Operably linked segments, portions, and domains of the constructs disclosed herein may be contiguous or non-contiguous (e.g., linked to one another through a linker).

The term “portion” as used herein refers to a fraction. With respect to a particular structure such as a polynucleotide sequence or an amino acid sequence or a polypeptide, the term “portion” thereof may designate a continuous or a discontinuous fraction of said structure. For example, a portion of an amino acid sequence comprises at least 1%, at least 5%, at least 10%, at least 20%, at least 30%, at least 40%, at least 50%, at least 60%, at least 70%, at least 80%, and at least 90% of the amino acids of said amino acid sequence. In addition or alternatively, if the portion is a discontinuous fraction, said discontinuous fraction is composed of 2, 3, 4, 5, 6, 7, 8, or more parts of a structure (e.g., domains of a protein), each part being a continuous element of the structure. For example, a discontinuous fraction of an amino acid sequence may be composed of 2, 3, 4, 5, 6, 7, 8, or more, for example not more than 4 parts of said amino acid sequence, wherein each part comprises at least 1, at least 2, at least 3, at least 4, at least 5 continuous amino acids, at least 10 continuous amino acids, at least 20 continuous amino acids, or at least 30 continuous amino acids of the amino acid sequence.

The term “pharmaceutically acceptable excipient” as used herein refers to any suitable substance that provides a pharmaceutically acceptable carrier, additive, or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” when used with reference to a cell, a nucleic acid, a protein, or a vector, indicates that the cell, nucleic acid, protein or vector has been altered or produced through human intervention such as, for example, has been modified by or is the result of laboratory methods. Thus, for example, recombinant proteins and nucleic acids include proteins and nucleic acids produced by laboratory methods. Recombinant proteins can include amino acid residues not found within the native (non-recombinant or wild-type) form of the protein or can be include amino acid residues that have been modified, e.g., labeled. The term can include any modifications to the peptide, protein, or nucleic acid sequence. Such modifications may include the following: any chemical modifications of the peptide, protein or nucleic acid sequence, including of one or more amino acids, deoxyribonucleotides, or ribonucleotides; addition, deletion, and/or substitution of one or more of amino acids in the peptide or protein; creation of a fusion protein, e.g., a fusion protein comprising an antibody fragment; and addition, deletion, and/or substitution of one or more of nucleic acids in the nucleic acid sequence. The term “recombinant” when used in reference to a cell is not intended to include naturally-occurring cells but encompass cells that have been engineered/modified to include or express a polypeptide or nucleic acid that would not be present in the cell if it was not engineered/modified.

As used herein, a “subject” or an “individual” includes animals, such as human (e.g., human individuals) and non-human animals. In some embodiments, a “subject” or “individual” is a patient under the care of a physician. Thus, the subject can be a human patient or an individual who has, is at risk of having, or is suspected of having a health condition of interest (e.g., cancer or infection) and/or one or more symptoms of the health condition. The subject can also be an individual who is diagnosed with a risk of the health condition and/or disease of interest at the time of diagnosis or later. The term “non-human animals” includes all vertebrates, e.g., mammals, e.g., rodents, e.g., mice, livestock, domesticated animals and pets, non-human primates, and other mammals, such as e.g., sheep, cats, dogs, cows, chickens, and non-mammals, such as amphibians, reptiles, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number. If the degree of approximation is not otherwise clear from the context, “about” means either within plus or minus 10% of the provided value, or rounded to the nearest significant figure, in all cases inclusive of the provided value. In some embodiments, the term “about” indicates the designated value±up to 10%, up to ±5%, or up to ±1%.

Headings, e.g., (a), (b), (i) etc., are presented merely for ease of reading the specification and claims. The use of headings in the specification or claims does not require the steps or elements be performed in alphabetical or numerical order or the order in which they are presented.

It is understood that aspects and embodiments of the disclosure described herein include “comprising”, “consisting”, and “consisting essentially of” aspects and embodiments. As used herein, “comprising” is synonymous with “including”, “containing”, or “characterized by”, and is inclusive or open-ended and does not exclude additional, unrecited elements or method steps. As used herein, “consisting of” excludes any elements, steps, or ingredients not specified in the claimed composition or method. As used herein, “consisting essentially of” does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes or gene products disclosed herein, which in some embodiments relate to mammalian nucleic acid and amino acid sequences, are intended to encompass homologous and/or orthologous genes and gene products from other animals including, but not limited to other mammals, fish, amphibians, reptiles, and birds. In some embodiments, the genes, nucleic acid sequences, amino acid sequences, peptides, polypeptides and proteins are human. The term “gene” is also intended to include variants thereof.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Alphaviruses

Alphaviruses are small, enveloped RNA viruses with a single-stranded, positive-sense RNA genome. The alphavirus genus includes, inter alia, the Sindbis virus (SINV), the Semliki Forest virus (SFV), the Ross River virus (RRV), Venezuelan equine encephalitis virus (VEEV), and Eastern equine encephalitis virus (EEEV), which are all closely related and are able to infect various vertebrates such as mammalians, rodents, fish, avian species, and larger mammals such as humans and horses as well as invertebrates such as insects. In particular, the Sindbis and the Semliki Forest viruses have been widely studied and the life cycle, mode of replication, etc., of these viruses are well characterized. Non-limiting exemplary alphavirus species suitable for the compositions and methods disclosed herein include Aura virus (AURAV), Babanki virus (BABV), Barmah Forest virus (BFV), Bebaru virus (BEBV), Buggy Creek virus, Caaingua virus, Cabassou virus, Chikungunya virus (CHIKV), Eastern equine encephalitis virus (EEEV), Eilat virus, Everglades virus (EVEV), Fort Morgan virus (FMV), Getah virus (GETV), Highlands J virus (HJV), Kyzylagach virus (KYZV), Madariaga virus (MADV), Mayaro virus (MAYV), Middelburg virus (MIDV), Mosso das Pedras virus, Mucambo virus (MUCV), Ndumu virus (NDUV), O'nyong'nyong virus (ONNV), Pixuna virus (PIXV), Rio Negro virus (RNV), Ross River virus (RRV), Salmon pancreas disease virus (SPDV), Semliki Forest virus (SFV), Sindbis virus (SINV), Sleeping disease virus (SDV), Southern elephant seal virus (SESV), Tai Forest virus (TFV), Tonate virus, Trocara virus, Una virus (UNAV), Venezuelan equine encephalitis virus (VEEV), Western equine encephalitis virus (WEEV), and Whataroa virus (WHAV).

The alphavirus genome is approximately 12 kb long, and it consists of two open reading frames (ORFs): a 7 kb frame encoding the nonstructural proteins (nsPs) and a 4 kb frame encoding the structural polyprotein. The non-structural polyprotein (nsP) is cleaved into four different proteins (nsP1, nsP2, nsP3, and nsP4) which are necessary for the transcription and translation of viral mRNA inside the cytoplasm of host cells.

The nsP1 protein is an mRNA capping enzyme that possesses both guanine-7-methyltransferase (MTase) and guanylyltransferase (GTase) activities, where they direct the methylation and capping of newly synthesized viral genomic and subgenomic RNAs. The MTase motif in the N-terminal domain of nsP1 catalyzes the transfer of the methyl group from S-adenosylmethionine (AdoMet) to the N7 position of a GTP molecule (m7Gppp). GTase then binds the m7Gppp, forming a covalent link with a catalytic histidine (m7Gp-GTase) and releasing PPi. The GTase then transfers the m7Gp molecule to the 5′-diphosphate RNA to create m7GpppNp-RNA. The resulting cap structure is essential for viral mRNA translation and prevents the mRNA from being degraded by cellular 5′ exonucleases. Following the N-terminal domain are features that allow the association of the nsP1 protein to cellular membranes. The presence of α-helical amphipathic loop and palmitoylation sites allow the nsP1 protein and nsP1-containing replication complex to anchor onto the plasma membrane, possibly through nsP1 interaction with the membrane's anionic phospholipids.

The nsP2 protein possesses numerous enzymatic activities and functional roles. The N-terminal region contains a helicase domain that has seven signature motif of Superfamily 1 (SF1) helicases. It functions as an RNA triphosphatase that performs the first of the viral RNA capping reactions. It also functions as a nucleotide triphosphatase (NTPase), fueling the RNA helicase activity. The C-terminal region of nsP2 contains a papain-like cysteine protease, which is responsible for processing the viral non-structural polyprotein. The protease recognizes conserved motifs within the polyprotein. This proteolytic function is highly regulated and is modulated by other domains of nsP2. The alphavirus nsP2 protein has also been described as a virulence factor responsible for the transcriptional and translational shutoff in infected host cells and the inhibition of interferon (IFN) mediated antiviral responses contributing to the controlling of translational machinery by viral factors.

The precise role(s) of alphavirus nsP3 protein in the replication complex is less clear. The nsP3 protein has three recognized domains: the N-terminal macrodomain with phosphatase activity and nucleic acid binding ability, the alphavirus unique domain (AUD) and the C-terminal hypervariable domain. It has been demonstrated that the deletion of this domain in SFV nsP3 resulted in low viral pathogenicity, suggesting its importance in viral RNA transcription regulation.

The nsP4 polymerase is the most highly conserved protein in alphaviruses, with the most divergent being >50% identical in amino acid sequence when compared with other alphaviral nsP4s. The nsP4 contains the core RNA-dependent RNA polymerase (RdRp) domain at the C-terminal end, determined to be solely responsible for the RNA synthetic properties of the viral replication complex. The RdRp participates in replicating the genomic RNA via a negative strand RNA and transcribing the 26S subgenomic RNA. The N-terminal domain is alphavirus-specific and can be partially disordered structurally.

The 5′ two-thirds of the alphavirus genome encodes a number of nonstructural proteins (nsPs) necessary for transcription and replication of viral RNA. These proteins are translated directly from the RNA and together with cellular proteins form the RNA-dependent RNA polymerase essential for viral genome replication and transcription of subgenomic RNA. Four nonstructural proteins (nsP1, nsP2, nsP3, nsP4) are produced as a single polyprotein constitute the virus' replication machinery. The processing of the polyprotein occurs in a highly regulated manner, with cleavage at the P2/3 junction influencing RNA template use during genome replication. This site is located at the base of a narrow cleft and is not readily accessible. Once cleaved, nsP3 creates a ring structure that encircles nsP2. These two proteins have an extensive interface. Mutations in nsP2 that produce noncytopathic viruses or a temperature sensitive phenotypes cluster at the P2/P3 interface region. P3 mutations opposite the location of the nsP2 noncytopathic mutations prevent efficient cleavage of P2/3. This in turn can affect RNA infectivity altering viral RNA production levels.

The 3′ one-third of the genome comprises subgenomic RNA which serves as a template for translation of all the structural proteins required for forming viral particles: the core nucleocapsid protein C, and the envelope proteins P62 and E1 that associate as a heterodimer. The viral membrane-anchored surface glycoproteins are responsible for receptor recognition and entry into target cells through membrane fusion. The subgenomic RNA is transcribed from the p26S subgenomic promoter present at the 3′ end of the RNA sequence encoding the nsP4 protein. The proteolytic maturation of P62 into E2 and E3 causes a change in the viral surface. Together the E1, E2, and sometimes E3, glycoprotein “spikes” form an E1/E2 dimer or an E1/E2/E3 trimer, where E2 extends from the center to the vertices, E1 fills the space between the vertices, and E3, if present, is at the distal end of the spike. Upon exposure of the virus to the acidity of the endosome, E1 dissociates from E2 to form an E1 homotrimer, which is necessary for the fusion step to drive the cellular and viral membranes together. The alphaviral glycoprotein E1 is a class II viral fusion protein, which is structurally different from the class I fusion proteins found in influenza virus and HIV. The E2 glycoprotein functions to interact with the nucleocapsid through its cytoplasmic domain, while its ectodomain is responsible for binding a cellular receptor. Most alphaviruses lose the peripheral protein E3, while in Semliki viruses it remains associated with the viral surface.

Alphavirus replication has been reported to take place on membranous surfaces within the host cell. In the first step of the infectious cycle, the 5′ end of the genomic RNA is translated into a polyprotein (nsP1-4) with RNA polymerase activity that produces a negative strand complementary to the genomic RNA. In a second step, the negative strand is used as a template for the production of two RNAs, respectively: (1) a positive genomic RNA corresponding to the genome of the secondary viruses producing, by translation, other nsP and acting as a genome for the virus; and (2) subgenomic RNA encoding the structural proteins of the virus forming the infectious particles. The positive genomic RNA/subgenomic RNA ratio is regulated by proteolytic autocleavage of the polyprotein to nsP1, nsP2, nsP3 and nsP4. In practice, the viral gene expression takes place in two phases. In a first phase, there is main synthesis of positive genomic strands and of negative strands. During the second phase, the synthesis of subgenomic RNA is virtually exclusive, thus resulting in the production of large amount of structural protein.

### Self-Replicating RNA

As will be appreciated by the skilled artisan, the term “self-replicating RNA” refers to RNA molecule that contains all of the genetic information required for directing its own amplification or self-replication within a permissive cell. Therefore, srRNA is sometimes also referred to as “self-amplifying RNA” (saRNA). In some embodiments, the srRNA is a “replicon,” which can be a linear or circular section of DNA or RNA which replicates sequentially as a unit. Non-limiting examples of replicons include “replicon RNA” or “RNA replicon.” To direct its own replication, the srRNA generally (1) encodes polymerase, replicase, or other proteins which may interact with viral or host cell-derived proteins, nucleic acids or ribonucleoproteins to catalyze the RNA amplification process; and (2) contain cis-acting RNA sequences required for replication and transcription of the subgenomic replicon-encoded RNA. These sequences may be bound during the process of replication to its self-encoded proteins, or non-self-encoded cell-derived proteins, nucleic acids or ribonucleoproteins, or complexes between any of these components. In some embodiments of the disclosure, the replicon, e.g., srRNA, is derived from an alphavirus. In some embodiments of the disclosure, an alphavirus srRNA construct (e.g., srRNA, saRNA, or replicon molecule) generally contains the following elements: 5′ viral or defective-interfering RNA sequence(s) required in cis for replication, sequences coding for biologically active alphavirus non-structural proteins (e.g., nsP1, nsP2, nsP3, and nsP4), a subgenomic promoter (sg) for the subgenomic RNA (sgRNA), 3′ viral sequences required in cis for replication, and optionally a polyadenylate tract (poly(A)). In some instances, a subgenomic promoter (sg) that directs expression of a heterologous sequence can be included in the srRNA construct of the disclosure.

Further, the term srRNA molecule (e.g., srRNA, saRNA, or replicon molecule) generally refers to a molecule of positive polarity, or “message” sense, and the srRNA may be of length different from that of any known, naturally-occurring alphavirus. In some embodiments of the present disclosure, the srRNA does not contain at least a portion of the coding sequence for one or more of the alphavirus structural proteins; and/or sequences encoding structural genes can be substituted with heterologous sequences. In those instances, where the srRNA is to be packaged into a recombinant alphavirus particle, it can contain one or more sequences, so-called packaging signals, which serve to initiate interactions with alphavirus structural proteins that lead to particle formation.

The srRNA constructs of the disclosure generally have a length of at least about 2 kb. For example, the srRNA can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the srRNA can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 Kb to about 10 kb, about 6 Kb to about 9 Kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the srRNA can have a length of about 6 kb to about 14 kb. In some embodiments, the srRNA can have a length of about 6 kb to about 16 kb.

Methods for Identification and/or Characterization of srRNA Constructs

As described in greater detail below, one aspect of the disclosure relates to methods for identifying and/or characterizing a srRNA construct, e.g., a srRNA construct having a desired expression property suitable for the expression of a recombinant polypeptide of interest such as, e.g., an antigen molecule and biotherapeutic molecule in a host cell, a cell culture, an ex-vivo cell-free expression system, or in a subject for prophylactic and/or therapeutic applications.

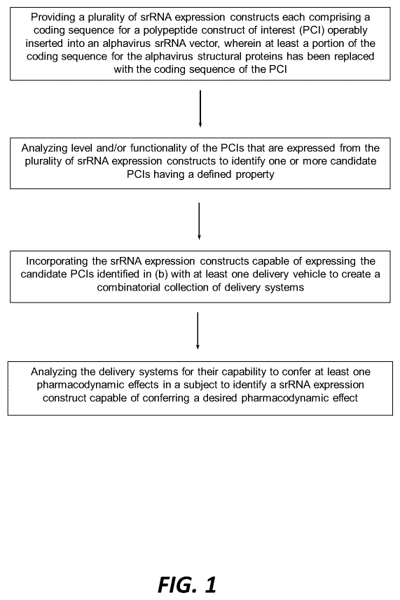

FIG. 1 is a flow diagram illustrating a non-limiting example of a method for identifying and/or characterizing a self-replicating RNA (srRNA, e.g., replicon) construct disclosed herein. As illustrated in FIG. 1, in some embodiments, a method of the disclosure can include the following operations: (a) providing a plurality of srRNA expression constructs each including a coding sequence for a polypeptide construct of interest (PCI) operably inserted into an alphavirus srRNA vector, wherein at least a portion of the coding sequence for the alphavirus structural proteins has been replaced with the coding sequence of the PCI; (b) analyzing level and/or functionality of the PCIs that are expressed from the plurality of srRNA expression constructs to identify one or more candidate PCIs having a defined property; (c) incorporating the srRNA expression constructs capable of expressing the candidate PCIs identified in (b) with at least one delivery vehicle to create a combinatorial collection of delivery systems; and (d) analyzing the delivery systems for their capability to confer at least one pharmacodynamic effect in a subject to identify a srRNA expression construct capable of conferring a desired pharmacodynamic effect.

Non-limiting exemplary embodiments of the methods of the disclosure can include one or more of the following features. In some embodiments, the alphavirus srRNA vector is devoid of at least a portion of the nucleic acid sequence encoding one or more of the viral structural proteins CP, E1, E2, E3, and 6K of the alphavirus srRNA vector. In some embodiments, the alphavirus srRNA vector is devoid of a portion of or the entire sequence encoding CP. In some embodiments, the alphavirus srRNA vector is devoid of a portion of or the entire sequence encoding E1. In some embodiments, the alphavirus srRNA vector is devoid of a portion of or the entire sequence encoding E2. In some embodiments, the alphavirus srRNA vector is devoid of a portion of or the entire sequence encoding E3. In some embodiments, the alphavirus srRNA vector is devoid of a portion of or the entire sequence encoding 6K. In some embodiments, the alphavirus srRNA vector is devoid of a portion of or the entire sequence encoding a combination of CP, E1, E2, E3, and 6K. In some embodiments of the disclosure, the coding sequence for nonstructural proteins nsP1, nsP2, nsP3, and nsP4 of the alphavirus srRNA vector is present, however at least a portion of or the entire sequence encoding one or more structural proteins (e.g., CP, E1, E2, E3, and 6K) of the alphavirus srRNA vector is absent.

In some embodiments, the alphavirus srRNA vector is devoid of a substantial portion of the nucleic acid sequence encoding one or more viral structural proteins. The skilled artisan will understand that a substantial portion of a nucleic acid sequence encoding a viral structural polypeptide can include enough of the nucleic acid sequence encoding the viral structural polypeptide to afford putative identification of that polypeptide, either by manual evaluation of the sequence by one skilled in the art, or by computer-automated sequence comparison and identification using algorithms such as BLAST (see, for example, in “Basic Local Alignment Search Tool”; Altschul S F et al., J. Mol. Biol. 215:403-410, 1993). Accordingly, a substantial portion of a nucleotide sequence comprises enough of the sequence to afford specific identification and/or isolation of a nucleic acid fragment comprising the sequence. For example, a substantial portion of a nucleic acid sequence can include at least about 20%, for example, about 30%, about 40%, about 50%, about 60%, about 70%, about 80%, about 90%, about 95% of the full length nucleic acid sequence.

In some embodiments, the alphavirus srRNA vector is devoid of the entire sequence encoding viral structural proteins, e.g., the alphavirus srRNA vector includes no nucleic acid sequence encoding the viral structural proteins.

In some embodiments, the coding sequence for the PCI includes a coding sequence for a single polypeptide (e.g., monogenic PCI). In some embodiments, the coding sequence for the PCI includes coding sequences for a plurality of polypeptides, e.g., multigenic PCI (e.g., bigenic, trigenic, or tetragenic, etc.). In some embodiments, each of the coding sequences of the plurality of polypeptides is operably linked to a separate promoter sequence. In some embodiments, the coding sequences of the plurality of polypeptides are operably linked to one another within a single open reading frame (e.g., in a polycistronic ORF). In some embodiments, the coding sequence of the polycistronic ORF is operably linked to a promoter sequence. In some embodiments, at least one of the promoter sequences is a subgenomic (sg) promoter. In some embodiments, the sg promoter is a 26S genomic promoter.

In some embodiments, the plurality of polypeptides can be linked to one another directly or indirectly (e.g., via one or more connector sequences). For example, in some embodiments, the plurality of polypeptides can be directly linked to one another, e.g., adjacently to one another. In some embodiments, at least two (e.g., 2, 3, 4, or 5) of the plurality of polypeptides are operably linked to one another by one or more connector sequences. In some embodiments, the length and amino acid composition of the connector sequences can be optimized to vary the orientation, flexibility, and/or proximity of the polypeptides relative to one another to achieve a desired activity or property of the PCI. In some embodiments, a connector sequence of the plurality of connector sequences includes one or more autoproteolytic peptide sequences. Non-limiting examples of autoproteolytic peptide sequences suitable for the methods and compositions of the disclosure include autoproteolytic cleavage sequences derived from calcium-dependent serine endoprotease (furin), porcine teschovirus-1 2A (P2A), foot-and-mouth disease virus (FMDV) 2A (F2A), Equine Rhinitis A Virus (ERAV) 2A (E2A), Thosea asigna virus 2A (T2A), cytoplasmic polyhedrosis virus 2A (BmCPV2A), and Flacherie Virus 2A (BmIFV2A). In some embodiments, at least two of the plurality of polypeptides are operably linked to each other via a P2A autoproteolytic cleavage sequence.

In some embodiments, the coding sequences of the plurality of polypeptides are operably linked to one another by one or more an internal ribosomal entry sites (IRES). Non-limiting examples of IRES suitable for the methods and compositions of the disclosure include viral IRES sequences, cellular IRES sequences, and artificial IRES sequences. Examples of suitable IRES sequences include, but are not limited to, Kaposi's sarcoma-associated herpesvirus (KSHV) IRES, hepatitis virus IRES, Pestivirus IRES, Cripavirus IRES, Rhopalosiphum padi virus IRES, fibroblast growth factor IRES, platelet-derived growth factor IRES, vascular endothelial growth factor IRES, insulin-like growth factor IRES, picornavirus IRES, encephalomyocarditis virus (EMCV) IRES, Pim-1 IRES, p53 IRES, Apaf-1 IRES, TDP2 IRES, L-myc IRES, and c-myc IRES.

### Polypeptide Construct of Interest (PCI)

As described in greater detail below, the polypeptide construct of interest (PCI) can include amino acid sequences of one or more polypeptides. In principle, there are no particular limitations with regard to suitable polypeptides that can be expressed by the srRNA constructs of the disclosure. Exemplary types of polypeptides suitable for the compositions and methods of the disclosure include microbial proteins, viral proteins, bacterial proteins, fungal proteins, mammalian proteins, and any combinations thereof. For example, the PCI can include one or more antigen molecules and/or biotherapeutic molecules, such as cytokines, cytotoxins, chemokines, immunomodulators, pro-apoptotic factors, anti-apoptotic factors, hormones, differentiation factors, dedifferentiation factors, immune cell receptors or reporters, or combinations of any thereof.

In some embodiments, the PCI may include one or more interleukins and interacting proteins, such as G-CSF, GM-CSF, IL-1, IL-10, IL-10-like, IL-11, IL-12, IL-13, IL-14, IL-15, IL-16, IL-17, IL-18, IL-18BP, IL-1-like, IL-1RA, IL-1α, IL-1β, IL-2, IL-20, IL-3, IL-4, IL-5, IL-6, IL-6-like, IL-7, IL-9, IL-21, IL-22, IL-33, IL-37, IL-38, LIF, and OSM. Additional suitable polypeptides include, but are not limited to, interferons (e.g., IFN-α, IFN—P, IFN-7), TNFs (e.g., CD154, LT-β, TNF-α, TNF-β, 4-1BBL, APRIL, CD70, CD153, CD178, GITRL, LIGHT, OX40L, TALL-1, TRAIL, TWEAK, and TRANCE), TGF-β (e.g., TGF-β1, TGF-β2, and TGF-β3), hematopoietins (e.g., Epo, Tpo, Flt-3L, SCF, M-CSF, MSP), chemokines and their receptors (e.g., XCL1, XCL2, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CXCL1, CXCL2, CXCL3, CXCL4, CXCL5, CXCL6, CXCL7, CXCL8, CXCL9, CXCL10, CXCL11, CXCL12, CXCL13, CXCL14, and CX3CL1), immunosuppressive gene products and related transcription factors (e.g., PECAMI, FCGR3A, FOS, NFKB1, JUN, HIF1A, PD-L1, mTOR, STAT5B, and STAT4).

Additional polypeptides suitable for the compositions and methods of the disclosure include, but are not limited to, immunostimulatory gene products (e.g., CD27/CD70, CD40, CD40L, B7.1, BTLA, MAVS, OX40, OX40L, RIG-I, and STING), drug resistant mutants/variants of genes, such as ABCB1, ABCC1, ABCG2, AKT1, ALK, BAFF, BCR-ABL, BRAF, CCND1, cMET, EGFR, ERBB2, ERBB3, ERK2, ESR1, GRB2, KRAS, MDR1, MRP1, NTRK1, PDC4, P-gp, PI3K, PTEN, RET, ROS1, RSK1, RSK2, SHIP, and STK11. Also suitable for the compositions and methods of the disclosure includes sequences encoding viral proteins, in particular spike proteins, fiber proteins, structural proteins, and attachment proteins. In some embodiments, the PCT may include one or more surface-exposed viral antigens, e.g., surface-exposed viral proteins. In some embodiments, the viral protein is a glycoprotein. In some embodiments, the PCT may include a surface glycoprotein. In some embodiments, the viral glycoprotein is a rabies virus glycoprotein. In some embodiments, the viral glycoprotein is an envelope glycoprotein G of a rabies virus (RABV-G).

In some embodiments, the PCI may include amino acid sequences for one or more antibodies or antibody variants (e.g. single chain Fv, bi-specifics, camelids, Fab, and HCAb). In some embodiments, the antibody targets molecules associated with or upregulated in cancers, or molecules associated with infectious disease. In some embodiments, the antibody targets molecules having immunostimulatory function, or having immunosuppressive function.

In some embodiments, the PCI may include amino acid sequences for an enzyme whose deficiency or mutation is associated with diseases or health conditions, such as, for example, agalsidase beta, agalsidase alfa, imiglucerase, taliglucerase alfa, velaglucerase alfa, alglucerase, sebelipase alfa, laronidase, idursulfase, elosulfase alfa, galsulfase, alglucosidase alfa, and CTFR.

In some embodiments, the PCI may include amino acid sequences for a polypeptide selected from antigen molecules, biotherapeutic molecules, or combinations of any thereof. In some embodiments, the PCI may include amino acid sequences for a polypeptide selected from tumor-associated antigens, tumor-specific antigens, neoantigens, and combinations of any thereof. In some embodiments, the PCI can include amino acid sequences for a polypeptide selected from estrogen receptors, intracellular signal transducer enzymes, and human epidermal growth receptors. In some embodiments, the PCI may include amino acid sequences for a biotherapeutic polypeptide selected from immunomodulators, modulators of angiogenesis, modulators of extracellular matrix, modulators of metabolism, neurological modulators, and combinations of any thereof. In some embodiments, the PCI may include amino acid sequences for a cytokine selected from chemokines, interferons, interleukins, lymphokines, and tumor necrosis factors. In some embodiments, the PCI may include amino acid sequences for an interleukins selected from IL-1α, IL-1β, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ and subunits of any thereof. In some embodiments, the PCI may include amino acid sequences a biotherapeutic polypeptide is selected from IL-12A, IL-12B, IL-1RA, and combinations of any thereof.

In some embodiments, the PCI may include one or more antigen polypeptides. As discussed above, in principle, there are no particular limitations with regard to suitable antigen polypeptides that can be expressed by the srRNA constructs of the disclosure. Accordingly, in some embodiments, the PCI can include one or more antigen polypeptides which can be tumor-associated antigens (TAAs), tumor-specific antigens (TSAs), neoantigens, and combinations of any thereof. As will be appreciated by the skilled artisan, TAAs include a molecule, e.g., protein, present on tumor cells and on normal cells, or on many normal cells, but at much lower concentration than on tumor cells. In contrast, TSAs generally include a molecule, e.g., protein which is present on tumor cells but absent from normal cells. The tumor-associated antigen can be an antigen associated with a cancer cell, e.g., a breast cancer cell, a B cell lymphoma, a pancreatic cancer, a Hodgkin lymphoma cell, an ovarian cancer cell, a prostate cancer cell, a mesothelioma, a lung cancer cell, a non-Hodgkin B-cell lymphoma (B-NHL) cell, an ovarian cancer cell, a prostate cancer cell, a mesothelioma cell, a melanoma cell, a chronic lymphocytic leukemia cell, an acute lymphocytic leukemia cell, a neuroblastoma cell, a glioma, a glioblastoma, a colorectal cancer cell, etc. It will also be understood that in some cases, a tumor-associated antigen may also be expressed by a non-cancerous cell.

In some embodiments, the one or more antigen polypeptides includes estrogen receptors, intracellular signal transducer enzymes, and human epidermal growth receptors. In some embodiments, the one or more antigen polypeptides is selected from the group consisting of ESR1, PI3K, HER2, HER3, variants of any thereof, and combinations of any thereof. In some embodiments, the coding sequence for an ESR1 variant includes one or more molecular alterations that promote ligand-independent receptor activities. In some embodiments, the one or more molecular alterations comprises an activating mutation selected from the group consisting of K303R, E380Q, Y537C, Y537S, Y537N, and D538G. In some embodiments, the coding sequence for the PI3K variant includes one or more molecular alterations that promote ligand-independent receptor activities. In some embodiments, the one or more molecular alterations comprises an activating mutation selected from the group consisting of E542K, E545K, H1047L, and H1047R. In some embodiments, the HER2 variant includes a coding sequence for the extracellular domain and transmembrane domain. In some embodiments, the HER3 variant includes a coding sequence for a kinase-inactive HER3. In some embodiments, the coding sequence for the PCI includes, in 5′- to 3′-direction: a) a coding sequence for a variant of PI3K comprising one or more activating molecular alterations selected from E542K, H1047L, E545K, and H1047R; b) a coding sequence for an autoproteolytic peptide P2A; c) a coding sequence for a variant of HER2 comprising its extracellular domain and transmembrane domain; d) a coding sequence for an autoproteolytic peptide P2A; e) a coding sequence for a kinase-inactive variant of HER3; f) a coding sequence for an internal ribosomal entry site (IRES); and g) a coding sequence for a variant of ESR1 comprising one or more activating molecular alterations selected from Y537C, E380Q, K303R, Y537S, D538G, and Y537N.

In some embodiments, the PCI includes one or more biotherapeutic polypeptides. Generally, there are no particular limitations with regard to suitable biotherapeutic polypeptides that can be expressed by the srRNA constructs of the disclosure. Examples of biotherapeutic polypeptides suitable for the compositions and methods of the disclosure include, but are not limited to, immunomodulators, modulators of angiogenesis, modulators of extracellular matrix, modulators of metabolism, neurological modulators, and combinations of any thereof. In some embodiments, the PCI includes one or more cytokines. Non-limiting examples of cytokines include from chemokines, interferons, interleukins, lymphokines, and tumor necrosis factors. In some embodiments, the PCI includes one or more interleukins. Suitable interleukins include IL-1α, IL-1β, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ, and subunits of any thereof. In some embodiments, the one or more biotherapeutic polypeptides is selected from a p35 subunit of interleukin 12 (p35 or IL-12A), p40 subunit of interleukin 12 (p40 or IL-12B), an interleukin-1 receptor antagonist (IL-1RA), variants of any thereof, and combinations of any thereof. In some embodiments, the polypeptide construct of interest includes, in N-terminus to C-terminus direction: a) an IL-12A polypeptide, an IL-12B polypeptide, and an IL-1RA polypeptide; or b) an IL-1RA polypeptide, an IL-12B polypeptide, and an IL-12A polypeptide; wherein the IL-12A, IL-12B, and IL-1RA polypeptides are operably linked to one another by one or more autoproteolytic cleavage sequences or internal ribosomal entry sites.

In some embodiments, step (a) of the methods described herein includes providing a plurality of srRNA expression constructs each including a coding sequence for a variant of the PCI. The term “variant” of a PCI refers to a polypeptide construct in which one or more one or more molecular alterations (e.g., amino acid deletions, substitutions, insertion, duplications, and/or mutations) are present as compared to the amino acid sequence of original PCT. The term variant also encompasses frameshift variants and splice variants. In some embodiments, the providing in (a) includes: (i) obtaining an alphavirus srRNA expression vector, wherein at least a portion of encoding sequence for the alphavirus structural proteins has been replaced with a coding sequence for a polypeptide construct of interest (PCI); and (ii) generating a plurality of srRNA expression constructs each including a coding sequence for a variant of the PCI.

In some embodiments, step (a) of the methods described herein includes providing a plurality of srRNA expression constructs each including a coding sequence for a variant of the PCI, wherein the providing in (a) includes: (i) obtaining coding sequences for a plurality of variants of a polypeptide construct of interest (PCI); and (ii) generating a plurality of srRNA expression constructs each including a coding sequence for a PCI variant of the plurality of PCI variants from (a) operably inserted into an alphavirus srRNA vector, wherein at least a portion of the encoding sequence for the alphavirus structural proteins has been replaced with the coding sequence of the PCI variant.

In some embodiments of the methods described herein, a PCI variant of the plurality of PCI variants includes one or more molecular alterations. Exemplary types of molecular alterations in the PCI variant can be one or more of deletions, substitutions, insertion, duplications, mutations, frameshift variants, splice variants, and combinations of any thereof.

In some embodiments of the methods described herein, the one or more molecular alterations are configured into a plurality of alteration cassettes. In some embodiments, the plurality of alteration cassettes are arranged in tandem along the length of the PCI sequence. In some embodiments, the length and amino acid composition of the alteration cassettes can be optimized to achieve a desired activity or property of the PCI or PCI variant. In some embodiments, an alteration cassette of the plurality of alteration cassettes includes a single-chain polypeptide sequence comprising about 1 to about 30 amino acid residues (e.g., 2, 3, 4, 5, 6, 7, 8, 9, 10, 11, 12, 13, 14, 15, 16, 17, 18, 19, 20, etc. amino acid residues). In some embodiments, an alteration cassette of the plurality of alteration cassettes includes about 2 to about 50 amino acid residues, such as about 5 to about 45, about 10 to about 40, about 15 to about 30, about 20 to about 50, about 2 to about 30, about 3 to about 25, about 4 to about 20, about 5 to about 15, about 6 to about 10, about 3 to about 15, about 4 to about 10, about 5 to about 30, about 2 to about 5, about 3 to about 5, about 4 to about 8 amino acid residues. In some embodiments, an alteration cassette of the plurality of alteration cassettes includes 31 amino acid residues. In some embodiments, an alteration cassette of the plurality of alteration cassettes includes one, two, three, four, five, or more molecular alterations.

In some embodiments, the coding sequence of the PCI is refactored and/or optimized for a desired property, such as increased stability, potency, and expression (e.g., translation efficiency), which in turns can maximize the impact of producing, delivering, and administering biotherapeutics. For example, in some embodiments, the coding sequence of the PCI is optimized for expression at a level higher than the expression level of a reference coding sequence, for example, 20% higher, 30% higher, 40% higher, 50% higher, 60% higher, 70% higher, 80% higher, 90% higher, or 95% higher than a reference coding sequence. In some embodiments, the reference coding sequence is a wild-type non-optimized sequence. With respect to sequence-optimization of nucleotide sequences, degeneracy of the genetic code provides the possibility to substitute at least one base of the protein encoding sequence of a gene with a different base without causing the amino acid sequence of the polypeptide produced from the gene to be changed. Hence, the nucleic acid constructs of the present disclosure can also have any base sequence that has been changed from any polynucleotide sequence disclosed herein by substitution in accordance with degeneracy of the genetic code. References describing codon usage are readily publicly available. In some embodiments, polynucleotide sequence variants can be produced for a variety of reasons, e.g., to optimize expression for a particular host (e.g., changing codon usage in the alphavirus mRNA to those preferred by other organisms such as human, non-human primates, hamster, mice, or monkey). Accordingly, in some embodiments, the coding sequence of the PCI is optimized for expression at a level higher than the expression level of a reference coding sequence, such as, for example, a coding sequence that has not been codon-optimized in a target host cell through the use of codons optimized for expression. In some embodiments, the codon-optimized sequence of the PCI results in an increased expression level by at least 30%, at least 40%, at least 50%, at least 60%, at least 70%, at least 80%, at least 90%, at least 95%, or at least 100% compared to a reference coding sequence that has not been codon-optimized. In some embodiments, the codon-optimized sequence of the PCI results in an increased expression level by at least 2-fold, at least 3-fold, at least 4-fold, or at least 5-fold compared to a reference coding sequence that has not been codon-optimized.

In some embodiments, the coding sequence of the PCI is optimized for enhanced RNA stability and/or expression. The stability of RNA generally relates to the “half-life” of RNA. “Half-life” relates to the period of time which is needed to eliminate half of the activity, amount, or number of molecules. In the context of the present disclosure, the half-life of an RNA is indicative for the stability of said RNA. The half-life of RNA may influence the “duration of expression” of the RNA. Several methodologies and techniques useful for evaluation of RNA stability are known, including various in silico methodologies and/or empirical stress-testing of storage of replicons, e.g., self-replicating RNAs, with different PCI codon usage, and its effects on srRNA potency (e.g., examine dsRNA in cells following transfection) and gene expression. Additional information in this regard can be found in, for example, Wayment-Steele, H. et al. (2021). Cold Spring Harbor Laboratory (doi.org/10.1101/2020.08.22.262931). Further information regarding principles, strategies, and methods for use in enhancing RNA stability can be found in, for example, Leppek K. et al., Combinatorial optimization of mRNA structure, stability, and translation for RNA-based therapeutics. bioRxiv. (Preprint). Mar. 30, 2021. doi: 10.1101/2021.03.29.437587.

In some embodiments, the plurality of alteration cassettes are operably linked to one another by one or more linkers. In some embodiments, a linker of the one or more linkers includes a synthetic compound linker or a peptide linker. In some embodiments, the linker can be a synthetic compound linker such as, for example, a chemical cross-linking agent. Non-limiting examples of suitable cross-linking agents that are available on the market include N-hydroxysuccinimide (NHS), disuccinimidylsuberate (DSS), bis(sulfosuccinimidyl)suberate (BS3), dithiobis(succinimidylpropionate) (DSP), dithiobis(sulfosuccinimidylpropionate) (DTSSP), ethyleneglycol bis(succinimidylsuccinate) (EGS), ethyleneglycol bis(sulfosuccinimidylsuccinate) (sulfo-EGS), disuccinimidyl tartrate (DST), disulfosuccinimidyl tartrate (sulfo-DST), bis[2-(succinimidooxycarbonyloxy)ethyl]sulfone (BSOCOES), and bis[2-(sulfosuccinimidooxycarbonyloxy)ethyl]sulfone (sulfo-BSOCOES).

In some embodiments, the linker can be a peptide linker, which joins together two adjacent alteration cassettes, as described herein. In some embodiments, the length and amino acid composition of the peptide linker sequence can be optimized to vary the orientation, flexibility, and/or proximity of the alteration cassettes relative to one another to achieve a desired activity or property of the PCI or PCI variant.

In some embodiments, a polypeptide linker includes a single-chain polypeptide sequence comprising about 1 to about 30 amino acid residues (e.g., 2, 3, 4, 5, 6, 7, 8, 9, 10, 11, 12, 13, 14, 15, 16, 17, 18, 19, 20, etc. amino acid residues). In some embodiments, a linker sequence includes about 2 to 30, about 3 to 25, about 4 to 20, about 5 to 15, about 6 to 10, about 3 to 15, about 4 to 10, about 5 to 30, about 2 to 5, about 3 to 5, about 4 to 8 amino acid residues.

In some embodiments, the length and amino acid composition of the linker polypeptide sequence can be optimized to vary the orientation, flexibility, and/or proximity of the alteration cassettes relative to one another to achieve a desired activity or property of the PCI. In some embodiments, the orientation, flexibility, and/or proximity of the alteration cassettes relative to one another can be varied as a “tuning” tool to achieve a tuning effect that would enhance or reduce the activity of the PCI or PCI variant. In certain embodiments, the linker contains only glycine and/or serine residues (e.g., glycine-serine linker). Examples of such polypeptide linkers include: Gly, Ser; Gly Ser; Gly Gly Ser; Ser Gly Gly; Gly Gly Gly Ser; Ser Gly Gly Gly; Gly Gly Gly Gly Ser; Ser Gly Gly Gly Gly; Gly Gly Gly Gly Gly Ser; Ser Gly Gly Gly Gly Gly; Gly Gly Gly Gly Gly Gly Ser; Ser Gly Gly Gly Gly Gly Gly; (Gly Gly Gly Gly Ser)n, wherein n is an integer of one or more; and (Ser Gly Gly Gly Gly)n, wherein n is an integer of one or more. In some embodiments, the polypeptide linkers are modified such that the amino acid sequence Gly Ser Gly (GSG) (that occurs at the junction of traditional Gly/Ser linker polypeptide repeats) is not present. In some embodiments, the peptide linker includes an amino acid sequence selected from the group consisting of AAY, EAAAK (SEQ ID NO: 1), RVRR (SEQ ID NO: 2), GGGGS (SEQ ID NO: 3), and GPGPG (SEQ ID NO: 4).

Analysis of Level and/or Functionality of the PCIs

As described above, the methods of the disclosure include a process of analyzing level and/or functionality of the PCIs that are expressed from the plurality of srRNA expression constructs to identify one or more candidate PCIs having a defined property (see, e.g., FIG. 1). The analysis of level and/or functionality of the PCIs can be carried out in vitro, in vivo, or ex vivo. In some embodiments, the analysis of level and/or functionality of the PCIs can include one of more analytical techniques. Examples of suitable analytical techniques include, but are not limited to, immunoblotting analysis, fluorescence flow cytometry analysis, enzyme-linked immunoassay analysis, immunogenicity analysis, bioactivity analysis, and efficacy in a disease model.

Generation of a Combinatorial Collection of Delivery Systems Including srRNA Expression Constructs

As described above, the methods of the disclosure include a process of incorporating the srRNA expression constructs capable of expressing the candidate PCIs identified in (b) with at least one delivery vehicle to create a combinatorial collection of delivery systems. Exemplary delivery vehicles suitable for the methods and compositions of the disclosure include, but are not limited to, physiologic buffers, liposomes, lipid-based nanoparticles (LNPs), polymer nanoparticles, viral replicon particles (VRPs), microspheres, immune stimulating complex (ISCOM), and conjugates of bioactive ligands which can facilitate delivery and/or enhance the immune response. These compounds are readily available to one skilled in the art; for example, see Liposomes: A Practical Approach, RCP New Ed, TRL press (1990). Adjuvants other than liposomes and the like are also used and are known in the art. Adjuvants may protect the antigen (e.g., srRNA construct) from rapid dispersal by sequestering it in a local deposit, or they may contain substances that stimulate the host to secrete factors that are chemotactic for macrophages and other components of the immune system. An appropriate selection can be made by those skilled in the art, for example, from those described below.

In some embodiments, the delivery systems can include one or more of the following: physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof.

Exemplary types of lipids suitable for the delivery systems described herein include cationic lipids, ionizable cationic lipids, anionic lipids, neutral lipids, and combinations thereof.

In some embodiments, the LNP of the disclosure can include one or more ionizable lipids. Exemplary ionizable lipids suitable for the compositions and methods of the disclosure includes those described in PCT publications WO2020252589A1 and WO2021000041A1, and Love K. T. et al., Proc Natl Acad Sci USA, Feb. 2, 2010 107 (5) 1864-1869, which are incorporated by reference herein in their entirety.

Accordingly, in some embodiments, the LNP of the disclosure includes one or more lipid compounds described in Love K. T. et al., 2010 supra, such as C16-96, C14-110, and C12-200. In some embodiments, the LNP includes an ionizable cationic lipid selected from the group consisting of ALC-0315, C12-200, LN16, MC3, MD1, SM-102, and a combination of any thereof. In some embodiments, the LNP of the disclosure includes C12-200.

In some embodiments, the LNP of the disclosure includes one or more cationic lipids. Suitable cationic lipids include, but are not limited to, 98N12-5, C12-200, C14-PEG2000, DLin-KC2-DMA (KC2), DLin-MC3-DMA (MC3), XTC, MD1, and 7C1.

In some embodiments, the LNP of the disclosure includes one or more neutral lipids. As described above, neural lipids, also known as “structural lipids” or “helper lipids” can also be incorporated into lipid formulations and lipid particles in some embodiments. The lipid formulations and lipid particles can include one or more structural lipids at about 10 to 40 Mol % of the composition. Suitable structural lipids support the formation of particles during manufacture. Structural lipids refer to any one of a number of lipid species that exist in either in an anionic, uncharged or neutral zwitterionic form at physiological pH. Representative structural lipids include diacylphosphatidylcholines, diacylphosphatidylethanolamines, diacylphosphatidylglycerols, ceramides, sphingomyelins, dihydrosphingomyelins, cephalins, and cerebrosides.

Exemplary structural lipids include zwitterionic lipids, for example, distearoylphosphatidylcholine (DSPC), dioleoylphosphatidylcholine (DOPC), dipalmitoylphosphatidylcholine (DPPC), dioleoyl-phosphatidylethanolamine (DOPE), palmitoyloleoylphosphatidylcholine (POPC), palmitoyloleoyl-phosphatidylethanolamine (POPE) and dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanol amine (SOPE), and 1,2-dielaidoyl-sn-glycero-3-phophoethanolamine (trans DOPE).

In another embodiment, the structural lipid can be any lipid that is negatively charged at physiological pH. These lipids include phosphatidylglycerols such as dioleoylphosphatidylglycerol (DOPG), dipalmitoylphosphatidylglycerol (DPPG), palmitoyloleyolphosphatidylglycerol (POPG), cardiolipin, phosphatidylinositol, diacylphosphatidylserine, diacylphosphatidic acid, and other anionic modifying groups joined to neutral lipids. Other suitable structural lipids include glycolipids (e.g., monosialoganglioside GM1).

Non-limiting neutral lipids suitable for the compositions and methods of the disclosure include DPSC, DPPC, POPC, DOPE, and SM. In some embodiments, the LNP of the disclosure includes one or more ionizable lipid compounds described in PCT publications WO2020252589A1 and WO2021000041A1, which are incorporated by reference herein in their entirety.

In some embodiments, the LNP of the disclosure includes at least one lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG).

In some embodiments where the delivery systems described herein include an LNP, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles (LNPs) have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

Stabilizing agents can be included in lipid formulations embodiments to ensure integrity of the mixtures. Stabilizing agents are a class of molecules which disrupt or help form the hydrophobic-hydrophilic interactions among molecules. Suitable Stabilizing agents include, but are not limited to, polysorbate 80 (also known as Tween 80, IUPAC name 2-[2-[3,4-bis(2-hydroxyethoxy)oxolan-2-yl]-2-(2-hydroxyethoxy)ethoxy]ethyl octadec-9-enoate), Myrj52 (Polyoxyethylene (40) stearate), and Brij™ S10 (Polyoxyethylene (10) stearyl ether). Polyethylene glycol conjugated lipids may also be used. The stabilizing agents may be used alone or in combinations with each other.

In some embodiments, the stabilizing agents comprises about 0.1 to 3 Mol % of the overall lipid mixture. In some embodiments, the stabilizing agents comprise about 0.5 to 2.5 Mol % of the overall lipid mixture. In some embodiments, the stabilizing agent is present at greater than 2.5 Mol %. In some embodiments the stabilizing agent is present at 5 Mol %. In some embodiments the stabilizing agent is present at 10 Mol %. In some embodiments, the stabilizing agent is about 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, and so forth. In other embodiments, the stabilizing agent is 2.6-10 Mol % of the lipid mixture. In other embodiments, the stabilizing agents is present at greater than 10 Mol % of the lipid mixture.

Steroids can also be included in the lipid compositions for certain applications, and lipid particles made therefrom include sterols, such as cholesterol and phytosterol.

### In Vivo or Ex Vivo Assessment of Pharmacodynamic Effects

As described above, some embodiments of the methods of the disclosure include a process of analyzing the delivery systems for their capability to confer at least one pharmacodynamic effect in a subject to identify a srRNA expression construct capable of conferring a desired pharmacodynamic effect. In some embodiments, the analysis of the delivery systems for their capacity to confer at least one pharmacodynamic effects in step (d) is carried out in vivo or ex vivo. Examples of pharmacodynamic effects that can be analyzed include: immunogenicity effect (e.g., eliciting an immune response in vivo), a biomarker response, a therapeutic effect, a prophylactic effect, a desired effect, an undesired effect, an adverse effect, and effect in a disease model. In some embodiments, the assessment of pharmacodynamic effects includes assessing induction of an immune response in vivo (see, e.g., Examples 1-4). In some embodiments, the assessment of pharmacodynamic effects includes assessing induction of cytokine pathways that can potentiate an immune response and prevent angiogenesis and metastasis (see, e.g., Examples 5-7).

In some embodiments of the methods described herein, the recombinant alphavirus srRNA is of a virus belonging to the Alphavirus genus of the Togaviridae family. In some embodiments, the recombinant alphavirus srRNA is of an alphavirus belonging to the VEEV/EEEV group, or the SFV group, or the SINV group. In some embodiments, the alphavirus is Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu virus (NDUV), Madariaga virus (MADV), or Buggy Creek virus. In some embodiments, the alphavirus is VEEV, EEEV), CHIKV, or SINV. In some embodiments, the alphavirus is VEEV. In some embodiments, the alphavirus is EEEV. In some embodiments, the alphavirus is CHIKV. In some embodiments, the alphavirus is SINV.

Suitable wild-type alphavirus sequences are well-known and are available from sequence depositories, such as the American Type Culture Collection, Rockville, Md. Representative examples of suitable alphaviruses include Aura (ATCC VR-368), Bebaru virus (ATCC VR-600, ATCC VR-1240), Cabassou (ATCC VR-922), Chikungunya virus (ATCC VR-64, ATCC VR-1241), Eastern equine encephalomyelitis virus (ATCC VR-65, ATCC VR-1242), Fort Morgan (ATCC VR-924), Getah virus (ATCC VR-369, ATCC VR-1243), Kyzylagach (ATCC VR-927), Mayaro (ATCC VR-66), Mayaro virus (ATCC VR-1277), Middleburg (ATCC VR-370), Mucambo virus (ATCC VR-580, ATCC VR-1244), Ndumu (ATCC VR-371), Pixuna virus (ATCC VR-372, ATCC VR-1245), Ross River virus (ATCC VR-373, ATCC VR-1246), Semliki Forest (ATCC VR-67, ATCC VR-1247), Sindbis virus (ATCC VR-68, ATCC VR-1248), Tonate (ATCC VR-925), Triniti (ATCC VR-469), Una (ATCC VR-374), Venezuelan equine encephalomyelitis (ATCC VR-69, ATCC VR-923, ATCC VR-1250 ATCC VR-1249, ATCC VR-532), Western equine encephalomyelitis (ATCC VR-70, ATCC VR-1251, ATCC VR-622, ATCC VR-1252), Whataroa (ATCC VR-926), and Y-62-33 (ATCC VR-375).

In some embodiments, the alphavirus is Chikungunya virus (CHIKV). Non-limiting examples of CHIKV strains suitable for the compositions and methods of the disclosure include CHIKV S27, CHIKV LR2006-OPY-1, CHIKV YO123223, CHIKV DRDE, CHIKV 37997, CHIKV 99653, CHIKV Ag41855, and Nagpur (India) 653496 strain. Virulent and avirulent CHIKV strains are both suitable. Additional examples of CHIKV strains suitable for the compositions and methods of the disclosure include but are not limited to those described in Afreen et al. Microbiol. Immunol. 2014, 58:688-696, Lanciotti and Lambert ASTMH 2016, 94(4):800-803 and Langsjoen et al. mBio. 2018, 9(2):e02449-17. In some embodiments, the modified CHIKV genome or replicon, e.g., self-replicating RNA, is derived from CHIKV strain S27. In some embodiments, the modified CHIKV genome or srRNA is derived from CHIKV strain DRDE. In some embodiments, the modified CHIKV genome or srRNA is derived from CHIKV strain DRDE-06. In some embodiments, the modified CHIKV genome or srRNA is derived from CHIKV strain DRDE-07.

In some embodiments, the alphavirus is Eastern Equine Encephalitis virus (EEEV) or Madariaga virus (MADV). Non-limiting examples of EEEV and MADV strains suitable for the compositions and methods of the disclosure include EEEV 792138, 783372, BeAn5122, BeAr300851, BeAr436087, C-49, FL91-4679, FL93-939, GML903836, MP-9, PE6, and V105-00210. Virulent and avirulent EEEV strains are both suitable. Additional suitable EEEV strains include, but are not limited to those described in the Virus Pathogen Resource website (ViPR; which is publicly available at www.viprbrc.org/brc/vipr_genome_search.spg?method=SubmitForm&blockId=868&decorator=toga). In some embodiments, the modified EEEV genome or replicon, e.g., self-replicating RNA, is derived from EEEV strain FL93-939. Additional suitable MADV strains include, but are not limited to those described in Arrigo et al., J. Virology, January 2010, 1014-1025, which is herein incorporated by reference. In some embodiments, the modified MADV genome or replicon, e.g., self-replicating RNA, is derived from MADV strain BeAr300851.

In some embodiments, the alphavirus is Sindbis virus (SINV). In some embodiments, the modified genome or replicon, e.g., self-replicating RNA, is of a SINV strain. Non-limiting examples of SINV strains suitable for the compositions and methods of the disclosure include SINV strain AR339, AR86, and Girdwood. Examples of SINV strains suitable for the compositions and methods of the disclosure include, but are not limited to those described in Sammels et al. J. Gen. Virol. 1999, 80(3):739-748, Lundstrom and Pfeffer Vector Borne Zoonotic Dis. 2010, 10(9):889-907, Sigei et al. Arch. of Virol. 2018, 163:2465-2469 and Ling et al. J. Virol. 2019, 93:e00620-19. Additional suitable SINV strains include, but are not limited to those described in the Virus Pathogen Resource website (ViPR). Virulent and avirulent SINV strains are both suitable. In some embodiments, the modified genome or srRNA is of a SINV strain Girdwood. In some embodiments, the modified genome or srRNA is of a SINV strain AR86. In some embodiments, the modified SINV genome or srRNA is derived from SINV strain Girdwood. In some embodiments, the modified SINV genome or srRNA is derived from SINV strain AR86. In some embodiments, the at least one heterologous nsP or portion thereof of the modified genome or srRNA is derived from a SINV strain AR86. In some embodiments, the at least one heterologous nsP or portion thereof is nsP1, nsP3, nsP4, or a portion of any thereof, or a combination of any of the foregoing. In some embodiments, the modified genome or srRNA is of a SINV strain AR86.

In some embodiments, the alphavirus is Western Equine Encephalitis virus (WEEV). Non-limiting examples of WEEV strains suitable for the compositions and methods of the disclosure include WEEV California, McMillan, IMP181, Imperial, Imperial181, IMPR441, 71V-1658, AG80-646, BFS932, COA592, EP-6, E1416, BFS1703, BFS2005, BSF3060, BSF09997, CHLV53, KERN5547, 85452NM, Montana-64, 58-122, and TBT-235. Additional examples of WEEV strains suitable for the compositions and methods of the disclosure include 5614, 93A27, 93A30, 93A38, 93A79, B628(Cl 15), CBA87, CNTR34, C0921356, Fleming, Lake43, PV012357A, PV02808A, PV72102, R02PV001807A, R02PV002957B, R02PV003422B, R05PV003422B, ROPV003814A and ROPV00384A. Virulent and avirulent WEEV strains are both suitable. Additional suitable WEEV strains include, but are not limited to those described in Bergren N A et al., J. Virol. 88(16): 9260-9267, August 2014, and in the Virus Pathogen Resource website (ViPR). In some embodiments, the modified WEEV genome or srRNA is derived from WEEV strain Imperia.

In some embodiments of the methods described herein, the srRNA expression vector is identified as having an immune-inducing activity suitable for a prophylactic use and/or a therapeutic use. In some embodiments, the srRNA expression vector is identified as having an immune-inducing activity suitable for a prophylactic use. In some embodiments, the srRNA expression vector is identified as having an immune-inducing activity suitable for a therapeutic use.

### COMPOSITIONS OF THE DISCLOSURE

### Nucleic Acid Constructs

As described in greater detail below, one aspect of the present disclosure relates to nucleic acid constructs including a nucleic acid sequence encoding a srRNA construct as described herein. In some embodiments, the sequence encoding a srRNA construct can be operably linked, e.g., placed under the control of elements required for expression (e.g., promoter sequences), which allow expression of the srRNA construct in a host cell, in a subject, or in an ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR § 1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 2 kb and 50 kb in length, for example between about 5 kb and about 40 kb, between about 5 kb and about 30 kb, between about 5 kb and about 20 kb, or between about 10 kb and about 50 kb, for example between about 15 kb to 30 kb, between about 20 kb and about 50 kb, between about 20 kb and about 40 kb, between about 5 kb and about 25 kb, or between about 30 kb and about 50 kb. In some embodiments, the nucleic acid molecules are at least 6 kb in length. In some embodiments, the nucleic acid molecules are between about 6 kb and about 20 kb. In some embodiments, the nucleic acid constructs (e.g., vectors or srRNA constructs) of the disclosure generally have a length of at least about 2 kb. For example, the nucleic acid constructs (e.g., vectors or srRNAs) can have a length of at least about 2 kb, at least about 3 kb, at least about 4 kb, at least about 5 kb, at least about 6 kb, at least about 7 kb, at least about 8 kb, at least about 9 kb, at least about 10 kb, at least about 11 kb, at least about 12 kb or more than 12 kb. In some embodiments, the nucleic acid constructs (e.g., vectors or srRNAs) can have a length of about 4 kb to about 20 kb, about 4 kb to about 18 kb, about 5 kb to about 16 kb, about 6 kb to about 14 kb, about 7 kb to about 12 kb, about 8 kb to about 16 kb, about 9 kb to about 14 kb, about 10 kb to about 18 kb, about 11 kb to about 16 kb, about 5 kb to about 18 kb, about 6 kb to about 20 kb, about 5 kb to about 10 kb, about 5 kb to about 8 kb, about 5 kb to about 7 kb, about 5 kb to about 6 kb, about 6 kb to about 12 kb, about 6 kb to about 11 kb, about 6 kb to about 10 kb, about 6 kb to about 9 kb, about 6 kb to about 8 kb, about 6 kb to about 7 kb, about 7 kb to about 11 kb, about 7 kb to about 10 kb, about 7 kb to about 9 kb, about 7 kb to about 8 kb, about 8 kb to about 11 kb, about 8 kb to about 10 kb, about 8 kb to about 9 kb, about 9 kb to about 11 kb, about 9 kb to about 10 kb, or about 10 kb to about 11 kb. In some embodiments, the nucleic acid constructs (e.g., vectors or srRNAs) can have a length of about 6 kb to about 14 kb. In some embodiments, the nucleic acid constructs (e.g., vectors or srRNAs) can have a length of about 6 kb to about 16 kb.

In some embodiments, the nucleic acid sequence encoding the srRNA construct is incorporated into an expression cassette or an expression vector. It will be understood that an expression cassette generally includes a construct of genetic material that contains the coding sequences for the srRNA and enough regulatory information to direct proper transcription and/or translation of the coding sequences in a recipient cell, in vivo and/or ex vivo. Generally, the expression cassette may be inserted into a vector for targeting to a desired host cell and/or into a subject or individual. In some embodiments, the term expression cassette can be used interchangeably with the term “expression construct.” As such, in some embodiments, an expression cassette of the disclosure include a coding sequence for the srRNA as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon (e.g., srRNA), such as a plasmid, phage, or cosmid, into which another DNA segment may be inserted so as to bring about the replication of the inserted segment. In some embodiments, the expression vector can be an integrating vector.

The molecular techniques and methods by which the nucleic acid constructs of the disclosure can be assembled and characterized are described more fully in the Examples herein of the present application.

In some embodiments, the nucleic acid molecules are recombinant nucleic acid molecules. As described above, the term recombinant nucleic acid molecule means any nucleic acid molecule (e.g. DNA, RNA), that is, or results, however indirect, from human manipulation. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule: 1) has been synthesized or modified in vitro, for example, using chemical or enzymatic techniques (for example, by use of chemical nucleic acid synthesis, or by use of enzymes for the replication, polymerization, exonucleolytic digestion, endonucleolytic digestion, ligation, reverse transcription, transcription, base modification (including, e.g., methylation), or recombination (including homologous and site-specific recombination) of nucleic acid molecules; 2) includes conjoined nucleotide sequences that are not conjoined in nature; 3) has been engineered using molecular cloning techniques such that it lacks one or more nucleotides with respect to the naturally occurring nucleotide sequence; and/or 4) has been manipulated using molecular cloning techniques such that it has one or more sequence changes or rearrangements with respect to the naturally occurring nucleotide sequence.

In some embodiments, the nucleic acid molecules disclosed herein are produced using recombinant DNA technology (e.g., polymerase chain reaction (PCR) amplification, cloning, etc.) or chemical synthesis. Nucleic acid molecules as disclosed herein include natural nucleic acid molecules and homologs thereof, including, but not limited to, natural allelic variants and modified nucleic acid molecules in which one or more nucleotide residues have been inserted, deleted, and/or substituted, in such a manner that such modifications provide the desired property in effecting a biological activity as described herein.

One skilled in the art will appreciate that nucleic acid molecules, including variants of a naturally-occurring nucleic acid sequence, can be produced using a number of methods known to those skilled in the art (see, for example, Sambrook et al., In: Molecular Cloning, A Laboratory Manual, 2nd Edition, Cold Spring Harbor Press, Cold Spring Harbor, N.Y. (1989)). The sequence of a nucleic acid molecule can be modified with respect to a naturally-occurring sequence from which it is derived using a variety of techniques including, but not limited to, classic mutagenesis techniques and recombinant DNA techniques, such as but not limited to site-directed mutagenesis, chemical treatment of a nucleic acid molecule to induce mutations, restriction enzyme cleavage of a nucleic acid fragment, ligation of nucleic acid fragments, PCR amplification and/or mutagenesis of selected regions of a nucleic acid sequence, recombinational cloning, and chemical synthesis, including chemical synthesis of oligonucleotide mixtures and ligation of mixture groups to “build” a mixture of nucleic acid molecules, and combinations thereof. Nucleic acid molecule homologs can be selected from a mixture of modified nucleic acid molecules by screening for the function of the protein or the replicon, e.g., self-replicating RNA, encoded by the nucleic acid molecule and/or by hybridization with a wild-type gene or fragment thereof, or by PCR using primers having homology to a target or wild-type nucleic acid molecule or sequence.

### Recombinant Cells

As described in greater detail below, one aspect of the present disclosure relates to recombinant cells that have been engineered to include a nucleic acid construct as described herein and/or include (e.g., express) a srRNA construct as described herein. In some embodiments, a nucleic acid construct and/or srRNA construct of the present disclosure can be introduced into a host cell to produce a recombinant cell containing the nucleic acid construct and/or srRNA construct. Accordingly, prokaryotic or eukaryotic cells that contain a srRNA construct as described herein and/or a nucleic acid construct encoding a srRNA construct as described herein are also features of the disclosure. Introduction of the srRNA constructs and nucleic acid constructs of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, direct micro-injection, nanoparticle-mediated nucleic acid delivery, and the like.

Accordingly, some embodiments of the disclosure relate to recombinant cells, for example, recombinant eukaryotic cells, e.g., insect cells or animal cells that include a srRNA construct and/or a nucleic acid construct described herein. In some embodiments, the recombinant cells include a nucleic acid construct, wherein the nucleic acid construct can be stably integrated in the host genome, or can be episomally replicating, or present in the recombinant host cell as a mini-circle expression vector for a stable or transient expression. Accordingly, in some embodiments of the disclosure, the nucleic acid construct is maintained and replicated in the recombinant host cell as an episomal unit. In some embodiments, the nucleic acid construct is stably integrated into the genome of the recombinant cell. Stable integration can be completed using classical random genomic recombination techniques or with more precise genome-editing techniques such as using guide RNA directed CRISPR/Cas9 or TALEN genome editing. In some embodiments, the nucleic acid construct present in the recombinant host cell as a mini-circle expression vector for a stable or transient expression.

Host cells can be either untransformed cells or cells that have already been transfected with at least one nucleic acid molecule. Accordingly, in some embodiments, host cells can be genetically engineered (e.g., transduced or transformed or transfected) with at least one nucleic acid molecule.

Suitable host cells for cloning or expression of the polypeptides of interest as described herein include prokaryotic or eukaryotic cells described herein. Accordingly, in some embodiments, the recombinant cell of the disclosure is a prokaryotic cell, such as the bacterium E. coli, or a eukaryotic cell, such as an insect cell (e.g., a mosquito cell or a Sf21 cell), or mammalian cells (e.g., COS cells, NIH 3T3 cells, or HeLa cells). In some embodiments, the cell is in vivo, for example, a recombinant cell in a living body, e.g., cell of a transgenic subject. In some embodiments, the subject is a vertebrate animal or an invertebrate animal. In some embodiments, the subject is an insect. In some embodiments, the subject is a mammalian subject. In some embodiments, the mammalian subject is a human subject. In some embodiments, the cell is ex vivo, e.g., has been extracted, as an individual cell or as part of an organ or tissue, from a living body or organism for a treatment or procedure, and then returned to the living body or organism. In some embodiments, the cell is in vitro, e.g., is obtained from a repository.

In some embodiments of the disclosure, the recombinant cell of the disclosure is a eukaryotic cell. In some embodiments, the recombinant cell is an animal cell. In some embodiments, the animal cell is a vertebrate animal cell or an invertebrate animal cell. In some embodiments, the recombinant animal cell is a mammalian cell. In some embodiments, non-liming examples of a recombinant cell of the disclosure include a monkey kidney CV1 cell transformed by SV40 (e.g., COS-7), a human embryonic kidney cell (e.g., HEK 293 or HEK 293 cell), a baby hamster kidney cell (BHK), a mouse sertoli cell (e.g., TM4 cells), a monkey kidney cell (CV1), a human cervical carcinoma cell (e.g., HeLa), canine kidney cell (e.g., MDCK), buffalo rat liver cell (e.g., BRL 3A), human lung cell (e.g., W138), human liver cell (e.g., Hep G2), mouse mammary tumor (e.g., MMT 060562), TRI cell, FS4 cell, a Chinese hamster ovary cell (CHO cell), an African green monkey kidney cell (e.g., Vero cell), a human A549 cell, a human cervix cell, a human CHME5 cell, a human PER.C6 cell, a NSO murine myeloma cell, a human epidermoid larynx cell, a human fibroblast cell, a human HUH-7 cell, a human MRC-5 cell, a human muscle cell, a human endothelial cell, a human astrocyte cell, a human macrophage cell, a human RAW 264.7 cell, a mouse 3T3 cell, a mouse L929 cell, a mouse connective tissue cell, a mouse muscle cell, and a rabbit kidney cell.

In some embodiments, the recombinant cell is selected from the group consisting of African green monkey kidney cell (Vero cell), baby hamster kidney (BHK) cell, Chinese hamster ovary cell (CHO cell), human A549 cell, human cervix cell, human CHME5 cell, human epidermoid larynx cell, human fibroblast cell, human HEK-293 cell, human HeLa cell, human HepG2 cell, human HUH-7 cell, human MRC-5 cell, human muscle cell, mouse 3T3 cell, mouse connective tissue cell, mouse muscle cell, and rabbit kidney cell. In some embodiments, the recombinant cell is a cell derived from a cell described above (i.e., a derivative cell of an original cell described herein) such as, for example, a cell that is either expanded from a clone of the original cell, an engineered version of the original cell, or a reclassification of the original cell after it has undergone extensive passaging, or has been passaged through another host.

In some embodiments of the disclosure, the recombinant cell is an insect cell, e.g., cell of an insect cell line. In some embodiments, the insect cell is a Sf21 cell. Additional suitable insect cell lines include, but are not limited to, cell lines established from insect orders Diptera, Lepidoptera and Hemiptera, and can be derived from different tissue sources. In some embodiments, the recombinant cell of the disclosure is a cell of a lepidopteran insect cell line. In the past few decades, the availability of lepidopteran insect cell lines has increased at about 50 lines per decade. More information regarding available lepidopteran insect cell lines can be found in, e.g., Lynn D. E., Available lepidopteran insect cell lines. Methods Mol. Biol. 2007; 388:117-38, which is herein incorporated by reference. In some embodiments, the recombinant cell is a mosquito cell, e.g., a cell of mosquito species within Anopheles (An.), Culex (Cx.) and Aedes (Stegomyia) (Ae.) genera. Exemplary mosquito cell lines suitable for the compositions and methods described herein include cell lines from the following mosquito species: Aedes aegypti, Aedes albopictus, Aedes pseudoscutellaris, Aedes triseriatus, Aedes vexans, Anopheles gambiae, Anopheles stephensi, Anopheles albimanus, Culex quinquefasciatus, Culex theileri, Culex tritaeniorhynchus, Culex bitaeniorhynchus, and Toxorhynchites amboinensis. Suitable mosquito cell lines include, but are not limited to, CCL-125, Aag-2, RML-12, C6/26, C6/36, C7-10, AP-61, A.t. GRIP-1, A.t. GRIP-2, UM-AVEl, Mos.55, SualB, 4a-3B, Mos.43, MSQ43, and LSB-AA695BB. In some embodiments, the mosquito cell is a cell of a C6/26 cell line.

### Cell Culture

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### Transgenic Animals

Also provided, in another aspect, are transgenic animals including a nucleic acid encoding a srRNA construct as described herein (e.g., vector or srRNA molecule). In some embodiments, the transgenic animal is a vertebrate animal or an invertebrate animal. In some embodiments, the transgenic animal is a mammal. In some embodiments, the transgenic mammal is a non-human mammal. Generally, transgenic animals of the present disclosure can be any non-human animal known in the art. In some embodiments, the non-human animals of the disclosure are non-human primates. Other animal species suitable for the compositions and methods of the disclosure include animals that are (i) suitable for transgenesis and (ii) capable of rearranging immunoglobulin gene segments to produce an antibody response. Examples of such species include but are not limited to mice, rats, hamsters, rabbits, chickens, goats, pigs, sheep and cows. Additional examples of non-human animals suitable for the compositions and methods of the disclosure can include, without limitation, laboratory animals (e.g., mice, rats, hamsters, gerbils, guinea pigs, etc.), livestock (e.g., horses, cattle, pigs, sheep, goats, ducks, geese, chickens, etc.), domesticated animals and pets (e.g. cats, dogs, etc.), non-human primates (e.g., apes, chimpanzees, orangutans, monkeys, etc.), fish, amphibians (e.g., frogs, salamanders, etc.), reptiles (e.g., snakes, lizards, etc.), and other animals (e.g., foxes, weasels, rabbits, mink, beavers, ermines, otters, sable, seals, coyotes, chinchillas, deer, muskrats, possums, etc.).

In some embodiments, the transgenic animal is an insect. In some embodiments, the insect is a mosquito. In some embodiments, the transgenic animals of the present disclosure are chimeric transgenic animals. In some embodiments, the transgenic animals of the present disclosure are transgenic animals with germ cells and somatic cells containing one or more (e.g., one or more, two or more, three or more, four or more, etc.) nucleic acid constructs of the present disclosure. In some embodiments, the one or more nucleic acid constructs are stably integrated into the genome of the transgenic animals. In some embodiments, the genomes of the transgenic animals of the present disclosure can comprise any of 1, 2, 3, 4, 5, 6, 7, 8, 9, 10, 11, 12, 13, 14, 15 or more copies of the one or more nucleic acid constructs of the present disclosure.

Approaches and methods for preparing transgenic non-human animals are known in the art. Exemplary methods include pronuclear microinjection, DNA microinjection, lentiviral vector mediated DNA transfer into early embryos and sperm-mediated transgenesis, adenovirus mediated introduction of DNA into animal sperm (e.g., in pig), retroviral vectors (e.g., avian species), somatic cell nuclear transfer (e.g., in goats). The state of the art in the preparation of transgenic domestic farm animals is reviewed in Niemann, H. et al. (2005) Rev. Sci. Tech. 24:285-298. In some embodiments, the transgenic non-human host animals of the disclosure are prepared using standard methods known in the art for introducing exogenous nucleic acid into the genome of a non-human animal. In some embodiments, the transgenic animals of the disclosure can be generated using classical random genomic recombination techniques or with more precise techniques such as guide RNA-directed CRISPR/Cas genome editing, or DNA-guided endonuclease genome editing with NgAgo (Natronobacterium gregoryi Argonaute), or TALENs genome editing (transcription activator-like effector nucleases). In some embodiments, the transgenic animals of the disclosure can be made using transgenic microinjection technology and do not require the use of homologous recombination technology and thus are considered to be easier to prepare and select than approaches using homologous recombination. In some embodiments, the transgenic animal produces a protein of interest as described herein.

### Pharmaceutical Compositions

The srRNA constructs, nucleic acid constructs, and recombinant cells of the disclosure can be incorporated into compositions, including pharmaceutical compositions. Such compositions generally include one or more of the srRNA constructs, nucleic acid constructs, and recombinant cells described and provided herein, and a pharmaceutically acceptable excipient, e.g., carrier. Accordingly, in one aspect of the present disclosure, provided herein are pharmaceutical compositions comprising a therapeutically acceptable excipient and one or more of the following: (1) a srRNA construct as disclosed herein, (2) a nucleic acid as disclosed herein, (3) a recombinant cell as disclosed herein. In some embodiments, provided herein are compositions including a srRNA construct as disclosed herein and a pharmaceutically acceptable excipient. In some embodiments, provided herein are compositions including a nucleic acid construct as disclosed herein and a pharmaceutically acceptable excipient. In some embodiments, provided herein are compositions including a recombinant cell as disclosed herein and a pharmaceutically acceptable excipient.

Non-limiting exemplary embodiments of the pharmaceutical compositions as described herein can include one or more of the following features. The srRNA constructs of the disclosure can be used in a naked form or formulated with a delivery vehicle. Exemplary routes, either using in a free form, e.g., inserted into a nucleic acid, e.g., a vector. For example, as described in greater detail below, a srRNA construct as described herein can be used as a vaccine.

In some embodiments, the compositions of the disclosure are formulated for the prevention, treatment, or management of a health condition such as an immune disease or a microbial infection. For example, the compositions of the disclosure can be formulated as a prophylactic composition, a therapeutic composition, or a pharmaceutical composition comprising a pharmaceutically acceptable excipient, or a mixture thereof.

In some embodiments, the compositions of the present disclosure are formulated for use as a vaccine. In some embodiments, the compositions of the present application are formulated for use as an adjuvant. In some embodiments, the compositions of the present disclosure are formulated for use as an immunogenic composition. In some embodiments, the compositions of the present application are formulated for use as a biotherapeutic.

For use in a pharmaceutical composition of the disclosure, a srRNA construct, a nucleic acid, or a recombinant cell as described herein can be formulated into or with delivery vehicles. Exemplary delivery vehicles suitable for the compositions and methods of the disclosure include, but are not limited to liposomes (e.g., neutral or anionic liposomes), microspheres, immune stimulating complexes (ISCOMS), lipid-based nanoparticles (LNP), polymer nanoparticles, viral replicon particles (VRPs), or conjugated with bioactive ligands, which can facilitate delivery and/or enhance the immune response. These compounds are readily available to one skilled in the art; for example, see Liposomes: A Practical Approach, RCP New Ed, TRL press (1990). Adjuvants other than liposomes and the like are also used and are known in the art. Adjuvants may protect the antigen (e.g., srRNA construct) from rapid dispersal by sequestering it in a local deposit, or they may contain substances that stimulate the host to secrete factors that are chemotactic for macrophages and other components of the immune system. An appropriate selection can be made by those skilled in the art, for example, from those described below.

Accordingly, in some embodiments, a composition of the disclosure can include one or more of the following: physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof.

In some embodiments, the srRNA constructs and nucleic acid constructs of the disclosure can be delivered to a cell or a subject by a lipid-based nanoparticle (LNP). LNP are generally less immunogenic than viral particles. While many humans have preexisting immunity to viral particles there is no pre-existing immunity to LNP. In addition, adaptive immune response against LNP is unlikely to occur which enables repeat dosing of LNP.

The lipids suitable for the compositions and methods described herein can be cationic lipids, ionizable cationic lipids, anionic lipids, or neutral lipids.

In some embodiments, the LNP of the disclosure can include one or more ionizable lipids. As used herein, the term “ionizable lipid” refers to a lipid that is cationic or becomes ionizable (protonated) as the pH is lowered below the pKa of the ionizable group of the lipid, but is more neutral at higher pH values. At pH values below the pKa, the lipid is then able to associate with negatively charged nucleic acids (e.g., oligonucleotides). As used herein, the term “ionizable lipid” includes lipids that assume a positive charge on pH decrease from physiological pH, and any of a number of lipid species that carry a net positive charge at a selective pH, such as physiological pH. Permanently cationic lipids such as DOTMA have proven too toxic for clinical use. The ionizable lipid can be present in lipid formulations according to other embodiments, preferably in a ratio of about 30 to about 70 Mol %, in some embodiments, about 30 Mol %, in other embodiments, about 40 Mol %, in other embodiments, about 45 Mol % in other embodiments, about 47.5 Mol % in other embodiments, about 50 Mol %, in still other embodiments, and about 60 Mol % in yet others (“Mol %” means the percentage of the total moles that is of a particular component). The term “about” in this paragraph signifies a plus or minus range of 5 Mol %. DODMA, or 1,2-dioleyloxy-3-dimethylaminopropane, is an ionizable lipid, as is DLin-MC3-DMA or 0-(Z,Z,Z,Z-heptatriaconta-6,9,26,29-tetraen-19-yl)-4-(N,N-dimethylamino) (“MC3”).

Exemplary ionizable lipids suitable for the compositions and methods of the disclosure includes those described in PCT publications WO2020252589A1 and WO2021000041A1, U.S. Pat. Nos. 8,450,298 and 10,844,028, and Love K. T. et al., Proc Natl Acad Sci USA, Feb. 2, 2010 107 (5) 1864-1869, all of which are hereby incorporated by reference in their entirety. Accordingly, in some embodiments, the LNP of the disclosure includes one or more lipid compounds described in Love K. T. et al. (2010 supra), such as C16-96, C14-110, and C12-200. In some embodiments, the LNP includes an ionizable cationic lipid selected from the group consisting of ALC-0315, C12-200, LN16, MC3, MD1, SM-102, and a combination of any thereof. In some embodiments, the LNP of the disclosure includes C12-200 lipid. The structure of C12-200 lipid is known in the art and described in, e.g., U.S. Pat. Nos. 8,450,298 and 10,844,028, which are hereby incorporated by reference in their entirety. In some embodiments the C12-200 is combined with cholesterol, C14-PEG2000, and DOPE. In some embodiments, the C12-200 is combined with DSPC and DMG-PEG2000.

In some embodiments, the LNP of the disclosure includes one or more cationic lipids. Several different ionizable cationic lipids have been developed for use in LNP. Suitable cationic lipids include, but are not limited to, 98N12-5, C12-200, C14-PEG2000, DLin-KC2-DMA (KC2), DLin-MC3-DMA (MC3), XTC, MD1, and 7C1. In one type of LNP, a GalNAc moiety is attached to the outside of the LNP and acts as a ligand for uptake in to the liver via the asialoglycoprotein receptor. Any of these cationic lipids can be used to formulate LNP for delivery of the srRNA constructs and nucleic acid constructs of the disclosure.

In some embodiments, the LNP of the disclosure includes one or more neutral lipids. Non-limiting neutral lipids suitable for the compositions and methods of the disclosure include DPSC, DPPC, POPC, DOPE, and SM. In some embodiments, the LNP of the disclosure includes one or more ionizable lipid compounds described in PCT publications WO2020252589A1 and WO2021000041A1.

A number of other lipids or combination of lipids that are known in the art can be used to produce a LNP. Non-limiting examples of lipids suitable for use to produce LNPs include DOTMA, DOSPA, DOTAP, DMRIE, DC-cholesterol, DOTAP-cholesterol, GAP-DMORIE-DPyPE, and GL67A-DOPE-DMPE-polyethylene glycol (PEG). Additional non-limiting examples of cationic lipids include 98N12-5, C12-200, C14-PEG2000, DLin-KC2-DMA (KC2), DLin-MC3-DMA (MC3), XTC, MD1, 7C1, and a combination of any thereof. Additional non-limiting examples of neutral lipids include DPSC, DPPC, POPC, DOPE, and SM. Non-limiting examples of PEG-modified lipids include PEG-DMG, PEG-CerC14, and PEG-CerC20.

In some embodiments, the mass ratio of lipid to nucleic acid in the LNP delivery system is about 100:1 to about 3:1, about 70:1 to 10:1, or 16:1 to 4:1. In some embodiments, the mass ratio of lipid to nucleic acid in the LNP delivery system is about 16:1 to 4:1. In some embodiments, the mass ratio of lipid to nucleic acid in the LNP delivery system is about 20:1. In some embodiments, the mass ratio of lipid to nucleic acid in the LNP delivery system is about 8:1. In some embodiments, the lipid-based nanoparticles have an average diameter of less than about 1000 nm, about 500 nm, about 250 nm, about 200 nm, about 150 nm, about 100 nm, about 75 nm, about 50 nm, or about 25 nm. In some embodiments, the LNPs have an average diameter ranging from about 70 nm to 100 nm. In some embodiments, the LNPs have an average diameter ranging from about 88 nm to about 92 nm, from 82 nm to about 86 nm, or from about 80 nm to about 95 nm.

In some embodiments, the compositions of the disclosure are formulated in a polymer nanoparticle. In some embodiments, the compositions are immunogenic compositions, e.g., composition that can stimulate an immune response in a subject. In some embodiments, the immunogenic compositions are formulated as a vaccine. In some embodiments, the pharmaceutical compositions are formulated as an adjuvant.

In some embodiments, the immunogenic compositions are substantially non-immunogenic to a subject, e.g., compositions that minimally stimulate an immune response in a subject. In some embodiments, the non-immunogenic or minimally immunogenic compositions are formulated as a biotherapeutic. In some embodiments, the pharmaceutical compositions are formulated for one or more of intranasal administration, transdermal administration, intraperitoneal administration, intramuscular administration, intratracheal administration, intranodal administration, intratumoral administration, intraarticular administration, intravenous administration, subcutaneous administration, intravaginal administration, intraocular, rectal, and oral administration.

Pharmaceutical compositions suitable for injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In these cases, the composition should be sterile and should be fluid to the extent that easy syringeability exists. It can be stable under the conditions of manufacture and storage, and can be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above.

In some embodiments, the pharmaceutical compositions of the disclosure are formulated for inhalation, such as an aerosol, spray, mist, liquid, or powder. Administration by inhalation may be in the form of either dry powders or aerosol formulations, which are inhaled by a subject (e.g., a patient) either through use of an inhalation device, e.g., a microspray, a pressurized metered dose inhaler, or nebulizer.

In some embodiments, the composition is formulated for one or more of intranasal administration, transdermal administration, intramuscular administration, intranodal administration, intratumoral administration, intraarticular administration, intravenous administration, intraperitoneal administration, intratracheal administration, oral administration, intravaginal, intraocular, rectal, or intra-cranial administration. In some embodiments, the administered composition results in an increased production of interferon in the subject.

### Kits

Also provided herein are various kits for the practice of a method described herein. In particular, some embodiments of the disclosure provide kits for eliciting an immune response in a subject. Some other embodiments relate to kits for the prevention of a health condition in a subject in need thereof. Some other embodiments relate to kits for methods of treating a health condition in a subject in need thereof. For example, provided herein, in some embodiments, are kits that include one or more of the srRNA constructs, nucleic acid constructs, recombinant cells, and/or pharmaceutical compositions as provided and described herein, as well as written instructions for making and using the same.

In some embodiments, the kits of the disclosure further include one or more means useful for the administration of any one of the provided srRNA constructs, nucleic acid constructs, recombinant cells, and/or pharmaceutical compositions to a subject. For example, in some embodiments, the kits of the disclosure further include one or more syringes (including pre-filled syringes) and/or catheters (including pre-filled syringes) used to administer any one of the provided srRNA constructs, nucleic acid constructs, recombinant cells, and/or pharmaceutical compositions to a subject. In some embodiments, a kit can have one or more additional therapeutic agents that can be administered simultaneously or sequentially with the other kit components for a desired purpose, e.g., for eliciting an immune response, for preventing or treating a condition in a subject in need thereof.

Any of the above-described kits can further include one or more additional reagents, where such additional reagents can be selected from: dilution buffers, reconstitution solutions, wash buffers, control reagents, control expression vectors, negative controls, positive controls, reagents suitable for in vitro production of the provided srRNA constructs, nucleic acid constructs, recombinant cells, and/or pharmaceutical compositions of the disclosure.

In some embodiments, the components of a kit can be in separate containers. In some other embodiments, the components of a kit can be combined in a single container. Accordingly, in some embodiments of the disclosure, the kit includes one or more of the nucleic acid constructs (e.g., vectors or srRNA molecules), recombinant cells, recombinant polypeptides, and/or pharmaceutical compositions as provided and described herein in one container (e.g., in a sterile glass or plastic vial) and a further therapeutic agent in another container (e.g., in a sterile glass or plastic vial).

In another embodiment, the kit includes a combination of the compositions described herein, including one or more nucleic acid constructs (e.g., vectors or srRNA molecules), recombinant cells, and/or recombinant polypeptides of the disclosure in combination with one or more further therapeutic agents formulated together in a pharmaceutical composition and, optionally, in a single, common container.

If the kit includes a pharmaceutical composition for parenteral administration to a subject, the kit can include a device (e.g., an injection device or catheter) for performing such administration. For example, the kit can include one or more hypodermic needles or other injection devices as discussed above containing one or more nucleic acid constructs (e.g., vectors or srRNA molecules), recombinant cells, and/or recombinant polypeptides of the disclosure.

In some embodiments, a kit can further include instructions for using the components of the kit to practice the methods disclosed herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

### Methods for Inducing an Immune Response, Preventing, or Treating Health Conditions

Administration of any one of the compositions described herein, e.g., srRNA constructs, nucleic acid constructs, recombinant cells, and/or pharmaceutical compositions, can be used in the treatment of relevant health conditions, such as proliferative disorders (e.g., cancers), infectious diseases (e.g., acute infections, chronic infections, or viral infections), rare diseases, and/or autoimmune diseases, and/or inflammatory diseases. In some embodiments, the srRNA constructs, nucleic acid constructs, recombinant cells, and/or pharmaceutical compositions as described herein can be useful for inducing a pharmacodynamic effect in a subject. In some embodiments, the srRNA constructs, nucleic acid constructs, recombinant cells, and/or pharmaceutical compositions as described herein can be useful for modulating, e.g., eliciting or suppressing, an immune response in a subject in need thereof. In some embodiments, the srRNA constructs, nucleic acid constructs, recombinant cells, and/or pharmaceutical compositions as described herein can be incorporated into therapeutic agents for use in methods of treating a subject who has, who is suspected of having, or who may be at high risk for developing one or more relevant health conditions or diseases. Exemplary health conditions or diseases can include, without limitation, cancers, immune diseases, autoimmune diseases, inflammatory diseases, gene therapy, gene replacement, cardiovascular diseases, age-related pathologies, rare disease, acute infection, and chronic infection. In some embodiments, the subject is a patient under the care of a physician.

Examples of autoimmune diseases suitable for the methods of the disclosure include, but are not limited to, rheumatoid arthritis, osteoarthritis, Still's disease, Familiar Mediterranean Fever, systemic sclerosis, multiple sclerosis, ankylosing spondylitis, Hashimoto's tyroidism, systemic lupus erythematosus, Sjogren's syndrome, diabetic retinopathy, diabetic vasculopathy, diabetic neuralgia, insulitis, psoriasis, alopecia greata, warm and cold autoimmune hemolytic anemia (AIHA), pernicious anemia, acute inflammatory diseases, autoimmune adrenalitis, chronic inflammatory demyelinating polyneuropathy (CIDP), Lambert-Eaton syndrome, lichen sclerosis, Lyme disease, Graves disease, Behget's disease, Meniere's disease, reactive arthritis (Reiter's syndrome), Churg-Strauss syndrome, Cogan syndrome, CREST syndrome, Pemphigus vulgaris and Pemphigus foliaceus, bullous pemphigoid, polymyalgia rheumatica, polymyositis, primary biliary cirrhosis, pancreatitis, peritonitis, psoriatic arthritis, rheumatic fever, sarcoidosis, Sjörgensen syndrome, scleroderma, celiac disease, stiff-man syndrome, Takayasu arteritis, transient gluten intolerance, autoimmune uveitis, vitiligo, polychondritis, dermatitis herpetiformis (DH) or Duhring's disease, fibromyalgia, Goodpasture syndrome, Guillain-Barré syndrome, Hashimoto thyroiditis, autoimmune hepatitis, inflammatory bowel disease (IBD), Crohn's disease, colitis ulcerosa, myasthenia gravis, immune complex disorders, glomerulonephritis, polyarteritis nodosa, anti-phospholipid syndrome, polyglandular autoimmune syndrome, idiopatic pulmonar fibrosis, idiopathic thrombocytopenic purpura (ITP), urticaria, autoimmune infertility, juvenile rheumatoid arthritis, sarcoidosis, and autoimmune cardiomyopathy.

Non-limiting examples of infection suitable for the methods of the disclosure include infections with viruses such as human immunodeficiency virus (HIV), hepatitis B virus (HBV), hepatitis B virus (HCV), Cytomegalovirus (CMV), respiratory syncytial virus (RSV), human papillomavirus (HPV), Epstein-Barr virus (EBV), severe acute respiratory syndrome coronavirus 2 (SARS-CoV2), severe acute respiratory syndrome coronavirus (SARS-CoV), Middle East Respiratory Syndrome (MERS), influenza virus, and Ebola virus. Additional infections suitable for the methods of the disclosure include infections with intracellular parasites such as Leishmania, Rickettsia, Chlamydia, Coxiella, Plasmodium, Brucella, mycobacteria, Listeria, Toxoplasma and Trypanosoma. In some embodiments, the srRNA constructs, nucleic acid constructs, recombinant cells, and/or pharmaceutical compositions, can be useful in the treatment and/or prevention of immune diseases, autoimmune diseases, or inflammatory diseases such as, for example, glomerulonephritis, inflammatory bowel disease, nephritis, peritonitis, psoriatic arthritis, osteoarthritis, Still's disease, Familiar Mediterranean Fever, systemic scleroderma and sclerosis, inflammatory bowel disease (IBD), Crohn's disease, ulcerative colitis, acute lung injury, meningitis, encephalitis, uveitis, multiple myeloma, glomerulonephritis, nephritis, asthma, atherosclerosis, leukocyte adhesion deficiency, multiple sclerosis, Raynaud's syndrome, Sjögren's syndrome, juvenile onset diabetes, Reiter's disease, Behcet's disease, immune complex nephritis, IgA nephropathy, IgM polyneuropathies, immune-mediated thrombocytopenias, hemolytic anemia, myasthenia gravis, lupus nephritis, lupus erythematosus, rheumatoid arthritis (RA), ankylosing spondylitis, pemphigus, Graves' disease, Hashimoto's thyroiditis, small vessel vasculitides, Omen's syndrome, chronic renal failure, autoimmune thyroid disease, acute infectious mononucleosis, HIV, herpes virus associated diseases, human virus infections, coronavirus, other enterovirus, herpes virus, influenza virus, parainfluenza virus, respiratory syncytial virus or adenovirus infection, bacteria pneumonia, wounds, sepsis, cerebral stroke/cerebral edema, ischaemia-reperfusion injury, and hepatitis C.

Non-limiting examples of inflammatory suitable for the methods of the disclosure include inflammatory diseases such as asthma, inflammatory bowel disease (IBD), chronic colitis, splenomegaly, and rheumatoid arthritis.

In one aspect, provided herein are methods for inducing a pharmacodynamic effect in a subject, the methods include administering to the subject a composition including one or more of the following: (a) replicon, e.g., self-replicating RNA (srRNA) construct as described herein; (b) a nucleic acid as described herein; (c) a recombinant cell as described herein; and (d) a pharmaceutical composition as described herein. In some embodiments, the pharmacodynamic effect includes eliciting an immune response in the subject.

In another aspect, provided herein are methods for preventing or treating a health condition in a subject, the methods include prophylactically or therapeutically administering to the subject a composition including one or more of the following: (a) a replicon, e.g., self-replicating RNA (srRNA) construct as described herein; (b) a nucleic acid as described herein; (c) a recombinant cell as described herein; and (d) a pharmaceutical composition as described herein.

Non-limiting exemplary embodiments of the methods for inducing a pharmacodynamic effect, and/or preventing, and/or treating a health condition in a subject as described herein can include one or more of the following features. In some embodiments, the administered composition induces production of one or more pro-inflammatory molecules in the subject. In some embodiments, the one or more pro-inflammatory molecules includes interferon gamma (IFNγ), cytokines, TNF-α, GM-CSF, and MIP1α, granzyme B, granzyme A, perforin, or a combination of any thereof. In some embodiments, the subject has been previously treated with one or more therapies and has developed at least a partial resistance to said one or more therapies. In some embodiments, at least one of the one or more therapies includes a small molecule. In some embodiments, the health condition is a proliferative disorder, inflammatory disorder, autoimmune disorder, or a microbial infection. In some embodiments, the subject has or is suspected of having a health condition associated with proliferative disorder, inflammatory disorder, autoimmune disorder, or a microbial infection. In some embodiments, the proliferative disorder is a cancer. In some embodiments, the cancer is a breast cancer.

Non-limiting examples of breast cancer suitable for the methods of the disclosure include breast ductal cancer, breast lobular cancer, breast undifferentiated cancer, breast lobular sarcoma, breast angiosarcoma, and primary breast lymphoma. Breast cancer may include stage I, stage II, stage IIIA, stage IIIB, stage IIIC and stage IV breast cancer. Breast ductal carcinomas can include invasive carcinoma types, invasive carcinoma in situ with predominant intraglandular components, inflammatory breast cancers, and ductal carcinomas of the breast. Breast ductal carcinomas can include invasive lobular carcinomas with predominant in situ components, invasive lobular carcinomas, and infiltrating lobular carcinomas. Breast cancer may include Paget's disease, extramammary Paget's disease, Paget's disease with intraglandular cancer, and Paget's disease with invasive ductal carcinoma. Breast cancer may include breast neoplasms with histological and hyperstructural heterogeneity (e.g., mixed cell types). Breast cancer can be classified as basal-like, luminal A, luminal B, ERBB2/Her2+ or normal breast-like molecular subtypes.

In some embodiments, the disclosed compositions can be formulated to be compatible with its intended route of administration. For example, the srRNA constructs, nucleic acid constructs, recombinant cells, and/or pharmaceutical compositions of the disclosure may be given orally, by inhalation, or through a parenteral route. Examples of parenteral routes of administration include, for example, intramuscular, intratumoral, intraocular, intravenous, intranodal, intradermal, subcutaneous, transdermal (topical), transmucosal, intravaginal, and rectal administration. In some embodiments, the composition is administered intramuscularly. In some embodiments, the composition is administered intratumorally. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates, phosphates, tris, sucrose and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

The compositions described herein, e.g., srRNA constructs, nucleic acid constructs, recombinant cells, and/or pharmaceutical compositions, can be administered one from one or more times per day to one or more times per week; including once every other day. Treatment of a subject with a therapeutically effective amount of the subject srRNA constructs, nucleic acid constructs, recombinant cells, and/or pharmaceutical compositions of the disclosure can include a single treatment or, can include a series of treatments. In some embodiments, the compositions are administered at weekly intervals, e.g., 1 to 2, 2 to 3, or 3 to 4 doses given at 1 to 2, 2 to 3, or 3 to 4 week intervals. This may be followed with an additional administration every 1, 2, 3, or 4 months. In some embodiments, 3 doses can be administered intramuscularly at a 3 to 4 week interval, followed by intramuscular administration every 3 months. Alternatively, the composition can be administered at shorter intervals, e.g., every 8 hours for five days, followed by a rest period of 2 to 14 days, e.g., 9 days, followed by an additional five days of administration every 8 hours. With regard to srRNA constructs and nucleic acid constructs, the therapeutically effective amount of a srRNA construct and nucleic acid construct of the disclosure (e.g., an effective dosage) depends on the srRNA construct and nucleic acid construct selected.

As discussed supra, a therapeutically effective amount includes an amount of a therapeutic composition that is sufficient to promote a particular effect when administered to a subject, such as one who has, is suspected of having, or is at risk for a health condition, e.g., a cancer. In some embodiments, an effective amount includes an amount sufficient to prevent or delay the development of a symptom of the disease, alter the course of a symptom of the disease (for example but not limited to, slow the progression of a symptom of the disease), or reverse a symptom of the disease.

A treatment is considered effective treatment if at least any one or all of the signs or symptoms of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art and/or described herein. Treatment includes any treatment of a disease in a subject or an animal (some non-limiting examples include a human, or a mammal) and includes: (1) inhibiting the disease, e.g., arresting, or slowing the progression of symptoms; or (2) relieving the disease, e.g., causing regression of symptoms; and (3) preventing or reducing the likelihood of the development of symptoms.

In some embodiments, the srRNA constructs, nucleic acid constructs, recombinant cells, and/or pharmaceutical compositions of the disclosure can be administered to a subject in a composition having a pharmaceutically acceptable carrier and in an amount effective to stimulate an immune response. Generally, a subject can be immunized through an initial series of injections (or administration through one of the other routes described below) and subsequently given boosters to increase the protection afforded by the original series of administrations. The initial series of injections and the subsequent boosters are administered in such doses and over such a period of time as is necessary to stimulate an immune response in a subject. In some embodiments of the disclosed methods, the subject is a mammal. In some embodiments, the mammal is a human subject.

As described above, pharmaceutically acceptable carriers suitable for injectable use include sterile aqueous solutions (where water soluble) or dispersions and sterile powders for the extemporaneous preparation of sterile injectable solutions or dispersions. In these cases, the composition must be sterile and must be fluid to the extent that easy syringeability exists. The composition must further be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, etc.), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like.

Sterile injectable solutions can be prepared by incorporating the srRNA constructs, nucleic acid constructs, recombinant cells, and/or pharmaceutical compositions in the required mount in an appropriate solvent with one or a combination of ingredients enumerated above, as required, followed by filtered sterilization.

When the srRNA constructs, nucleic acid constructs, recombinant cells, and/or pharmaceutical compositions are suitably protected, as described above, they may be orally administered, for example, with an inert diluent or an assimilable edible carrier. The srRNA constructs, nucleic acid constructs, recombinant cells, and/or pharmaceutical compositions and other ingredients may also be enclosed in a hard or soft shell gelatin capsule, compressed into tablets, or incorporated directly into the individual's diet. For oral therapeutic administration, the active compound may be incorporated with excipients and used in the form of ingestible tablets, buccal tablets, troches, capsules, elixirs, suspensions, syrups, wafers, and the like.

### Additional Therapies

In some embodiments, a composition according to the present disclosure is administered to the subject individually as a single therapy (monotherapy) or as a first therapy in combination with at least one additional therapies (e.g., second therapy). In some embodiments, the second therapy is selected from the group consisting of chemotherapy, radiotherapy, immunotherapy, hormonal therapy, toxin therapy, targeted therapy, and surgery. In some embodiments, the second therapy is selected from the group consisting of chemotherapy, radiotherapy, immunotherapy, hormonal therapy, toxin therapy or surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B. V., the disclosures of which are incorporated herein by reference.

Additional embodiments are disclosed in further detail in the following examples, which are provided by way of illustration and are not in any way intended to limit the scope of this disclosure or the claims.

### Example 1

Construction of EEEV Vectors Encoding srRNA Constructs for Vaccine Applications

This Example describes the experiments performed to construct a base EEEV vector (e.g., without a heterologous gene) that was subsequently used for construction of a EEEV vectors that express a gene or genes of interest (e.g., ESR1 or variants thereof, PI3K or variants thereof, HER2 or a variant thereof, and HER3 or a variant thereof).

The base EEEV vector (i.e. without a heterologous gene of interest) was constructed as follows: The base EEEV vector was synthesized de novo in four ˜4 kb parts (Twist Bioscience) from a reference sequence (Genbank EF151502) with several modifications. Silent mutations G301A, A3550C, G4516A, G5725A, and G7399A were incorporated to eliminate restriction enzyme cut sites. A unique restriction enzyme cut site (SpeI, 5‘-A’CTAG,T-3′) was incorporated in place of the coding sequence of the native EEEV structural genes (where the 5′ A matches the location of the structural polyprotein ATG start codon, and the 3′ T matches the location of the structural polyprotein stop codon TAA). A 5′ adaptor sequence (5′-CTGGAGACGTGGAGGAGAACCCTGGACCT-3′; SEQ ID NO: 5) was inserted upstream of the SpeI site, and a 3′ adaptor sequence (5′-GACCGCTACGCCCCAATGACCCGACCAGC-3′; SEQ ID NO: 6) was inserted downstream of the SpeI site for subsequent Gibson Assembly® procedures (Gibson et al., Nat. Methods 6, 343-345, 2009). A bacteriophage T7 RNA polymerase promoter (5′-TAATACGACTCACTATAG-3′; SEQ ID NO: 7) was included upstream of the EEEV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 8) followed by a unique restriction enzyme cut site (NotI, 5‘-GC’GGCC,GC-3′). The parts were combined in a five-piece Gibson Assembly® reaction: a linearized pYL backbone and the four synthesized fragments to result in the EEEV base vector.

Construction of an EEEV vector containing heterologous genes was carried out as follows: the base EEEV base vector was linearized by SpeI digestion. The ESR1, PI3K, HER2, and HER3 variants were codon optimized/refactored for human expression in silico and along with the EMCV IRES, were synthesized de novo (GeneArt, IDT). The synthetic products were amplified using primers which added either 5′ and 3′ adaptor sequences to the ends of the genes, or primers which added P2A sequences and/or sequences of homology to neighboring gene inserts. The digestion product and PCR products were combined by Gibson Assembly® procedure to result in the final vectors.

### Example 2

In Vitro Evaluation of EEEV Vectors Encoding Vaccine srRNA Constructs

This Example describes the results of in vitro experiments performed to evaluate expression levels of the synthetic EEEV srRNA constructs described in Example 1 above, and to investigate any differential behavior thereof (e.g., replication and protein expression).

In vitro transcription: RNA was prepared by in vitro transcription from a SapI-linearized plasmid template with bacteriophage T7 polymerase with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA was then purified using phenol/chloroform extraction, or column purification (Monarch® RNA Cleanup Kit, NEB). RNA concentration was determined by absorbance at 260 nm (Nanodrop, Thermo Fisher Scientific).

Replication: RNA was transformed by electroporation into BHK-21 or Vero cells (e.g., 4D-Nucleofector™, Lonza). At 15-22 hours following transformation, the cells were fixed and permeabilized (eBioscience™ Foxp3/Transcription Factor Staining Buffer Set, Invitrogen) and stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

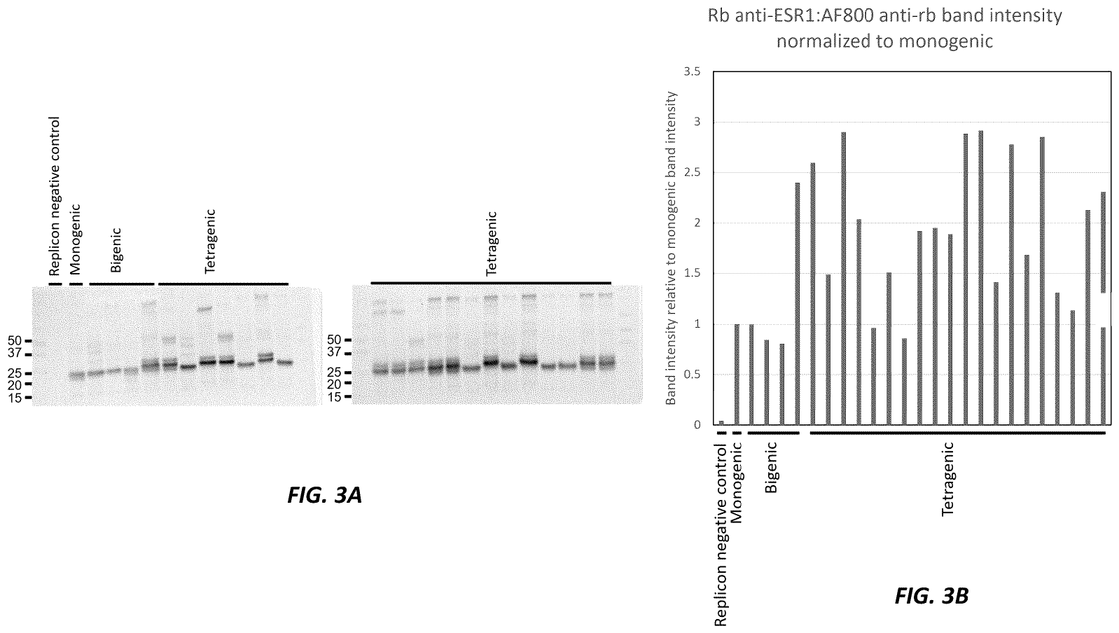

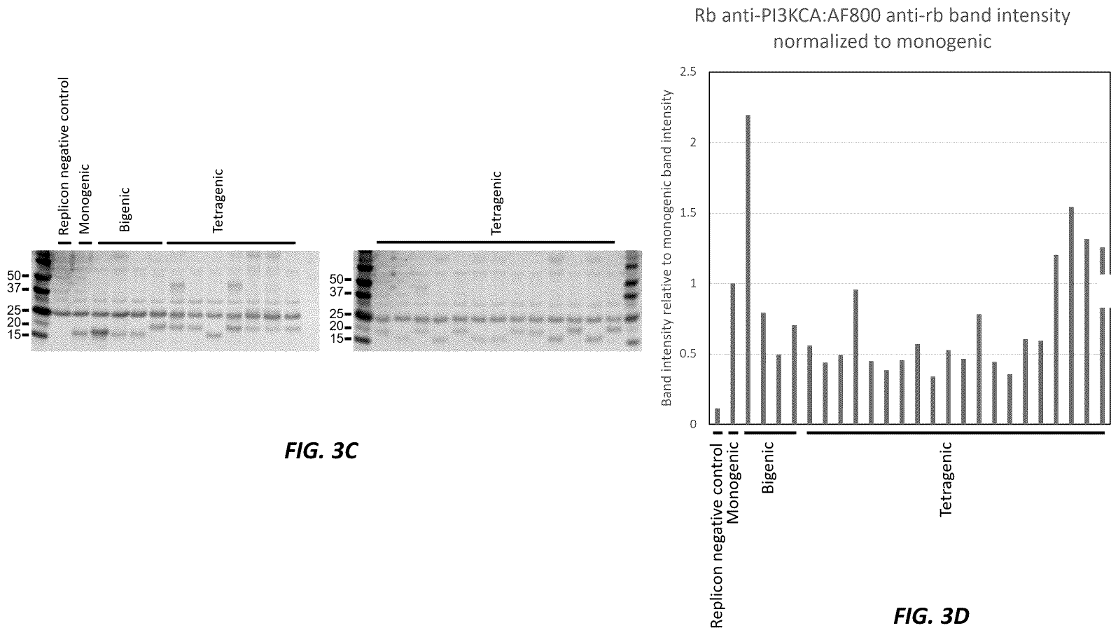

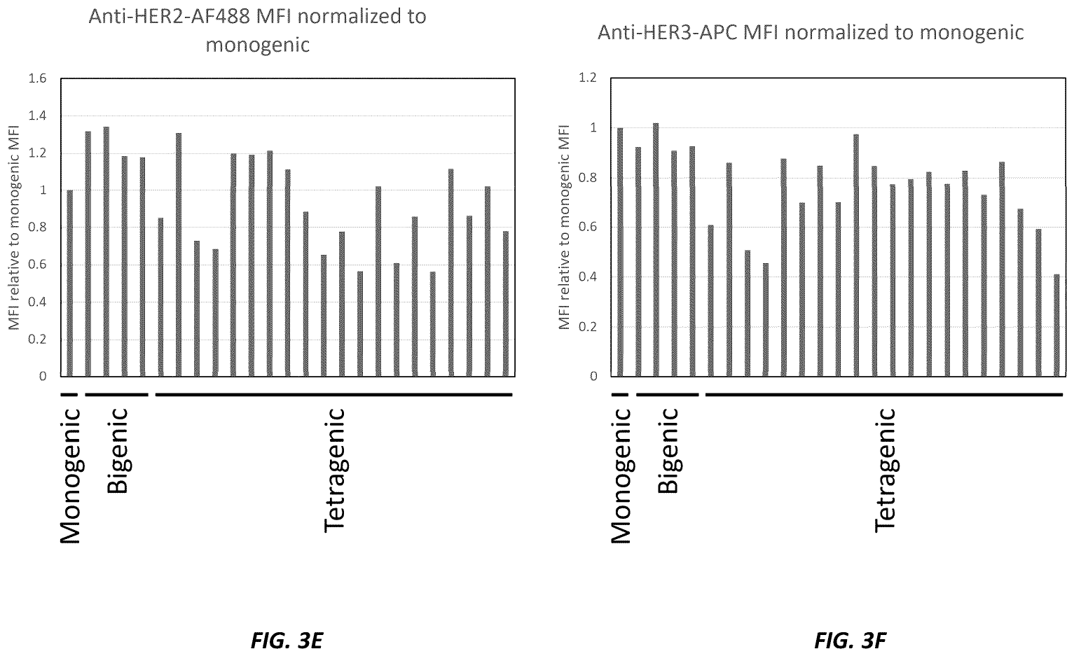

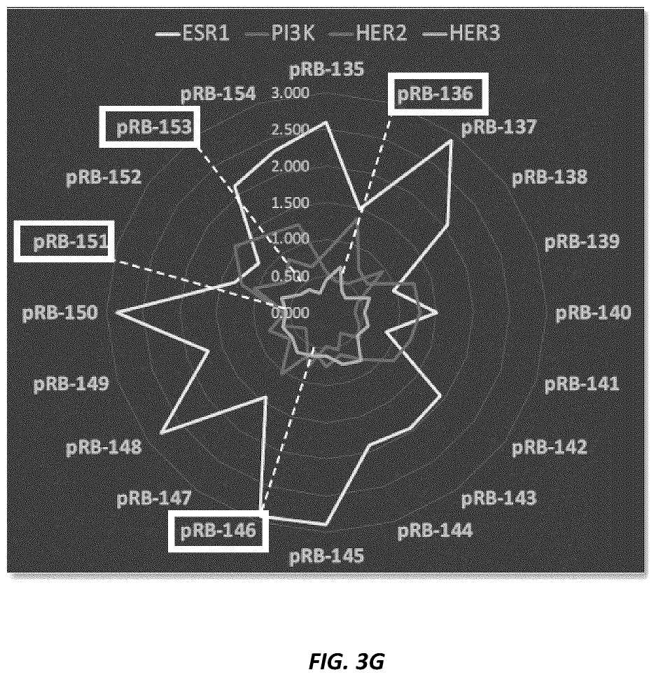

Protein expression: RNA was transformed by electroporation into BHK-21 or Vero cells (e.g., 4D-Nucleofector™, Lonza). ESR1: At 15-22 hours following transformation, the cells were collected and lysed in RIPA buffer. Lysate protein concentration was normalized, then probed in an immunoblot with an anti-ERα rabbit antibody (A300-497A, Bethyl) and imaged using an AF800 conjugated anti-rabbit goat antibody (A32735, Thermo) (FIG. 3A). The fluorescence signal from the cell samples transformed by a synthetic monogenic EEEV replicon expressing ESR1 was used to normalize expression levels to evaluate relative ESR1 expression from the panel of bigenic and tetragenic replicons (FIG. 3B). PI3K: At 15-22 hours following transformation, the cells were collected and lysed in RIPA buffer. Lysate protein concentration was normalized, then probed in an immunoblot with an anti-PI3KCA rabbit antibody (PA587398, Thermo) and imaged using an AF800 conjugated anti-rabbit goat antibody (A32735, Thermo) (FIG. 3C). The fluorescence signal from the cell samples transformed by a synthetic monogenic EEEV replicon expressing PI3K was used to normalize expression levels to evaluate relative PI3K expression from the panel of bigenic and tetragenic replicons (FIG. 3D). HER2: At 15-22 hours following transformation, the cells were fixed and permeabilized (eBioscience™ Foxp3/Transcription Factor Staining Buffer Set, Invitrogen) and stained using an AF488-conjugated anti-HER2 mouse monoclonal antibody (24D2, Biolegend). The mean fluorescence intensity (MFI) of AF488 was used as the readout of HER2 expression. The MFI of cells transformed by a synthetic monogenic EEEV replicon expressing HER2 was used to normalize expression levels to evaluate relative HER2 expression from the panel of bigenic and tetragenic replicons (FIG. 3E). HER3: At 15-22 hours following transformation, the cells were fixed and permeabilized (eBioscience™ Foxp3/Transcription Factor Staining Buffer Set, Invitrogen) and stained using an APC-conjugated anti-HER3 mouse monoclonal antibody (IB4C3, Biolegend). The mean fluorescence intensity (MFI) of APC was used as the readout of HER3 expression. The MFI of cells transformed by a synthetic monogenic EEEV replicon expressing HER3 was used to normalize expression levels to evaluate relative HER3 expression from the panel of bigenic and tetragenic replicons (FIG. 3F). The normalized ESR1, PI3K, HER2, and HER3 expression data from the tetragenic replicons was visualized on a spider graph (FIG. 3G).

### Example 3

In Vivo Evaluation of EEEV Vectors Encoding Vaccine srRNA Constructs

This Example describes the results of in vivo experiments performed to evaluate any differential immune responses following vaccination with the synthetic EEEV replicon constructs described herein (e.g., both unformulated and LNP formulated vectors).

In these experiments, synthetic replicon constructs derived from the EEEV strain FL93-939 were designed and subsequently evaluated.

Mice and injections. BALB/c mice were purchased from Charles River Labs, Envigo, or Jackson Laboratories. On day of dosing, between 0.01-10 μg of material was injected intramuscularly either into one or split into both quadricep muscles. Vectors were administered either unformulated in saline, or LNP-formulated. Animals were monitored for body weight and other general observations throughout the course of the study. For immunogenicity studies, animals were dosed on Day 0 only or Day 0 and Day 21.

LNP formulation. Replicon RNA was formulated in lipid nanoparticles using a microfluidics mixer and analyzed for particle size, polydispersity using dynamic light scattering and encapsulation efficiency. Lipids were suspended in ethanol. For C12-200, RNA was suspended in 10 mM citrate buffer pH 5.0 a concentration of 172 μg/ml, and was mixed at a flow rate of 3:1 (aqueous:organic). For L2, RNA was suspended in 250 mM NaOAc pH 4.0 at a concentration of 82 μg/ml, and was mixed at a flow rate of 3:1 (aqueous:organic).

ELISpot. To measure the magnitude of ESR1-, HER2, and HER3-specific T cell responses, IFNγ ELISpot analysis was performed using Mouse IFNγ ELISpot PLUS Kit (RP) (MabTech) as per manufacturer's instructions. In brief, splenocytes were isolated and resuspended to a concentration of 5×10⁶ cells/mL in media containing peptides derived from ESR1, HER2, and HER3, PMA/ionomycin as a positive control, or DMSO as a mock stimulation.

### Evaluation of Linkers

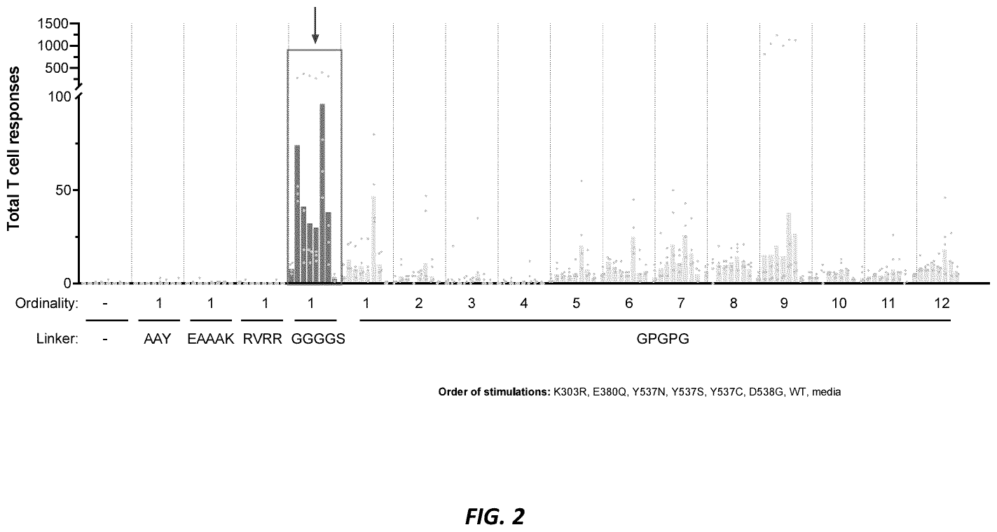

Results of mouse IFNγ detecting ELISpot assay as measured by spot-forming units corresponding to responder splenic T cells 14 days after intramuscular injection of monogenic replicon RNA encoding ESR1 mutations in different ordinalities and inter-connected by different linkers is shown in FIG. 2. AAY, EAAAK (SEQ ID NO: 1), RVRR (SEQ ID NO: 2), GGGGS (SEQ ID NO: 3), and GPGPG (SEQ ID NO: 4) linkers that were tested in varying ordinalities in an ESR1 antigen cassette containing the K303R, E380Q, Y537C, Y537S, Y537N, and D538G mutations. Columns for each cassette corresponds to the following stimulation conditions with a single peptide in the order of K303R, E380Q, Y537N, Y537S, Y537C, D538G, wild-type ESR1, and media. The total T cell responses (plotted as counted spot-forming units per million of cells) are shown the y-axis. The GGGGS (SEQ ID NO: 3) linker ordinality produced the most robust T cell responses.

### Evaluation of Number and Ordinality of Genes

Results of mouse IFNγ detecting ELISpot assay as measured by spot-forming units corresponding to responder splenic T cells at Day 35 after two intramuscular injections of replicon RNA encoding ESR1, HER2, and HER3 are shown in FIG. 4. Different constructs, in either monogenic, bigenic or tetragenic form, having different ordinalities and connecting sequences of ESR1, PI3K, HER2, and HER3, were tested in order to determine which configuration of genes in the constructs yielded the most robust T-cell responses upon stimulation. The Y-axis shows the total T cell responses. PI3K responses were not measured in this experiment because it does not form responses in BALB/c mice.

### Evaluation of Ordinality of Genes and Lipid Formulation

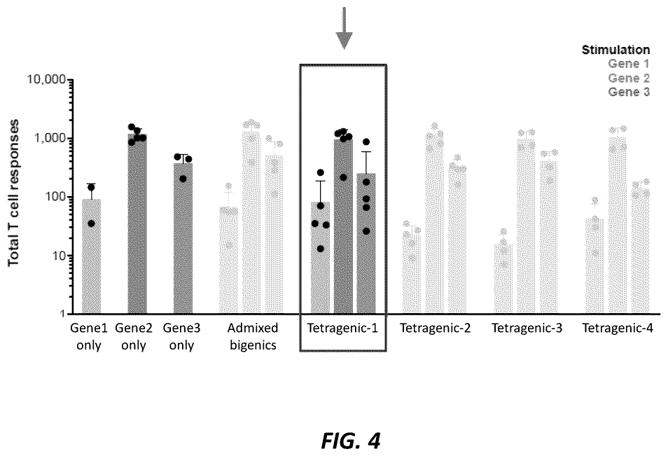

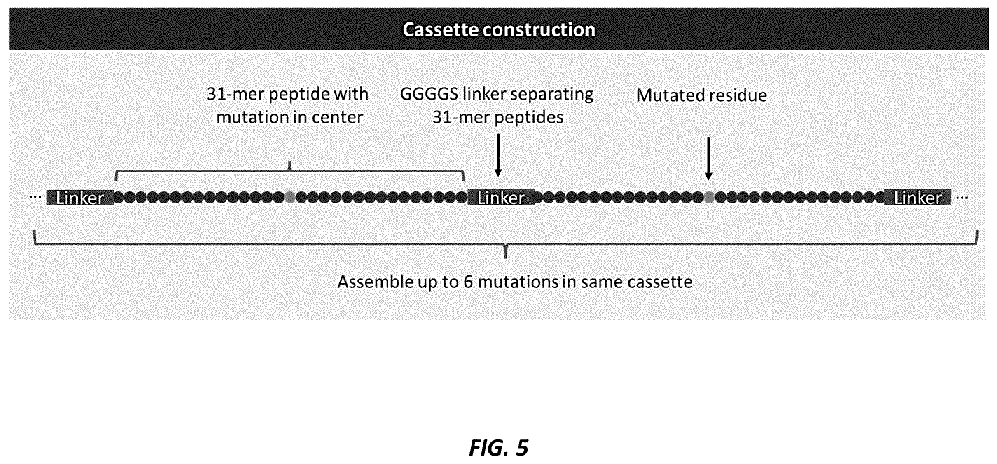

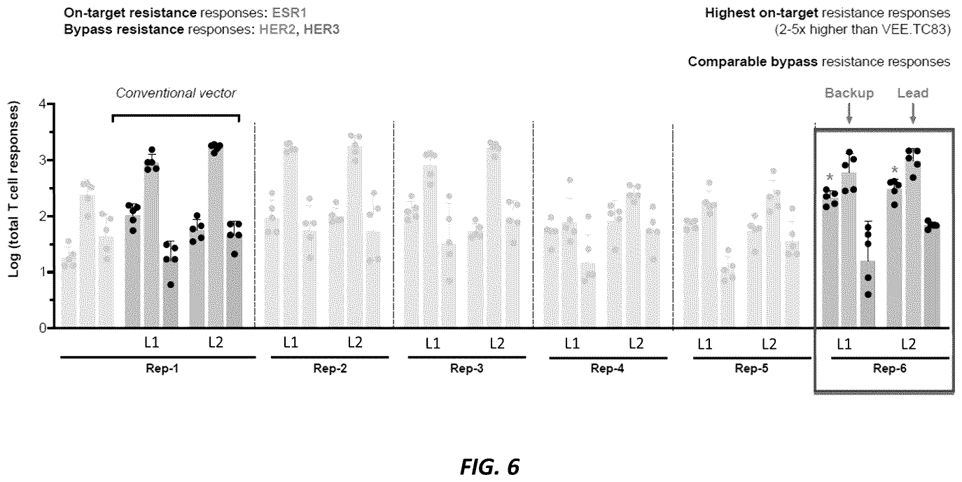

Results of mouse IFNγ detecting ELISpot assay as measured by spot-forming units corresponding to responder splenic T cells at Day 35 after two intramuscular injections of replicon RNA either in saline, or formulated in two different LNP compositions L1 or L2 encoding ESR1, PI3K, HER2, and HER3 are shown in FIG. 6. Different tetragenic constructs, having different replicon vector backbones were tested to determine which RNA replicon vector and formulation yielded the most robust T cell responses upon stimulation.

### Example 4

### Estrogen Receptor Positive Breast Cancer Efficacy

Two efficacy models are shown in FIG. 7 to mimic two clinical scenarios. In the therapeutic model, the tumor cell line expressing a resistance mutation being targeted by the vaccine is implanted first. Vaccination is administered subsequently. This simulates a scenario of treating patients with pre-existing mutations. In the prophylactic model, vaccination is administered prior to implanting the tumor cell line encoding the resistance mutation included in the vaccine. This scenario mimics treating patients prior to the emergence of acquired mutations. Administration of replicon RNA encoding mutation(s) expressed by the tumor cell line should elicit robust T cell responses in mice that will lead to delayed tumor growth. If tumor growth is not delayed, it is likely that the tumor cell line has evolved to lose the targeted mutation, showing that the replicon RNA was able to exert selective pressure by the immune system to lose the activating mutation.

### Example 5

Construction of Based Alphavirus Vectors Encoding srRNA Constructs for Biotherapeutic Applications

This Example describes the experiments performed to construct base alphavirus vectors (e.g., without a heterologous gene) that were subsequently used for construction of vectors that express a gene or genes of interest (e.g., IL-12 p35 subunit or functional variants thereof, IL-12 p40 subunit or functional variants thereof, and IL-1RA or functional variant thereof).

### EEEV-Base Vector

The base EEEV vector (i.e. without a heterologous gene of interest) was constructed as follows: The base EEEV vector was synthesized de novo in four ˜4 kb parts (Twist Bioscience) from a reference sequence (Genbank EF151502) with several modifications. Silent mutations G301A, A3550C, G4516A, G5725A, G7399A mutations were incorporated to eliminate restriction enzyme cut sites. A unique restriction enzyme cut site (SpeI, 5‘-A’CTAG,T-3′) was incorporated in place of the coding sequence of the native EEEV structural genes (where the 5′ A matches the location of the structural polyprotein ATG start codon, and the 3′ T matches the location of the structural polyprotein stop codon TAA). A 5′ adaptor sequence (5′-CTGGAGACGTGGAGGAGAACCCTGGACCT-3′; SEQ ID NO: 5) was inserted upstream of the SpeI site, and a 3′ adaptor sequence (5′-GACCGCTACGCCCCAATGACCCGACCAGC-3′; SEQ ID NO: 6) was inserted downstream of the SpeI site for subsequent Gibson Assembly® procedures (Gibson et al., Nat. Methods 6, 343-345, 2009). A bacteriophage T7 RNA polymerase promoter (5′-TAATACGACTCACTATAG-3′; SEQ ID NO: 7) was included upstream of the EEEV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 8) followed by a unique restriction enzyme cut site (NotI, 5‘-GC’GGCC,GC-3′). The parts were combined in a five-piece Gibson Assembly® reaction: a linearized pYL backbone and the four synthesized fragments to result in the EEEV base vector.

### CHIKV-Base Vector

The base CHIKV S27 vector was synthesized de novo in four ˜4 kb parts (Twist Bioscience, Thermo Fisher GeneArt) from a reference sequence (Genbank AF369024) with a silent A5366G mutation, and with a unique restriction enzyme cut site (SpeI, 5‘-A’CTAG,T-3′) in place of the coding sequence of the CHIKV structural genes (where the 5′ A matches the location of the structural polyprotein's ATG start codon, and the 3′ T matches the location of the structural polyprotein's stop codon TAA). A 5′ adaptor sequence (5′-CTGGAGACGTGGAGGAGAACCCTGGACCT-3′; SEQ ID NO: 5) was inserted upstream of the SpeI site, and a 3′ adaptor sequence (5′-GACCGCTACGCCCCAATGACCCGACCAGC-3′; SEQ ID NO: 6) was inserted downstream of the SpeI site for subsequent Gibson Assembly® procedures. A bacteriophage T7 RNA polymerase promoter (5′-TAATACGACTCACTATAG-3′; SEQ ID NO: 7) was included upstream of the CHIKV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 8) followed by a unique restriction enzyme cut site (NotI, 5‘-GC’GGCC,GC-3′). The parts were combined in a five-piece Gibson Assembly® reaction a linearized pYL backbone and the four synthesized fragments to result in the CHIKV S27 base vector.

The CHIKV DRDE base vector was similarly constructed from a reference sequence (Genbank EF210157), except the S27 3′ UTR was used in place of the DRDE 3′ UTR.

### SINV-Base Vectors

The base SINV Girdwood vector was synthesized de novo in four ˜4 kb parts (Twist Bioscience, Thermo Fisher GeneArt) from a Girdwood strain reference sequence (Genbank MF459683) with a unique restriction enzyme cut site (SpeI, 5‘-A’CTAG,T-3′) in place of the coding sequence of the SINV structural genes (where the 5′ A is the next nucleotide after a P2A sequence following nucleotide 93 of the structural polyprotein gene, and the 3′ T matches the location of the structural polyprotein's stop codon TGA). A bacteriophage T7 RNA polymerase promoter (5′-TAATACGACTCACTATAG-3′; SEQ ID NO: 7) was included upstream of the SINV genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 8) followed by a unique restriction enzyme cut site (NotI, 5‘-GC’GGCC,GC-3′). The parts were combined in a five-piece Gibson Assembly® reaction (e.g., a linearized pYL backbone and the four synthesized fragments) to result in the SINV Girdwood base vector.

The base SINV AR86 vector was similarly constructed from a reference sequence (Genbank U38305), except the nsP2 coding sequence was derived from the Girdwood reference sequence.

### VEE-Base Vector

The base VEE vector was synthesized de novo in four ˜4 kb parts (Twist Bioscience, Thermo Fisher GeneArt) from a TC-83 strain reference sequence (Genbank L01443) with a silent A2087G mutation, and a unique restriction enzyme cut site (SpeI, 5‘-A’CTAG,T-3′) in place of the coding sequence of the VEE structural genes (where the 5′ A is the next nucleotide after a P2A sequence following nucleotide 93 of the structural polyprotein gene, and the 3′ T matches the location of the structural polyprotein's stop codon TGA). A 5′ adaptor sequence (5′-CTGGAGACGTGGAGGAGAACCCTGGACCT-3′; SEQ ID NO: 5) was inserted upstream of the SpeI site, and a 3′ adaptor sequence (5′-GACCGCTACGCCCCAATGACCCGACCAGC-3′; SEQ ID NO: 6) was inserted downstream of the SpeI site for subsequent Gibson Assembly® procedures. A bacteriophage T7 RNA polymerase promoter (5′-TAATACGACTCACTATAG-3′; SEQ ID NO: 7) was included upstream of the VEE genome sequence, and downstream contained a poly(A) sequence followed by a SapI site, which cuts upstream of the recognition site. Immediately downstream of the SapI site is a T7 terminator sequence (5′-AACCCCTCTCTAAACGGAGGGGTTTTTTT-3′; SEQ ID NO: 8) followed by a unique restriction enzyme cut site (NotI, 5‘-GC’GGCC,GC-3′). The parts were combined in a five-piece Gibson Assembly® reaction (e.g., a linearized pYL backbone and the four synthesized fragments) to result in the VEE base vector.

### Final Vector

Construction of a vectors containing heterologous genes was carried out as follows: the empty base vector was linearized by SpeI digestion. The IL-12A, IL12-B, IL-1RN genes were codon optimized/refactored for human expression in silico and along with the EMCV IRES were synthesized de novo (IDT). The synthetic products were amplified using primers which added either 5′ and 3′ adaptor sequences to the ends of the genes, or primers which added P2A sequences and/or sequences of homology to neighboring gene inserts. The digestion product and PCR products were combined by Gibson Assembly® procedure to result in the final vectors.

### Example 6

In Vitro Evaluation of EEEV Vectors Encoding Biotherapeutic srRNA Constructs

This Example describes the results of in vitro experiments performed to evaluate expression levels of the synthetic srRNA constructs described in Example 5 above, and to investigate any differential behavior thereof (e.g., replication and protein expression).

In vitro transcription: RNA was prepared by in vitro transcription from a SapI-linearized plasmid template with bacteriophage T7 polymerase with either a 5′ ARCA cap (HiScribe™ T7 ARCA mRNA Kit, NEB) or by uncapped transcription (HiScribe™ T7 High Yield RNA Synthesis Kit, NEB) followed by addition of a 5′ cap 1 (Vaccinia Capping System, mRNA Cap 2′-O-Methyltransferase, NEB). RNA was then purified using phenol/chloroform extraction, or column purification (Monarch® RNA Cleanup Kit, NEB). RNA concentration was determined by absorbance at 260 nm (Nanodrop, Thermo Fisher Scientific).

Replication: RNA was transformed by electroporation into BHK-21 or Vero cells (e.g., 4D-Nucleofector™, Lonza). At 15-22 hours following transformation, the cells were fixed and permeabilized (eBioscience™ Foxp3/Transcription Factor Staining Buffer Set, Invitrogen) and stained using a PE-conjugated anti-dsRNA mouse monoclonal antibody (J2, Scicons) to quantify the frequency of dsRNA+ cells and the mean fluorescence intensity (MFI) of dsRNA in individual cells by fluorescence flow cytometry.

### Protein Expression by ELISA:

Human IL-12p70 and IL-1RA were detected from electroporated BHK-21 cells with 500 ng of srRNA monogenic or multigenic constructs. Supernatants were harvested at approximately 24 and 48 hours after transfection and assayed with Human IL-12p70 from RnD Systems (cat #DY1270) and Human IL-1ra/IL-1F3 DuoSet ELISA from RnD Systems (cat #DY280).

### Bioactivity Assay:

Human IL-12p70 and IL-1RA were detected from electroporated BHK-21 cells with 500 ng of srRNA monogenic or multigenic constructs. Supernatants were harvested at approximately 24 and 48 hours after transfection and assayed with GloMax bioassay from Promega for IL-12 (cat #JA2601). IL-1RA was assayed using IL-1β Reporter HEK 293 Cells (Invivogen, hkb-il1bv2) in the pre-incubated with 4 ng/mL IL1β (1 ng/mL final) and supernatants from transfected BHK cells as per the manufacturer's protocol.

### Evaluation of Number and Ordinality of Genes

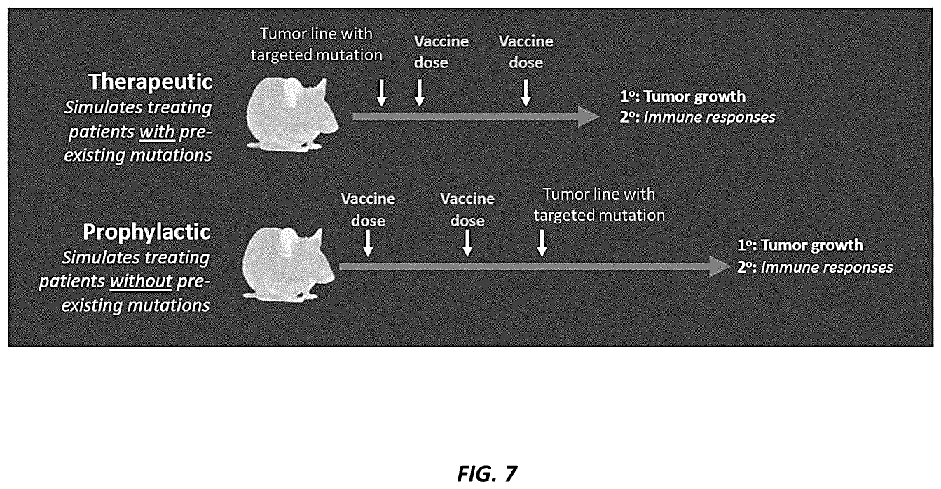

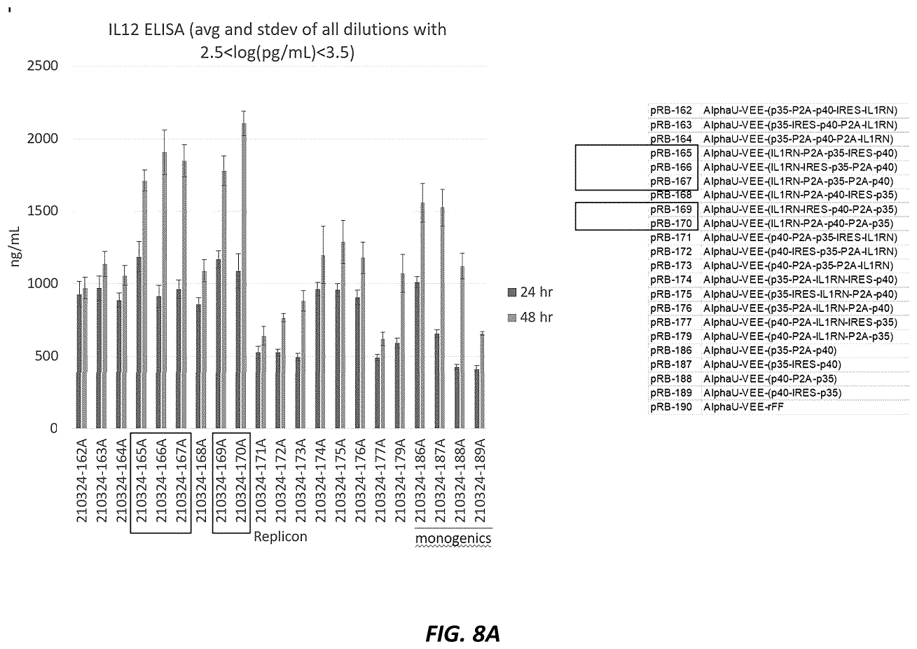

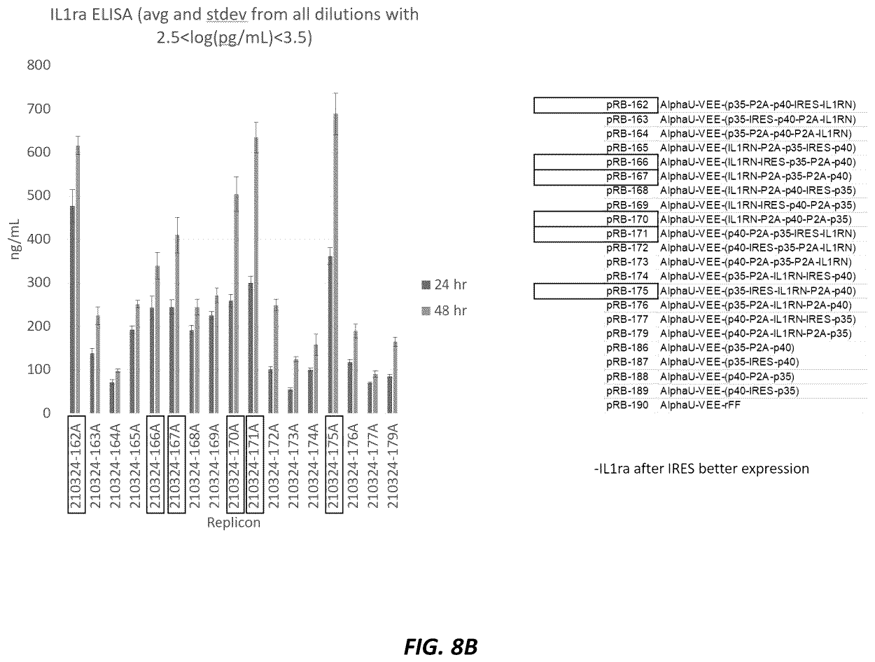

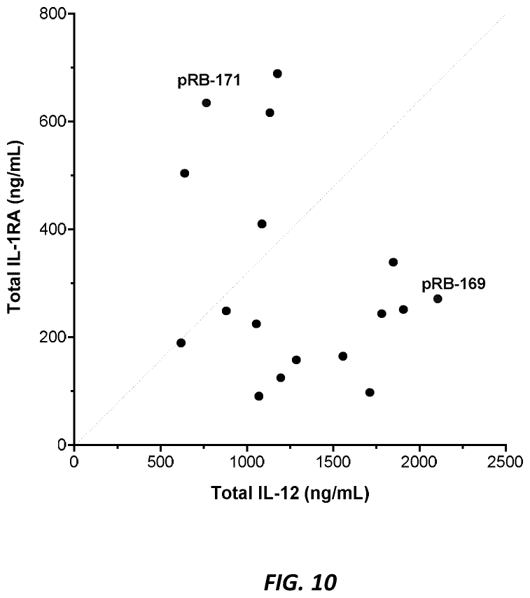

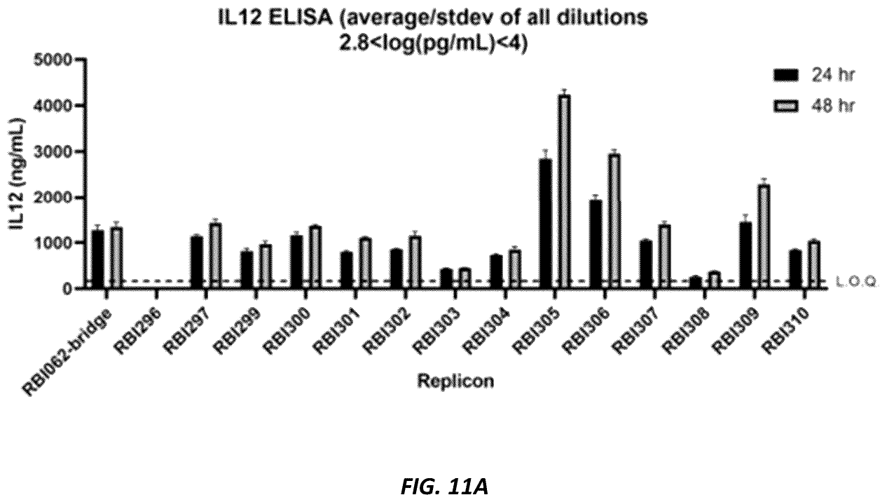

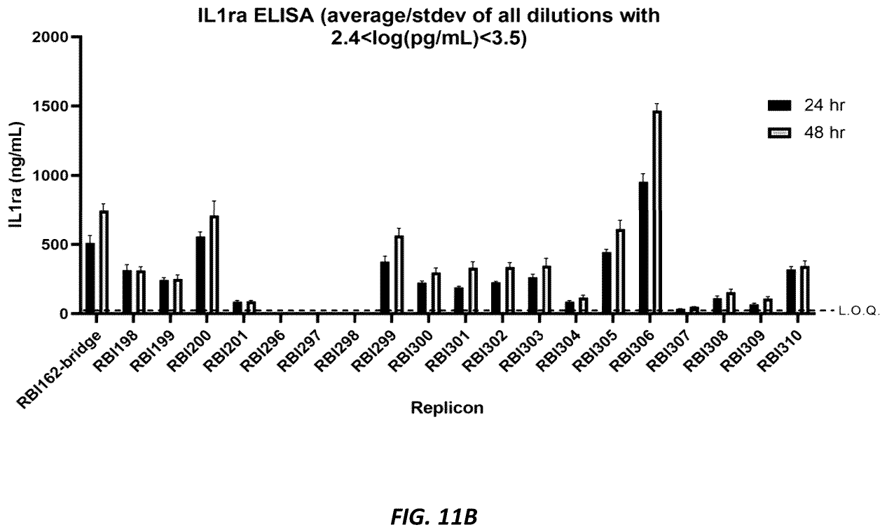

Results of mouse IL-12 and IL-1RA detecting ELISA assays as measured from transfected BHK-21 cells is shown in FIGS. 8A, 8B, 11A, and 11B. Different constructs, in either monogenic or multigenic form, having different ordinalities of IL-12 subunit p35, IL-12 subunit p40, and IL-1RA, were tested in order to determine which configuration of genes in the constructs yielded the most robust expression of IL-12 and IL-1RA. The Y-axis shows IL-12 or IL-1RA concentration in ng/mL. FIG. 10 shows the corresponding IL-12 and IL-1RA concentrations measured from each construct tested.

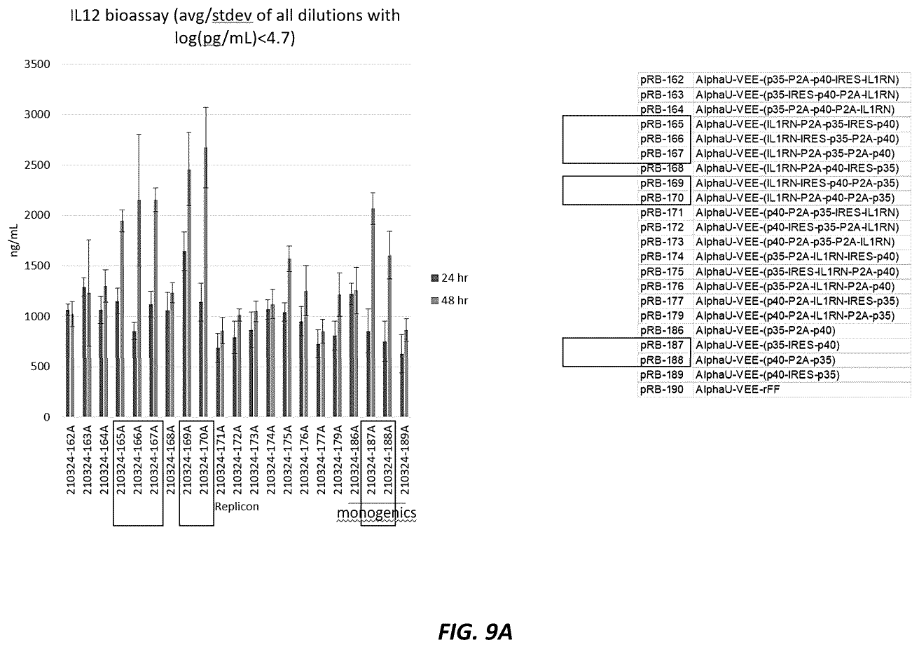

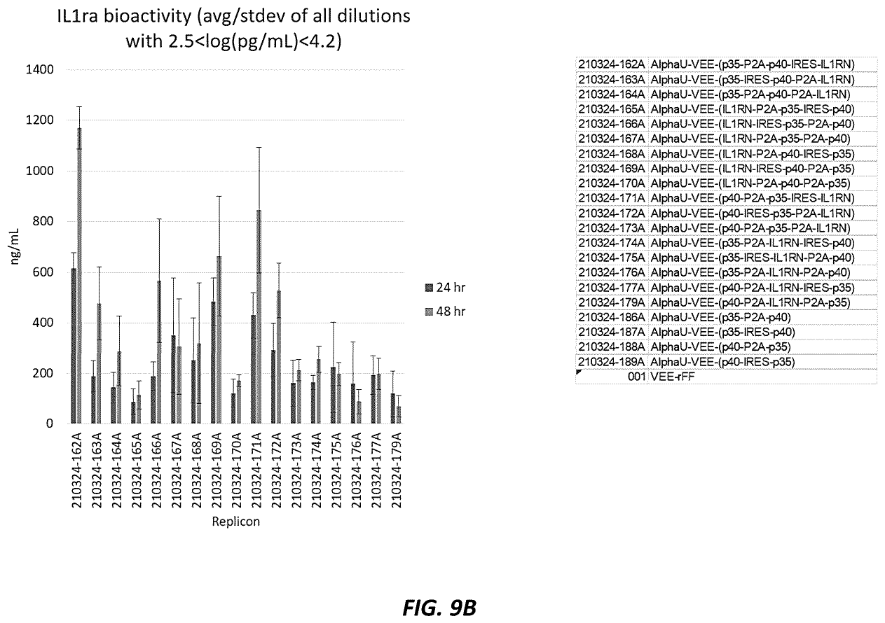

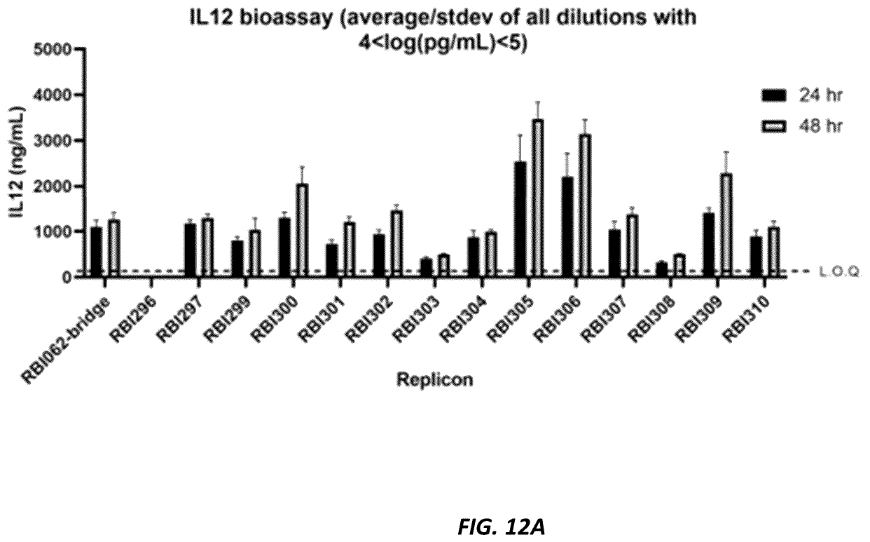

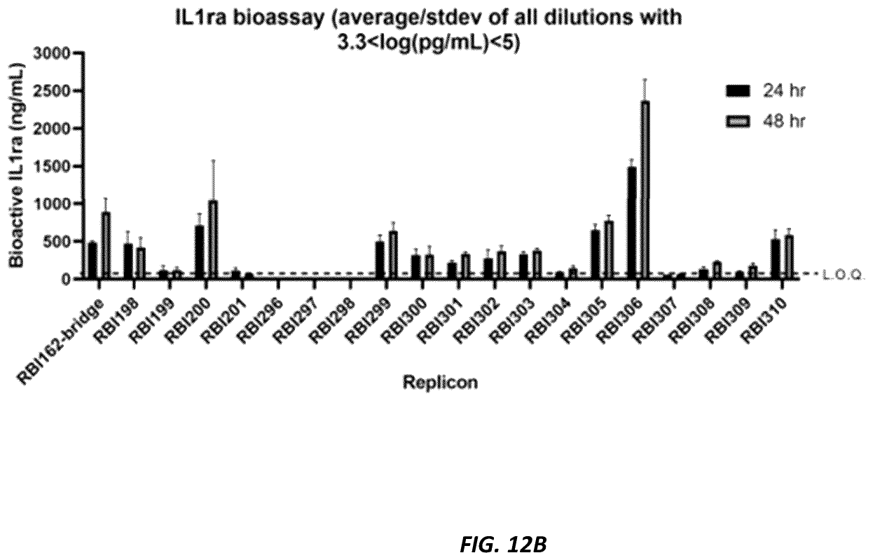

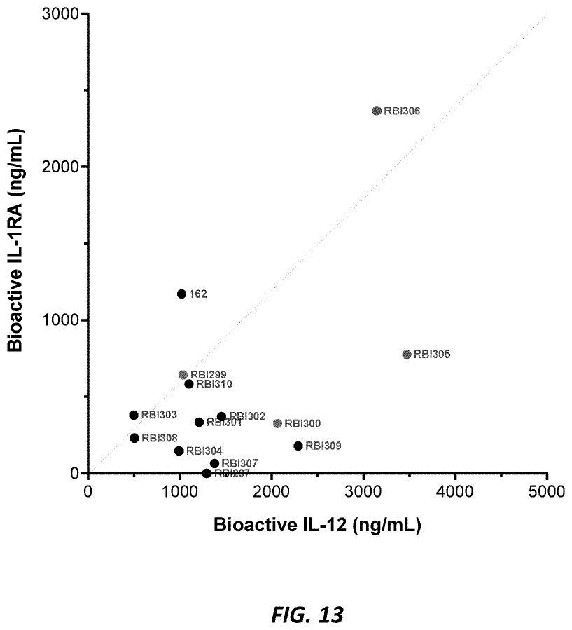

Mouse IL-12 and IL-1RA detecting bioactivity was measured from supernatants of transfected BHK-21 cells with different srRNA constructs tested on reporter cells expressing different cytokine receptors. Results are shown in FIGS. 9A, 9B, 12A, and 12B. FIG. 13 shows the corresponding IL-12 and IL-1RA bioactivities measured from each construct tested.

### Example 7

In Vivo Evaluation of Alphavirus Vectors Encoding Biotherapeutic srRNA Constructs

This Example describes the results of in vivo experiments performed to evaluate the srRNA constructs described herein (e.g., both unformulated and LNP formulated vectors).

In these experiments, synthetic srRNA constructs derived from various alphavirus strains were designed and subsequently evaluated.

Mice and injections. BALB/c mice were purchased from Charles River Labs, Envigo, or Jackson Laboratories. On day of dosing, between 0.01-40 μg of material was injected intramuscularly either into one or split into both quadricep muscles. Vectors were administered either unformulated in saline, or LNP-formulated. Animals were monitored for body weight and other general observations throughout the course of the study. For pharmacokinetic studies, animals were dosed on Day 0 only.

LNP formulation. Replicon RNA was formulated in lipid nanoparticles using a microfluidics mixer and analyzed for particle size, polydispersity using dynamic light scattering and encapsulation efficiency. Lipids were suspended in ethanol. For L1, RNA was suspended in 10 mM citrate buffer pH 5.0 a concentration of 172 μg/ml, and was mixed at a flow rate of 3:1 (aqueous:organic). For L2, RNA was suspended in 250 mM NaOAc pH 4.0 at a concentration of 82 ug/ml, and was mixed at a flow rate of 3:1 (aqueous:organic).

ELISA. To measure the serum concentrations of IL-12 and IL-1RA, ELISA analysis was performed using Human IL-12 p70 DuoSet ELISA (RnD Systems cat #DY1270) and Human IL-1ra ELISA Kit (Abcam, ab211650) as per manufacturer's protocol.

### Evaluation of Ordinality of Genes and Lipid Formulation

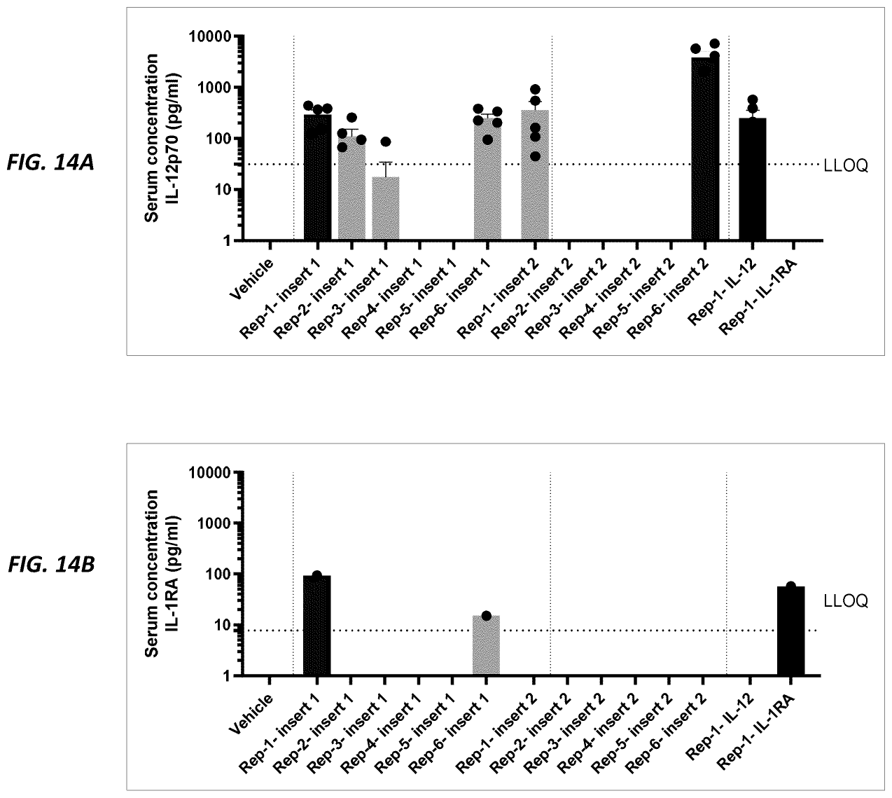

The two best multigenic configurations from in vitro assays were then tested in six different srRNA vectors in vivo for protein expression in mouse serum. Results are shown in FIGS. 14A and 14B. It was observed that IL-1RA was not detected in some instances potentially due to short protein half-life.

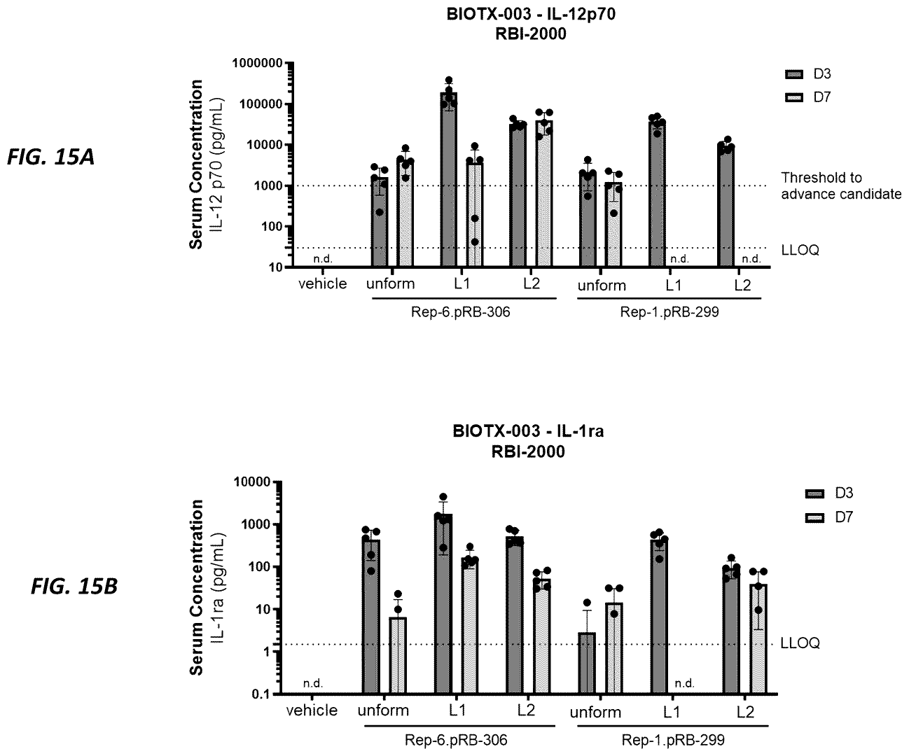

Multigenic configurations were then tested in different formulations and analyzed for protein expression in vivo by ELISA. Results are shown in FIGS. 15A and 15B.

### Example 8

In Vivo Evaluation of Modified srRNA Vectors with Heterologous Nonstructural Protein Genes

This Example describes the results of in vivo experiments performed to evaluate the differential functionality of a polypeptide construct of interest (PCI) expressed from a plurality of LNP formulated srRNA expression constructs to elicit immune responses and establish immune protection following vaccination.

In these experiments, synthetic srRNA constructs derived from Venezuelan equine encephalitis virus (VEE.TC83), Chikungunya virus strains S27 (CHIK.S27) and DRDE-06 (CHIK.DRDE), Sindbis virus strains Girdwood (SIN.GW) and AR86-Girdwood Hybrid 1 (SIN.AR86), and Eastern equine encephalitis virus (EEE.FL93) were designed and subsequently evaluated.

Mice and injections. Female BALB/c mice were purchased from Charles River Labs or Jackson Laboratories. On day of dosing, between 0.15-1.5 μg of material was injected intramuscularly split into both quadricep muscles. Vectors were LNP-formulated. Animals were monitored for body weight and other general observations throughout the course of the study. For immunogenicity studies, animals were dosed on Day 0 and Day 21. Spleens were collected at Day 14 and/or 35, and serum was isolated at Days 14, and/or 35.

LNP formulation. srRNA was formulated in lipid nanoparticles (LNPs) using a microfluidics mixer and analyzed for particle size, polydispersity using dynamic light scattering and encapsulation efficiency. LNPs are composed of an ionizable lipid, cholesterol, PEG-2K, and DOPE.

ELISpot. To measure the magnitude of antigen-specific T cell responses, IFNγ ELISpot analysis was performed using Mouse IFNγ ELISpot PLUS Kit (HRP) (MabTech) as per manufacturer's instructions. In brief, splenocytes are isolated and resuspended to a concentration of 1-5×10⁶ cells/mL in media containing either peptide pools corresponding to rabies virus glycoprotein G, PMA/ionomycin as a positive control, or DMSO as a mock stimulation.

Antibodies. Neutralizing antibody responses to rabies virus were measured using the Rapid Fluorescent Focus Inhibition Test. In brief, serum dilutions were mixed with a standard amount of live rabies virus and incubated. If neutralizing anti-rabies antibodies are present, they will neutralize the virus. Next, cultured cells were added and the serum/virus/cells were incubated together. Uncoated rabies virus (i.e. that has not been neutralized by antibodies), will infect the cells and this can be visualized by microscopy. Calculation of the endpoint titer was made from the percent of virus infected cells observed on the slide.

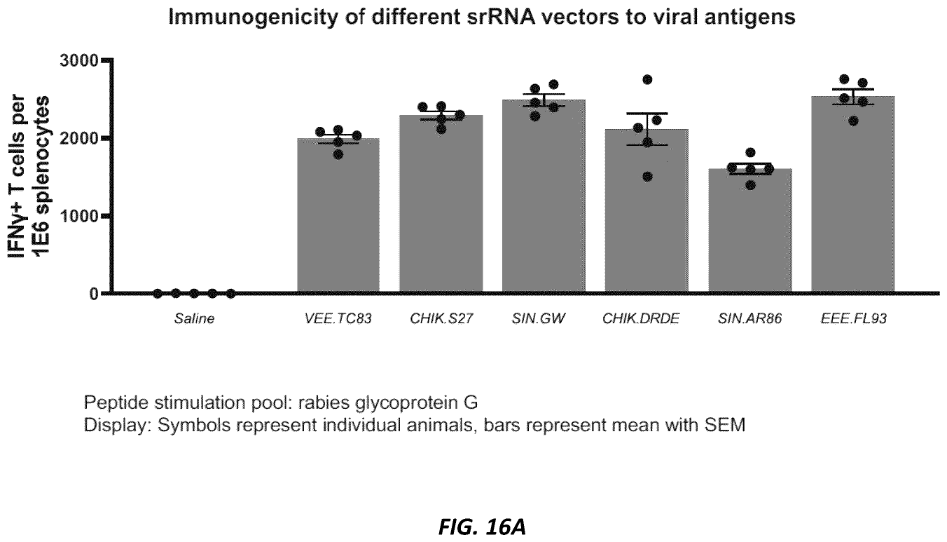

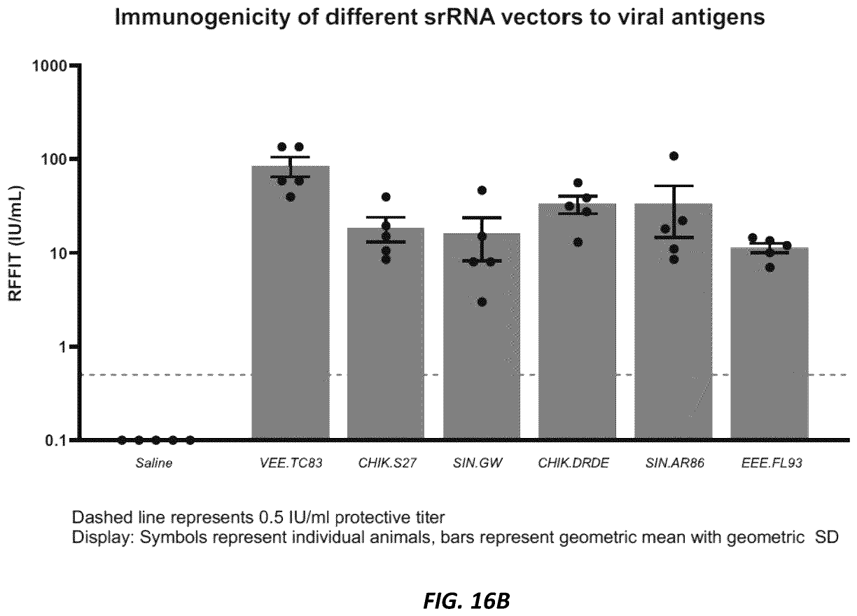

In vivo immunogenicity of a plurality of srRNAs encoding a viral antigen, rabies virus glycoprotein G, was assessed by evaluating antigen-specific splenic T cell responses by ELISpot (FIG. 16A) and anti-rabies neutralizing antibody titers from sera (FIG. 16B) after two immunizations. All srRNA-immunized groups showed robust T cell responses compared to saline controls (FIG. 16A), but differential responses were observed between srRNA vaccines. Similarly, all srRNA-immunized groups showed protective neutralizing antibody titers with some variations between srRNA vaccines (FIG. 16B).

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

What is claimed is:

1. A method for identifying and/or characterizing a self-replicating RNA (srRNA) construct, the method comprising:
a) providing a plurality of srRNA expression constructs each comprising a coding sequence for a polypeptide construct of interest (PCI) operably inserted into an alphavirus srRNA vector, wherein at least a portion of the coding sequence for the alphavirus structural proteins has been replaced with the coding sequence of the PCI;
b) analyzing level and/or functionality of the PCIs that are expressed from the plurality of srRNA expression constructs to identify one or more candidate PCIs having a defined property;
c) incorporating the srRNA expression constructs capable of expressing the candidate PCIs identified in (b) with at least one delivery vehicle to create a combinatorial collection of delivery systems; and
d) analyzing the delivery systems for their capability to confer at least one pharmacodynamic effect in a subject to identify a srRNA expression construct capable of conferring a desired pharmacodynamic effect.

2. The method of claim 1, wherein the coding sequence for the PCI comprises a coding sequence for a single polypeptide or coding sequences for a plurality of polypeptides.

3. The method of claim 2, wherein the coding sequences of the plurality of polypeptides are operably linked to one another within a single open reading frame (i.e., in a polycistronic ORF).

4. The method of claim 2, wherein the plurality of polypeptides are operably linked to one another by one or more connector sequences.

5. The method of claim 4, wherein a connector sequence of the plurality of connector sequences comprises an autoproteolytic peptide sequence.

6. The method of claim 5, wherein the autoproteolytic peptide sequence comprises one or more autoproteolytic cleavage sequences derived from a calcium-dependent serine endoprotease (furin), a porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2A (BmCPV2A), a Flacherie Virus 2A (BmIFV2A), or a combination thereof.

7. The method of any one of claims 4 to 6, wherein the coding sequences of the plurality of polypeptides are operably linked to one another by one or more an internal ribosomal entry sites (IRES).

8. The method of claim 7, wherein the one or more IRES is selected from a viral IRES, a cellular IRES, and an artificial IRES.

9. The method of any one of claims 7 to 8, wherein the one or more IRES is selected from a Kaposi's sarcoma-associated herpesvirus (KSHV) IRES, a hepatitis virus IRES, a Pestivirus IRES, a Cripavirus IRES, a Rhopalosiphum padi virus IRES, a fibroblast growth factor IRES, a platelet-derived growth factor IRES, a vascular endothelial growth factor IRES, an insulin-like growth factor IRES, a picornavirus IRES, an encephalomyocarditis virus (EMCV) IRES, a Pim-1 IRES, a p53 IRES, an Apaf-1 IRES, a TDP2 IRES, an L-myc IRES, and a c-myc IRES.

10. The method of any one of claims 1 to 9, wherein the PCI comprises one or more polypeptides selected from microbial proteins, viral proteins, bacterial proteins, fungal proteins, mammalian proteins, and combinations of any thereof.

11. The method of any one of claims 1 to 10, wherein the PCI comprises one or more polypeptides selected from antigen molecules, biotherapeutic molecules, or combinations of any thereof.

12. The method of any one of claims 1 to 11, wherein the PCI comprises one or more antigen polypeptides selected from tumor-associated antigens, tumor-specific antigens, neoantigens, and combinations of any thereof.

13. The method of claim 12, wherein the one or more antigen polypeptides comprises estrogen receptors, intracellular signal transducer enzymes, and human epidermal growth receptors.

14. The method of any one of claims 10 to 13, wherein the one or more antigen polypeptides is selected from ESR1, PI3K, HER2, HER3, variants of any thereof, and combinations of any thereof.

15. The method of any one of claims 1 to 11, wherein the PCI comprises one or more biotherapeutic polypeptides selected from immunomodulators, modulators of angiogenesis, modulators of extracellular matrix, modulators of metabolism, neurological modulators, and combinations of any thereof.

16. The method of claim 15, wherein the PCI comprises one or more cytokines selected from chemokines, interferons, interleukins, lymphokines, and tumor necrosis factors.

17. The method of claim 16, wherein the PCI comprises one or more interleukins selected from IL-1α, IL-1β, IL-2, IL-3, IL-4, IL-5, IL-6, IL-7, IL-8, IL-9, IL-10, IL-11, IL-12, IL-15, IL-15, IL-17, IL-23, IL-27, IL-35, IFNγ, and subunits of any thereof.

18. The method of any one of claims 10 to 17, wherein the one or more biotherapeutic polypeptides is selected from IL-12A, IL-12B, IL-1RA, and combinations of any thereof.

19. The method of any one of claims 1 to 18, wherein step (a) comprises providing a plurality of srRNA expression constructs each comprising a coding sequence for a variant of the PCI.

20. The method of claim 19, wherein the providing in (a) comprises:
i) obtaining an alphavirus srRNA expression vector, wherein at least a portion of encoding sequence for the alphavirus structural proteins has been replaced with a coding sequence for a polypeptide construct of interest (PCI); and
ii) generating a plurality of srRNA expression constructs each comprising a coding sequence for a variant of the PCI.

21. The method of claim 19, wherein the providing in (a) comprises:
i) obtaining coding sequences for a plurality of variants of a polypeptide construct of interest (PCI); and
ii) generating a plurality of srRNA expression constructs each comprising a coding sequence for a PCI variant of the plurality of PCI variants from (a) operably inserted into an alphavirus srRNA vector, wherein at least a portion of the encoding sequence for the alphavirus structural proteins has been replaced with the coding sequence of the PCI variant.

22. The method of any one of claims 20 to 21, wherein a PCI variant of the plurality of PCI variants comprises one or more molecular alterations.

23. The method of claim 22, wherein the one or more molecular alterations in the PCI variant is selected from the group consisting of deletions, substitutions, insertion, duplications, mutations, frameshift variants, splice variants, and combinations of any thereof.

24. The method of any one of claims 22 to 23, wherein the one or more molecular alterations are configured into a plurality of alteration cassettes arranged in tandem along the length of the antigen sequence.

25. The method of claim 24, wherein an alteration cassette of the plurality of alteration cassettes comprises one, two, three, four, five, or more molecular alterations.

26. The method of any one of claims 24 to 25, wherein the plurality of alteration cassettes are operably linked to one another by one or more linkers.

27. The method of claim 26, wherein a linker of the one or more linkers comprises a synthetic compound linker or a peptide linker.

28. The method of claim 27, wherein the peptide linker comprises an amino acid sequence selected from the group consisting of AAY, EAAAK (SEQ ID NO: 1), RVRR (SEQ ID NO: 2), GGGGS (SEQ ID NO: 3), and GPGPG (SEQ ID NO: 4).

29. The method of any one of claims 1 to 28, wherein the analyzing level and/or functionality of the PCIs in step (b) is carried out in vitro, in vivo, or ex vivo.

30. The method of claim 29, wherein the analyzing level and/or functionality of the PCIs comprises immunoblotting analysis, fluorescence flow cytometry analysis, enzyme-linked immunoassay analysis, immunogenicity analysis, bioactivity analysis, and/or efficacy in a disease model.

31. The method of any one of claims 1 to 29, wherein the analysis of the delivery systems for theirs capacity to confer at least one pharmacodynamic effects in step (d) is carried out in vivo or ex vivo.

32. The method of any one of claims 1 to 31, wherein the at least one pharmacodynamic effects comprises one or more of the following: immunogenicity effect, a biomarker response, a therapeutic effect, a prophylactic effect, a desired effect, an undesired effect, an adverse effect, and effect in a disease model.

33. The method of claim 32, wherein the at least one pharmacodynamic effects comprises induction of an immune response.

34. The method of any one of claims 1 to 33, wherein the delivery systems comprise a physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof.

35. The method of claim 34, wherein the LNP delivery system comprises a cationic lipid, an ionizable cationic lipid, an anionic lipid, or a neutral lipid.

36. The method of claim 34, wherein the LNP delivery system comprises an ionizable cationic lipid selected from the group consisting of ALC-0315, C12-200, LN16, MC3, MD1, SM-102, and a combination of any thereof.

37. The method of claim 34, wherein the LNP comprises a cationic lipid selected from the group consisting of 98N12-5, C12-200, C14-PEG2000, DLin-KC2-DMA (KC2), DLin-MC3-DMA (MC3), XTC, MD1, 7C1, and a combination of any thereof.

38. The method of claim 34, wherein the LNP comprises a neutral lipid selected from the group consisting of DPSC, DPPC, POPC, DOPE, SM, and a combination of any thereof.

39. The method of claim 34, wherein the LNP comprises lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, and GL67A-DOPE-DMPE-polyethylene glycol (PEG).

40. The method of any one of claims 34 to 39, wherein the mass ratio of lipid to nucleic acid in the LNP delivery system is about 100:1 to about 3:1, about 70:1 to 10:1, or 16:1 to 4:1.

41. The method of claim 40, wherein the mass ratio of lipid to nucleic acid in the LNP delivery system is about 16:1 to 4:1.

42. The method of any one of claims 40 to 41, wherein the mass ratio of lipid to nucleic acid in the LNP delivery system is about 20:1.

43. The method of any one of claim 40 to 41, wherein the mass ratio of lipid to nucleic acid in the LNP delivery system is about 8:1.

44. The method of any one of claims 34 to 43, wherein the lipid-based nanoparticles (LNPs) have an average diameter of less than about 1000 nm, about 500 nm, about 250 nm, about 200 nm, about 150 nm, about 100 nm, about 75 nm, about 50 nm, or about 25 nm.

45. The method of claim 44, wherein the LNPs have an average diameter ranging from about 70 nm to 100 nm.

46. The method of claim 45, wherein the LNPs have an average diameter ranging from about 88 nm to about 92 nm, from 82 nm to about 86 nm, or from about 80 nm to about 95 nm.

47. The method of any one of claims 1 to 46, wherein the recombinant alphavirus srRNA is of a virus belonging to the Alphavirus genus of the Togaviridae family.

48. The method of claim 47, wherein the recombinant alphavirus srRNA is of an alphavirus belonging to the VEEV/EEEV group, or the SFV group, or the SINV group.

49. The method of claim 48, wherein the alphavirus is Eastern equine encephalitis virus (EEEV), Venezuelan equine encephalitis virus (VEEV), Everglades virus (EVEV), Mucambo virus (MUCV), Pixuna virus (PIXV), Middleburg virus (MIDV), Chikungunya virus (CHIKV), O'Nyong-Nyong virus (ONNV), Ross River virus (RRV), Barmah Forest virus (BF), Getah virus (GET), Sagiyama virus (SAGV), Bebaru virus (BEBV), Mayaro virus (MAYV), Una virus (UNAV), Sindbis virus (SINV), Aura virus (AURAV), Whataroa virus (WHAV), Babanki virus (BABV), Kyzylagach virus (KYZV), Western equine encephalitis virus (WEEV), Highland J virus (HJV), Fort Morgan virus (FMV), Ndumu virus (NDUV), Madariaga virus (MADV), or Buggy Creek virus.

50. The method of claim 49, wherein the alphavirus is Venezuelan equine encephalitis virus (VEEV), Eastern Equine Encephalitis virus (EEEV), Chikungunya virus (CHIKV), or Sindbis virus (SINV).

51. The method of any one of claims 1 to 50, wherein the srRNA expression vector is identified as having an immune-inducing activity suitable for a prophylactic use and/or a therapeutic use.

52. A self-replicating RNA (srRNA) construct identified according to a method of claims 1 to 51.

53. A nucleic acid encoding a srRNA construct according to claim 52.

54. A recombinant cell comprising:
a) a self-replicating RNA construct according to claim 52; and/or
b) a nucleic acid according to claim 53.

55. The recombinant cell of claim 54, wherein the recombinant cell is a eukaryotic cell.

56. The recombinant cell of any one of claims 54 to 55, wherein the recombinant cell is an animal cell.

57. The recombinant cell of claim 56, wherein the animal cell is a vertebrate animal cell or an invertebrate animal cell.

58. The recombinant cell of claim 56, wherein the animal cell is a mammalian cell.

59. The recombinant cell of claim 56, wherein the animal cell is an insect cell.

60. The recombinant cell of claim 59, wherein the insect cell is a mosquito cell.

61. The recombinant cell of claim 56, wherein the animal cell is an immune cell.

62. The recombinant cell of claim 61, wherein the immune cell is a B cell, a monocyte, a natural killer (NK) cell, a natural killer T (NKT) cell, a basophil, an eosinophil, a neutrophil, a dendritic cell (DC), a macrophage, a regulatory T cell, a helper T cell (T_{H}), a cytotoxic T cell (T_{CTL}), a memory T cell, a gamma delta (γδ) T cell, a hematopoietic stem cell, or a hematopoietic stem cell progenitor.

63. The recombinant cell of claim 62, wherein the immune cell is a B cell, a T cell, or a dendritic cell (DC).

64. A cell culture comprising at least one recombinant cell according to any one of claims 54 to 63, and a cell culture medium.

65. A composition comprising a therapeutically acceptable excipient and:
a) a self-replicating RNA construct according to claim 52;
b) a nucleic acid according to claim 53; and/or
b) a recombinant cell according to any one of claims 54 to 63.

66. The composition of claim 65, wherein the composition is formulated as a vaccine.

67. The composition of claim 66, wherein the vaccine is a therapeutic vaccine.

68. The composition of any one of claims 65 to 67, wherein the composition is formulated as an immunogenic composition.

69. The composition of any one of claims 65 to 66, wherein the composition is formulated as a biotherapeutic.

70. The composition of any one of claims 65 to 69, wherein the composition is formulated with a delivery vehicle into a delivery system.

71. The composition of claim 70, wherein the delivery system comprises a physiologic buffer, a liposome, a lipid-based nanoparticle (LNP), a polymer nanoparticle, a viral replicon particle (VRP), a microsphere, an immune stimulating complex (ISCOM), a conjugate of bioactive ligand, or a combination of any thereof.

72. The composition of claim 71, wherein the LNP delivery system comprises a cationic lipid, an ionizable cationic lipid, an anionic lipid, or a neutral lipid.

73. The composition of claim 71, wherein the LNP comprises an ionizable cationic lipid selected from the group consisting of ALC-0315, C12-200, LN16, MC3, MD1, SM-102, and a combination of any thereof.

74. The composition of claim 71, wherein the LNP comprises a cationic lipid selected from the group consisting of 98N12-5, C12-200, C14-PEG2000, DLin-KC2-DMA (KC2), DLin-MC3-DMA (MC3), XTC, MD1, 7C1, and a combination of any thereof.

75. The composition of claim 71, wherein the LNP comprises a neutral lipid selected from the group consisting of DPSC, DPPC, POPC, DOPE, SM, and a combination of any thereof.

76. The composition of claim 71, wherein the LNP comprises a lipid selected from the group consisting of C12-200, C14-PEG2000, DOPE, DMG-PEG2000, DSPC, DOTMA, DOSPA, DOTAP, DMRIE, DC-cholesterol, DOTAP-cholesterol, GAP-DMORIE-DPyPE, GL67A-DOPE-DMPE-polyethylene glycol (PEG), and a combination of any thereof.

77. The composition of any one of claims 71 to 76, wherein the mass ratio of lipid to nucleic acid in the LNP delivery system is about 100:1 to about 3:1, about 70:1 to 10:1, or 16:1 to 4:1.

78. The composition of claim 77, wherein the mass ratio of lipid to nucleic acid in the LNP delivery system is about 16:1 to 4:1.

79. The composition of claim 78, wherein the mass ratio of lipid to nucleic acid in the LNP delivery system is about 20:1.

80. The composition of claim 78, wherein the mass ratio of lipid to nucleic acid in the LNP delivery system is about 8:1.

81. The composition of any one of claims 71 to 80, wherein the lipid-based nanoparticles (LNPs) have an average diameter of less than about 1000 nm, about 500 nm, about 250 nm, about 200 nm, about 150 nm, about 100 nm, about 75 nm, about 50 nm, or about 25 nm.

82. The composition of claim 81, wherein the LNPs have an average diameter ranging from about 70 nm to 100 nm.

83. The composition of claim 82, wherein the LNPs have an average diameter ranging from about 88 nm to about 92 nm, from 82 nm to about 86 nm, or from about 80 nm to about 95 nm.

84. A method for inducing a pharmacodynamic effect in a subject, the method comprising administering to the subject a composition comprising;
a) a self-replicating RNA construct according to claim 52;
b) a nucleic acid according to claim 53;
c) a recombinant cell according to any one of claims 54 to 63; and/or
d) a pharmaceutical composition according to any one of claims 65 to 83.

85. The method of claim 84, wherein the pharmacodynamic effect comprises eliciting an immune response in the subject.

86. A method for preventing or treating a health condition in a subject, the method comprising prophylactically or therapeutically administering to the subject a composition comprising;
a) a self-replicating RNA construct according to claim 52;
b) a nucleic acid according to claim 53;
c) a recombinant cell according to any one of claims 54 to 63; and/or
d) a pharmaceutical composition according to any one of claims 65 to 83.

87. The method of claim 86, wherein the administered composition elicits an immune response in the subject.

88. The method of any one of claims 84 to 87, wherein the administered composition induces production of one or more pro-inflammatory molecules in the subject.

89. The method of claim 88, wherein the one or more pro-inflammatory molecules comprises interferon gamma (IFNγ), cytokines, TNF-α, GM-CSF, and MIP1α, granzyme B, granzyme A, perforin, or a combination of any thereof.

90. The method of any one of claims 84 to 89, wherein the subject has been previously treated with one or more therapies and has developed at least a partial resistance to said one or more therapies.

91. The method of claim 90, wherein at least one of the one or more therapies comprises a small molecule.

92. The method of any one of claims 84 to 91, wherein the health condition is a proliferative disorder, inflammatory disorder, autoimmune disorder, or a microbial infection.

93. The method of any one of claims 84 to 92, wherein the subject has or is suspected of having a health condition associated with proliferative disorder, inflammatory disorder, autoimmune disorder, or a microbial infection.

94. The method of any one of claims 92 to 93, wherein the proliferative disorder is a cancer.

95. The method of claim 94, wherein the cancer is a breast cancer.

96. The method of any one of claims 84 to 95, wherein the composition is administered to the subject individually as a single therapy (monotherapy) or as a first therapy in combination with at least one additional therapies.

97. The method of claim 96, wherein the at least one additional therapies is selected from the group consisting of chemotherapy, radiotherapy, immunotherapy, hormonal therapy, toxin therapy, targeted therapy, and surgery.

98. A kit for inducing a pharmacodynamic effect, eliciting an immune response, and/or for the prevention and/or treatment of a health condition, the kit comprising one or more of the following:
a) a self-amplifying RNA construct according to claim 52;
b) a nucleic acid according to claim 53;
c) a recombinant cell according to any one of claims 54 to 63; and
d) a pharmaceutical composition according to any one of claims 65 to 83.


SEQUENCE LISTING (8 sequences)

SEQ ID NO: 1 - protein, 5 aa, synthetic construct
  REGION 1..5: note = Synthetic construct
  REGION 1..5: note = MISC_FEATURE - peptide linker
EAAAK

SEQ ID NO: 2 - protein, 4 aa, synthetic construct
  REGION 1..4: note = Synthetic construct
  REGION 1..4: note = MISC_FEATURE - peptide linker
RVRR

SEQ ID NO: 3 - protein, 5 aa, synthetic construct
  REGION 1..5: note = Synthetic construct
  REGION 1..5: note = MISC_FEATURE - peptide linker
GGGGS

SEQ ID NO: 4 - protein, 5 aa, synthetic construct
  REGION 1..5: note = Synthetic construct
  REGION 1..5: note = MISC_FEATURE - peptide linker
GPGPG

SEQ ID NO: 5 - DNA, 29 bp, synthetic construct
  misc_feature 1..29: note = Synthetic construct
  misc_feature 1..29: note = MISC_FEATURE - 5' adaptor sequence
  source 1..29: mol_type = other DNA
ctggagacgtggaggagaaccctggacct

SEQ ID NO: 6 - DNA, 29 bp, synthetic construct
  misc_feature 1..29: note = Synthetic construct
  misc_feature 1..29: note = MISC_FEATURE - 3' adaptor sequence
  source 1..29: mol_type = other DNA
gaccgctacgccccaatgacccgaccagc

SEQ ID NO: 7 - DNA, 18 bp, synthetic construct
  misc_feature 1..18: note = Synthetic construct
  misc_feature 1..18: note = MISC_FEATURE - bacteriophage T7 RNA polymerase promoter
  source 1..18: mol_type = other DNA
taatacgactcactatag

SEQ ID NO: 8 - DNA, 29 bp, synthetic construct
  misc_feature 1..29: note = Synthetic construct
  misc_feature 1..29: note = MISC_FEATURE - T7 terminator sequence
  source 1..29: mol_type = other DNA
aacccctctctaaacggaggggttttttt